(19) United States
(12) Patent Application Publication
(10) Pub. No.: US 2007/0015813 A1
(43) Pub. Date: Jan. 18, 2007

(54) Treatment of protein folding disorders

(75) Inventors: Michael D. Carter, Nova Scotia (CA); Mark Hadden, Albany, NY (US); Donald F. Weaver, Nova Scotia (CA); Sheila Marie H. Jacobo, Nova Scotia (CA); Erhu Lu, Nova Scotia (CA)

Correspondence Address: DAVIDSON, DAVIDSON & KAPPEL, LLC, 485 SEVENTH AVENUE, 14TH FLOOR, NEW YORK, NY, 10018, US

(73) Assignee: QUEEN'S UNIVERSITY AT KINGSTON, Ontario (CA)

(21) Appl. No.: 11/443,396

(22) Filed: May 30, 2006

Related U.S. Application Data
(60) Provisional application No. 60/685,609, filed on May 27, 2005.
(60) Provisional application No. 60/685,610, filed on May 27, 2005.
(60) Provisional application No. 60/685,369, filed on May 27, 2005.
(60) Provisional application No. 60/709,474, filed on Aug. 19, 2005.
(60) Provisional application No. 60/719,615, filed on Sep. 22, 2005.
(60) Provisional application No. 60/788,519, filed on Mar. 31, 2006.

(51) Int. Cl. A61K 31/405

(52) U.S. Cl. 514/414

(57) ABSTRACT

In certain embodiments, the invention is directed to a method for treating a protein folding disorder comprising administering to a subject a compound of the formulas disclosed. In preferred embodiments, the compounds are bis-indole compounds.

### BACKGROUND OF THE INVENTION

Protein folding disorders include neurodegenerative conditions such as, e.g, Alzheimer's disease, dementia, Huntington's disease, Parkinson's disease and prion-based spongiform encephalopathy (e.g., Creutzfeldt-Jakob disease).

Alzheimer's disease (AD) is a progressive neurodegenerative disease which first manifests with mild cognitive, memory and behavioral symptoms that gradually worsen in severity and eventually lead to dementia. It is the most common cause of dementia, accounting for between 42 and 81% of cases, as determined in various studies (Nussbaum, R L; Ellis, C E. N Engl J Med, 2003, 348:1356-64). It affects 2.5% of people 65-74 years of age, 4% of people aged 75-79, 11% of those aged 80-84, and 24% of those aged 85-93 years (Siegel, G J; Agranoff, B W; Albers, R W; Molinoff, P B, Basic Neurochemistry. Fifth ed. 1994, New York: Raven Press, 1054 pp). Accounting for 100,000 deaths annually in North America alone, AD is the fourth leading cause of death in industrialized societies, preceded only by heart disease, cancer and stroke (Schenk, D B; Rydel, R E; May, P; Little, S; Panetta, J; Lieberburg, I; Sinha, S. J Med Chem, 1995, 38: 4141-54). AD affects individuals in all races and ethnic groups, occurring slightly more commonly in females than males.

There is no remission in the progression of Alzheimer's disease, nor are there any disease-stabilizing drugs currently available (Selkoe, D J; Schenk, D. Annu Rev Pharmacol Toxicol, 2003, 43: 545-84). As such, onset of the disease is inevitably followed by increasing mental and physical incapacitation, loss of independent living, institutionalization and death. There is usually an 8-10 year period from symptom onset until death, but patients can survive for 20 years or more after the initial diagnosis of AD is made (Siegel).

Accordingly, there exists a need in the art for an agent which can be used for the treatment of Alzheimer's disease and other protein folding disorders.

All documents referred to herein are incorporated by reference in their entireties for all purposes.

### OBJECTS AND SUMMARY OF THE INVENTION

It is an object of the present invention to provide compounds and methods for treating protein folding disorders.

It is an object of certain embodiments of the present invention to provide compounds and methods for treating neurodegenerative diseases such as, e.g., Alzheimer's disease, tauopathies, cerebral amyloid angiopathy, Lewy body diseases (e.g. Parkinson's disease), dementia, tauopathies, cereberal amyloid angiopathies, Huntington's disease and prion-based spongiform encephalopathy.

It is an object of certain embodiments of the present invention to provide compounds and methods for treating systemic amyloidoses such as, e.g., secondary systemic amyloidosis, particularly those affecting the peripheral nerves, spleen, kidney, heart, intestine, smooth muscle or pancreas.

It is an object of the present invention to provide pharmaceutical compositions comprising an effective amount of a compound for treating protein folding disorders.

It is an object of certain embodiments of the present invention to provide pharmaceutical compositions comprising an effective amount of a compound for treating neurodegenerative diseases such as, e.g., Alzheimer's disease, tauopathies, cerebral amyloid angiopathy, Lewy body diseases (e.g. Parkinson's disease), dementia, Huntington's disease, prion-based spongiform encephalopathy and a combination thereof.

It is an object of certain embodiments of the present invention to provide pharmaceutical compositions comprising an effective amount of a compound for treating systemic amyloidoses, particularly those affecting the peripheral nerves, spleen, kidney, heart, intestine, smooth muscle or pancreas.

It is an object of certain embodiments of the present invention to provide compounds, methods and pharmaceutical compositions for inhibiting tau protein aggregation in a subject or patient.

Other objects and advantages of the present invention will become apparent from the disclosure herein.

In certain embodiments, the present invention is directed to a method for treating a protein folding disorder comprising administering an effective amount of a compound of formula (I) to a patient in need thereof:

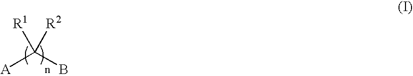

wherein A and B are independently a mono-, bi- or tri-cyclic aromatic or heteroaromatic substituent; wherein n=0 or 1; wherein, when n=1, R¹ and R² are independently hydrogen, alkyl, cycloalkyl, alkoxy, hydroxy, halogen, aryl, or together represent the group ═O; wherein A is substituted by A¹₍ₓ₎ and B is substituted by B¹_{(y)}; wherein x and y are independently an integer from 0 to 4; and

A¹₍ₓ₎ and B¹_{(y)} are each independently, for each value of x and y, selected from the group consisting of alkyl, alkenyl, alkynyl, cycloalkyl, cycloalkenyl, cycloalkynyl, aryl, alkylcarbonyl, alkoxy, trihalomethoxy, aryloxy, arylcarbonyl; alkoxycarbonyl, aryloxycarbonyl, amino, hydroxy, thio, thioether, cyano, nitro, halogen, carboxylic acid and sulfonic acid;

or a pharmaceutically acceptable salt thereof.

In certain preferred embodiments, A¹₍ₓ₎ and B¹_{(y)} are each independently, for each value of x and y, selected from the group consisting of alkyl, alkenyl, alkynyl, cycloalkyl, cycloalkenyl, cycloalkynyl, aryl, alkylcarbonyl, alkoxy, trihalomethoxy, aryloxy, arylcarbonyl; alkoxycarbonyl, aryloxycarbonyl, amino, hydroxy, thio, thioether, cyano, nitro, halogen or a pharmaceutically acceptable salt thereof.

In certain embodiments, the invention is directed to a method for treating a protein folding disorder comprising administering a compound of formula (I) to a subject wherein the subject is treated for the protein folding disorder.

In certain embodiments of the disclosed method, A and B of formula (I) are independently selected from the group consisting of phenyl, pyridyl, pyrrolyl, thiophenyl, furanyl, triazolyl, indolyl, naphthyl, benzofuranyl, quinolinyl, isoquinolinyl, benzothiophenyl, benzooxazolyl and benzimidazolyl.

In certain embodiments of the disclosed method, at least one of A and B of formula (I) are indolyl and in certain embodiments, both of A and B of formula (I) are indolyl.

In certain embodiments of the disclosed method, the compound of formula (I) is:

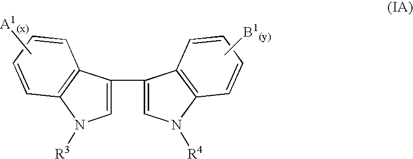

In certain embodiments of the disclosed method, the compound of formula (I) is:

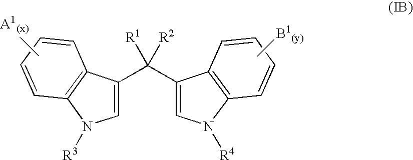

In certain embodiments of the disclosed method, the compound of formula (I) is:

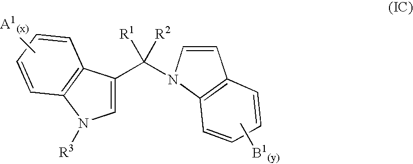

In certain embodiments of the disclosed method, x is 1 and A¹ is at the 5, 6 or 7 position.

In certain embodiments of the disclosed method, x is 1 and A¹ is CO₂H.

In certain embodiments of the disclosed method, x is 1 and A¹ is at the 5 position; wherein R¹ and R² are independently hydrogen, alkyl, cycloalkyl, alkoxy, hydroxy, halogen, or aryl.

In certain embodiments of the disclosed method, x is 1 and A¹ is CO₂H and is at the 5 position.

In certain embodiments of the disclosed method, x is 1 and A¹ is at the 5 position; wherein R¹ and R² are independently hydrogen, alkyl, alkoxy, hydroxy or halogen.

In certain embodiments of the disclosed method, R³ and R⁴ are independently hydrogen, alkyl, alkenyl, alkynyl, cycloalkyl, cycloalkenyl, cycloalkynyl, aryl, arylalkyl, alkylcarbonyl, arylcarbonyl; alkoxycarbonyl, aryloxycarbonyl, arylsulfonyl or alkylsulfonyl.

In certain embodiments of the disclosed method, x is 1 and A¹ is CO₂H and is at the 6 position.

In certain embodiments of the disclosed method, A¹ is at the 5 position.

In certain embodiments, A¹ is hydroxy.

In certain embodiments of the disclosed method, A¹ is selected from the group consisting of halogen, hydroxy, alkyl, alkoxy, aryl and heteroaryl.

In certain embodiments of the disclosed method, in B¹ is at the 5 or 6 position.

In certain embodiments of the disclosed method, B¹ is selected from the group consisting of halogen, hydroxy, alkyl, alkoxy, aryl, thio, thioether, and trihalomethoxy.

In certain embodiments of the disclosed method, B¹ is at the 5 position.

In certain embodiments of the disclosed method, B¹ is selected from the group consisting of halogen, hydroxy, alkyl, alkoxy, aryl and heteroaryl.

In certain embodiments of the disclosed method, B¹ is at the 7 position.

In certain embodiments of the disclosed method, y is 1 and B¹ is at the 5 position; wherein R¹ and R² are independently hydrogen, alkyl, cycloalkyl, alkoxy, hydroxy, halogen, or aryl.

In certain embodiments of the disclosed method, y is 1 and B¹ is CO₂H.

In certain embodiments of the disclosed method, n is an integer from 1 to 10; wherein, when n is not 0, R¹ and R² are substituted on one or more carbons and is as described above.

In certain embodiments of the disclosed method, the compound of formula (I) is selected from the group consisting of:
3,3′-bi-indolyl;
5-methoxy-3-(5-methoxyindol-3-yl)-indole;
3-(5-bromoindol-3-yl)-indole-5-carboxylic acid;
3-(indol-3-yl)-indole-5-carboxylic acid;
3-(5-methoxyindol-3-yl)-indole-5-carboxylic acid;
3-(5-fluoroindol-3-yl)-indole-5-carboxylic acid;
3-(5-chloroindol-3-yl)-indole-5-carboxylic acid;
3-(5-methylindol-3-yl)-indole-5-carboxylic acid;
3-(5-(trifluoromethoxy)-indol-3-yl)indole-5-carboxylic acid;
3-(5-hydroxyindol-3-yl)-indole-5-carboxylic acid;
3-(indol-3-yl)-indole-6-carboxylic acid;
3-(indol-3-yl)-indole-7-carboxylic acid;
3-(5-hydroxyindol-3-yl)-indol-5-ol; and

a pharmaceutically acceptable salt thereof.

In certain embodiments of the disclosed method, the compound of formula (I) is selected from the group consisting of:
di-(indol-3-yl)methane;
3-((indol-1-yl)methyl)-indole;
bis-(5-methoxy-indol-3-yl)methane;
5-methoxy-3-((5-methoxy-indol-1-yl)methyl)-indole;
3-((indol-3-yl)methyl)-indole-5-carboxylic acid;
bis-(indole-5-carboxylic acid-3-yl)methane;
bis-(5-hydroxy-indol-3-yl)methane;
1,2-di-(indol-3-yl)ethane;
3-(2-(5-bromo-indol-3-yl)ethyl)-indole-5-carboxylic acid;
1,2-bis-(indole-5-carboxylic acid-3-yl)ethane; and

a pharmaceutically acceptable salt thereof.

In certain embodiments the present invention is directed to a method for treating a protein folding disorder comprising administering an effective amount of a compound of formula (II) to a patient in need thereof:

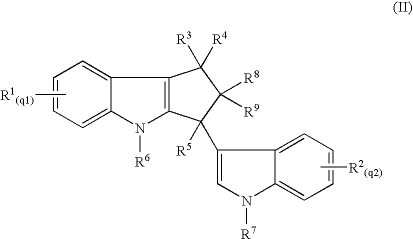

wherein q₁ and q₂ are each independently selected from an integer from 0 to 4;

wherein each R¹ and each R² is independently selected from the group consisting of alkyl, alkenyl, alkynyl, cycloalkyl, cycloalkenyl, cycloalkynyl, aryl, alkylcarbonyl, alkoxy, cycloalkyloxy, trihalomethoxy, aryloxy, arylcarbonyl, alkoxycarbonyl, amino, hydroxy, thioether, cyano, nitro, halogen, and carboxylic acid; and

wherein R³, R⁴, R⁵, R⁸ and R⁹ are independently selected from the group consisting of hydrogen, alkyl, alkenyl, alkynyl, cycloalkyl, cycloalkenyl, cycloalkynyl, aryl, alkylcarbonyl, alkoxy, cycloalkyloxy, trihalomethoxy, aryloxy, arylcarbonyl, alkoxycarbonyl, amino, hydroxy, thioether, cyano, nitro, halogen, and carboxylic acid; and

wherein R⁶ and R⁷ are independently selected from the group consisting of hydrogen, alkyl, alkenyl, alkynyl, cycloalkyl, cycloalkenyl, cycloalkynyl, aryl, arylalkyl, alkylcarbonyl, arylcarbonyl; alkoxycarbonyl, aryloxycarbonyl, arylsulfonyl, alkylsulfonyl;

or pharmaceutically acceptable salts thereof.

In certain preferred embodiments, R¹ and R² are the same and are hydroxy.

In certain preferred embodiments, R³, R⁴, and R⁵ are each alkyl.

In certain embodiments the present invention is directed to a method for treating a protein folding disorder comprising administering an effective amount of a compound of formula (III) to a patient in need thereof:

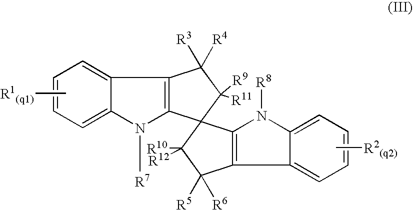

wherein q₁ and q₂ are each independently selected from an integer from 0 to 4;

wherein each R¹ and each R² is independently selected from the group consisting of alkyl, alkenyl, alkynyl, cycloalkyl, cycloalkenyl, cycloalkynyl, aryl, alkylcarbonyl, alkoxy, cycloalkyloxy, trihalomethoxy, aryloxy, arylcarbonyl, alkoxycarbonyl, amino, hydroxy, thioether, cyano, nitro, halogen, and carboxylic acid; and

wherein R³, R⁴, R⁵, R⁶, R⁹, R¹⁰, R¹¹ and R¹² are independently selected from the group consisting of hydrogen, alkyl, alkenyl, alkynyl, cycloalkyl, cycloalkenyl, cycloalkynyl, aryl, alkylcarbonyl, alkoxy, cycloalkyloxy, trihalomethoxy, aryloxy, arylcarbonyl, alkoxycarbonyl, amino, hydroxy, thioether, cyano, nitro, halogen, and carboxylic acid; and

wherein R⁷ and R⁸ are independently hydrogen, alkyl, alkenyl, alkynyl, cycloalkyl, cycloalkenyl, cycloalkynyl, aryl, arylalkyl, alkylcarbonyl, arylcarbonyl; alkoxycarbonyl, aryloxycarbonyl, arylsulfonyl, alkylsulfonyl;

or pharmaceutically acceptable salts thereof.

In certain preferred embodiments, R¹ and R² are the same and are hydroxy.

In certain preferred embodiments, R³, R⁴, R⁵, and R⁶ are each alkyl.

In certain embodiments the present invention is directed to a method for treating a protein folding disorder comprising administering an effective amount of a compound of formula (IV) to a patient in need thereof:

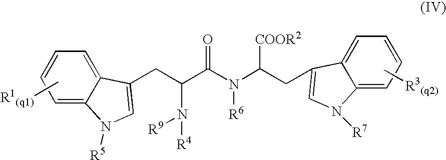

wherein q₁ and q₂ are each independently selected from an integer from 0 to 4;

wherein each R¹ and each R³ are independently selected from the group consisting of alkyl, alkenyl, alkynyl, cycloalkyl, cycloalkenyl, cycloalkynyl, aryl, alkylcarbonyl, alkoxy, cycloalkoxy, trihalomethoxy, aryloxy, arylcarbonyl, alkoxycarbonyl, amino, hydroxy, thioether, cyano, nitro, halogen, and carboxylic acid;

wherein R² is selected from the group consisting of hydrogen, alkyl, alkenyl, alkynyl, cycloalkyl, cycloalkenyl and cycloalkynyl;

wherein R⁴ and R⁹ are independently selected from the group consisting of hydrogen, alkyl, alkenyl, alkynyl, cycloalkyl, cycloalkenyl, cycloalkynyl and —CO₂—R⁸; wherein R⁸ is selected from the group consisting of hydrogen, alkyl, alkenyl, alkynyl, cycloalkyl, cycloalkenyl, cycloalkynyl, aryl, alkylcarbonyl, arylalkyl, arylcarbonyl, alkoxycarbonyl, amino; and

wherein R⁵, R⁶ and R⁷ are independently hydrogen, alkyl, alkenyl, alkynyl, cycloalkyl, cycloalkenyl, cycloalkynyl, aryl, arylalkyl, alkylcarbonyl, arylcarbonyl; alkoxycarbonyl, aryloxycarbonyl, arylsulfonyl, alkylsulfonyl; or

pharmaceutically acceptable salts thereof, isomers, stereoisomers, or diastereomers thereof.

In certain preferred embodiments, R⁸ is benzyl.

In certain preferred embodiments, R⁴ is hydrogen or carbobenzyloxy.

In certain embodiments, the invention is directed to a method for treating a protein folding disorder comprising administering a compound of formula (IV) to a subject wherein the subject is treated for the protein folding disorder.

In certain embodiments, of the disclosed method, the compound of formula (IV) is selected from the group consisting of

CBZ-L-Trp-L-Trp-OH;

CBZ-L-Trp-D-Trp-OH;

CBZ-D-Trp-L-Trp-OH;

CBZ-D-Trp-D-Trp-OH;

H-L-Trp-L-Trp-OH;

H-L-Trp-D-Trp-OH;

H-D-Trp-L-Trp-OH;

H-D-Trp-D-Trp-OH;

and pharmaceutically acceptable salts thereof.

For purposes of the present invention “CBZ” means carbobenzyloxy.

In certain embodiments the present invention is directed to a method for treating a protein folding disorder comprising administering an effective amount a compound of formula (V) to a patient in need thereof:

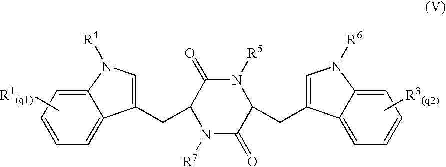

wherein q₁ and q₂ are each independently selected from an integer from 0 to 4;

wherein each R¹ and R³ are independently selected from the group consisting of alkyl, alkenyl, alkynyl, cycloalkyl, cycloalkenyl, cycloalkynyl, aryl, alkylcarbonyl, alkoxy, cycloalkoxy, trihalomethoxy, aryloxy, arylcarbonyl, alkoxycarbonyl, amino, hydroxy, thioether, cyano, nitro, halogen, and carboxylic acid; and

wherein R⁴, R⁵, R⁶ and R⁷ are independently hydrogen, alkyl, alkenyl, alkynyl, cycloalkyl, cycloalkenyl, cycloalkynyl, aryl, arylalkyl, alkylcarbonyl, arylcarbonyl; alkoxycarbonyl, aryloxycarbonyl, arylsulfonyl, alkylsulfonyl; or pharmaceutically acceptable salts thereof, isomers, stereoisomers, or diastereomers thereof.

In certain embodiments, the invention is directed to a method for treating a protein folding disorder comprising administering a compound of formula (V) to a subject wherein the subject is treated for the protein folding disorder.

In certain embodiments, of the disclosed method, the compound of formula (V) is selected from the group consisting of

Cyclo(L-Trp-L-Trp);

Meso-cyclo(Trp-Trp);

Cyclo(D-Trp-D-Trp); and

pharmaceutically acceptable salts thereof.

In certain embodiments, the present invention is directed to a method for treating a protein folding disorder comprising administering an effective amount of a compound of formula (VI) to a patient in need thereof:

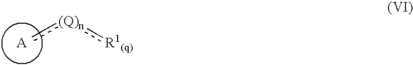

wherein A is a mono-, bicyclic, or tricyclic aromatic or heteroaromatic ring structure;

Q is —C—, —CH—, or —CH₂—

n is an integer from 0 to 4;

q is an integer from 1 to 3;

R¹ is alkenyl, alkynyl, cycloalkyl, cycloalkenyl, cycloalkynyl, alkylcarbonyl, alkoxy, cycloalkyloxy, trihalomethoxy, aryloxy, arylcarbonyl, alkoxycarbonyl, amino, hydroxy, thioether, cyano, nitro, halogen, carboxylic acid, a mono-, bicyclic, tricyclic aromatic or heteroaromatic ring; wherein when R¹ is a mono-, bicyclic, tricyclic aromatic or heteroaromatic ring, then R¹ is optionally further substituted with one or more R² groups each independently selected from the group consisting of alkenyl, alkynyl, cycloalkyl, cycloalkenyl, cycloalkynyl, aryl, alkylcarbonyl, alkoxy, hydroxy, cycloalkyloxy, trihalomethoxy, aryloxy, arylcarbonyl, alkoxy;

wherein A is further optionally substituted with one or more R³ groups independently selected from hydrogen, alkyl, alkenyl, alkynyl, cycloalkyl, cycloalkenyl, cycloalkynyl, aryl, alkylcarbonyl, alkoxy, cycloalkyloxy, trihalomethoxy, aryloxy, arylcarbonyl, alkoxycarbonyl, amino, hydroxy, thioether, cyano, nitro, halogen, and carboxy; or

pharmaceutically acceptable salts thereof; and

wherein Formula (VI) is not an unsubstituted or substituted indole-3-propionic acid.

In certain embodiments, A is selected from the group consisting of indolyl, phenyl, pyridyl, pyrrolyl, thiophenyl, furanyl, tetrazolyl, naphthyl, benzofuranyl, quinolinyl, and isoquinolyl.

In certain embodiments the present invention is directed to a method for treating a protein folding disorder comprising administering an effective amount of a compound of formula (VII) to a patient in need thereof:

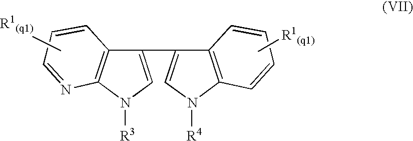

wherein q₁ and q₂ are each independently selected from an integer from 0 to 4;

wherein each R¹ and each R² is independently selected from the group consisting of alkyl, alkenyl, alkynyl, cycloalkyl, cycloalkenyl, cycloalkynyl, aryl, alkylcarbonyl, alkoxy, cycloalkyloxy, trihalomethoxy, aryloxy, arylcarbonyl, alkoxycarbonyl, amino, hydroxy, methoxy, thioether, cyano, nitro, halogen, and carboxylic acid; and

wherein R³ and R⁴ are independently selected from the group consisting of hydrogen, alkyl, alkenyl, alkynyl, cycloalkyl, cycloalkenyl, cycloalkynyl, aryl, arylalkyl, alkylcarbonyl, arylcarbonyl; alkoxycarbonyl, aryloxycarbonyl, arylsulfonyl or alkylsulfonyl;

or pharmaceutically acceptable salts thereof.

In certain preferred embodiments, the 2,3 bond of aza-indole is reduced.

In certain embodiments, the compounds are borane adducts at N-7 of the aza-indole.

In certain preferred embodiments, the compound of formula (VII) is selected from the group consisting of 3-(5-methoxy-indol-3-yl)-7-azaindole; 3-(5-bromo-indol-3-yl)-7-aza-indole; 3-(7-aza-indol-3-yl)-indol-5-ol; 3-(2,3-dihydro-7-aza-indol-3-yl)-indol-5-ol; 3-(7-aza-indol-3-yl)-indole-5-carboxylic acid; and pharmaceutically acceptable salts thereof.

In certain embodiments the present invention is directed to a method for treating a protein folding disorder comprising administering an effective amount of a compound of formula (VIII) to a patient in need thereof:

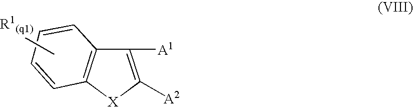

wherein A¹ and A² are independently selected from the group consisting of hydrogen, any substituted or non-substituted aromatic ring, carboxylic acid; and pharmaceutically acceptable salts thereof;

wherein X is selected from the group consisting of oxygen, sulfur or N—R², where R² is selected from the group consisting of hydrogen, alkyl, aryl, sulfonylaryl, t-butoxycarbonyl (tBOC); and

wherein R¹ is alkyl, alkenyl, alkynyl, cycloalkyl, cycloalkenyl, cycloalkynyl, aryl, alkylcarbonyl, alkoxy, cycloalkyloxy, trihalomethoxy, aryloxy, arylcarbonyl, alkoxycarbonyl, amino, hydroxy, methoxy, thioether, cyano, nitro, halogen, carboxylic acid; and pharmaceutically acceptable salts thereof.

In certain preferred embodiments, the compound of formula (VIII) is selected from the group consisting of 2,3-bis(4-methoxybenzyl)-indole-5-carboxylic acid; 2,3-bis(4-hydroxybenzyl)-indole-5-carboxylic acid; 3-(4-hydroxybenzyl)-indole-5-carboxylic acid; and pharmaceutically acceptable salts thereof.

In certain embodiments, the invention is directed to compounds of formula (I), (II), (III), (IV), (V), (VI), (VII) or (VIII).

In certain embodiments, the present invention is directed to a compound of formula (IX):
A-B (IX)

wherein A and B are indolyl substituents; wherein A is substituted by A¹₍ₓ₎ and B is substituted by B¹_{(y)}; wherein x and y are independently an integer from 0 to 3 and the sum of x and y is from 1 to 3; and

each A¹ and each B¹ are independently selected from the group consisting of alkyl, alkenyl, alkynyl, cycloalkyl, cycloalkenyl, cycloalkynyl, aryl, alkylcarbonyl, alkoxy, trihalomethoxy, aryloxy, arylcarbonyl; alkoxycarbonyl, aryloxycarbonyl, amino, hydroxy, thio, thioether, cyano, nitro, halogen, carboxylic acid and sulfonic acid;

or a pharmaceutically acceptable salt thereof.

In certain embodiments, the present invention is directed to a compound of formula (IX):
A-B (IX)

wherein A and B are indolyl substituents; wherein A is substituted by A¹₍ₓ₎ and B is substituted by B¹_{(y)}; wherein x and y are independently an integer from 0 to 4 and the sum of x and y is at least 1;

each A¹ and each B¹ are independently selected from the group consisting of alkyl, alkenyl, alkynyl, cycloalkyl, cycloalkenyl, cycloalkynyl, aryl, alkylcarbonyl, alkoxy, trihalomethoxy, aryloxy, arylcarbonyl; alkoxycarbonyl, aryloxycarbonyl, amino, thio, thioether, cyano, nitro, halogen, carboxylic acid and sulfonic acid;

or a pharmaceutically acceptable salt thereof.

In certain embodiments, the present invention is directed to a compound of formula (IX):
A-B (IX)

wherein A and B are indolyl substituents; wherein A is substituted by A¹₍ₓ₎ and B is substituted by B¹_{(y)}; wherein x and y are independently an integer from 0 to 4 and the sum of x and y is at least 1;

each A¹ and each B¹ are independently selected from the group consisting of alkyl, alkenyl, alkynyl, cycloalkyl, cycloalkenyl, cycloalkynyl, aryl, alkylcarbonyl, alkoxy, trihalomethoxy, aryloxy, arylcarbonyl; alkoxycarbonyl, aryloxycarbonyl, amino, hydroxy, thio, thioether, cyano, nitro, halogen, carboxylic acid and sulfonic acid; provided that the total number of hydroxy substituents is less than 4;

or a pharmaceutically acceptable salt thereof.

In certain embodiments of the present invention, the point of attachment for at least one of A and B is at the 1, 2 or 3 position of the indolyl.

In certain embodiments of the present invention, the point of attachment for both A and B is at the 1, 2 or 3 position of the indolyl.

In certain embodiments of the present invention, A is substituted by A¹₍ₓ₎ in at least one of the 4, 5, 6 or 7 positions.

In certain embodiments of the present invention, B is substituted by B¹_{(y)} in at least one of the 4, 5, 6 or 7 positions.

In certain embodiments of the present invention, the compound of formula (IX) is:

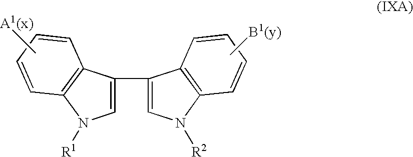

In certain embodiments of the present invention, x is 1 and A¹ is at the 5 position.

In certain embodiments of the present invention, y is 1 and B¹ is CO₂H.

In certain embodiments of the present invention, B¹ is at the 5, 6 or 7 position.

In certain embodiments of the present invention, A¹ is selected from the group consisting of halogen, OC₁₋₃ alkyl and OC(halogen)₃.

In certain embodiments of the present invention, x is 0, y is 1 and B¹ is CO₂H at the 5, 6 or 7 position.

In certain embodiemtns of the present invention, R¹ and R² are independently selected from the group consisting of hydrogen, alkyl, alkenyl, alkynyl, cycloalkyl, cycloalkenyl, cycloalkynyl, aryl, arylalkyl, alkylcarbonyl, arylcarbonyl; alkoxycarbonyl, aryloxycarbonyl, arylsulfonyl and alkylsulfonyl.

In certain embodiments, the present invention is directed to a pharmaceutical composition comprising an effective amount of a compound of formula (IX):
A-B (IX)

wherein A and B are indolyl substituents; wherein A is substituted by A¹₍ₓ₎ and B is substituted by B¹_{(y)}; wherein x and y are independently an integer from 0 to 4; and

each A¹ and each B¹ are independently selected from the group consisting of alkyl, alkenyl, alkynyl, cycloalkyl, cycloalkenyl, cycloalkynyl, aryl, alkylcarbonyl, alkoxy, trihalomethoxy, aryloxy, arylcarbonyl; alkoxycarbonyl, aryloxycarbonyl, amino, hydroxy, thio, thioether, cyano, nitro, halogen, carboxylic acid and sulfonic acid;

or a pharmaceutically acceptable salt thereof to treat a protein folding disorder.

In certain embodiments, the present invention is directed to a pharmaceutical composition comprising an effective amount of a compound of formula (IX):
A-B (IX)

wherein A and B are indolyl substituents; wherein A is substituted by A¹₍ₓ₎ and B is substituted by B¹_{(y)}; wherein x and y are independently an integer from 0 to 4; and

each A¹ and each B¹ are independently selected from the group consisting of alkyl, alkenyl, alkynyl, cycloalkyl, cycloalkenyl, cycloalkynyl, aryl, alkylcarbonyl, alkoxy, trihalomethoxy, aryloxy, arylcarbonyl; alkoxycarbonyl, aryloxycarbonyl, amino, hydroxy, thio, thioether, cyano, nitro, halogen, carboxylic acid and sulfonic acid;

or a pharmaceutically acceptable salt thereof;

to treat a protein folding disorder, e.g., a neurodegenerative disease such as Alzheimer's disease, tauopathies, cerebral amyloid angiopathy, Lewy body diseases (e.g. Parkinson's disease), dementia, Huntington's disease and prion-based spongiform encephalopathy, and a combination thereof.

In certain embodiments, the present invention is directed to a compound of formula (X):

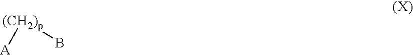

wherein A and B are indolyl substituents; wherein A is substituted by A¹₍ₓ₎ and B is substituted by B¹_{(y)}; wherein x and y are independently an integer from 0 to 4 and the sum of x and y is at least 1, provided that when x and y both equal 1, A¹ and B¹ are not both CO₂H and are not both halogen;

p is 1 or 2; and

each A¹ and each B¹ are independently selected from the group consisting of alkyl, alkenyl, alkynyl, cycloalkyl, cycloalkenyl, cycloalkynyl, aryl, alkylcarbonyl, alkoxy, trihalomethoxy, aryloxy, arylcarbonyl; alkoxycarbonyl, aryloxycarbonyl, amino, hydroxy, thio, thioether, cyano, nitro, halogen, carboxylic acid and sulfonic acid; or a pharmaceutically acceptable salt thereof.

In certain embodiments, the present invention is directed to a compound of formula (X):

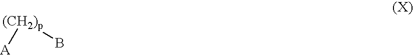

wherein A and B are indolyl substituents; wherein A is substituted by A¹₍ₓ₎ and B is substituted by B¹_{(y)}; wherein x and y are independently an integer from 0 to 4 and the sum of x and y is at least 1;

p is 1 or 2; and

each A¹ and each B¹ are independently selected from the group consisting of alkyl, alkenyl, alkynyl, cycloalkyl, cycloalkenyl, cycloalkynyl, aryl, alkylcarbonyl, alkoxy, trihalomethoxy, aryloxy, arylcarbonyl; alkoxycarbonyl, aryloxycarbonyl, amino, hydroxy, thio, thioether, cyano, nitro, and sulfonic acid;

or a pharmaceutically acceptable salt thereof.

In certain embodiments, the present invention is directed to a compound of formula (X):

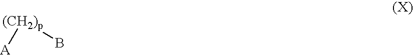

wherein A and B are indolyl substituents; wherein A is substituted by A¹₍ₓ₎ and B is substituted by B¹_{(y)}; wherein x and y are independently an integer from 0 to 4 and the sum of x and y is at least 1;

p is 1 or 2; and

each A¹ and each B¹ are independently selected from the group consisting of alkyl, alkenyl, alkynyl, cycloalkyl, cycloalkenyl, cycloalkynyl, aryl, alkylcarbonyl, alkoxy, trihalomethoxy, aryloxy, arylcarbonyl; alkoxycarbonyl, aryloxycarbonyl, amino, hydroxy, thio, thioether, cyano, nitro, halogen, carboxylic acid and sulfonic acid; provided that the total number of CO₂H substituents is not more than 1 and the total number of halogen substituents is not more than 1;

or a pharmaceutically acceptable salt thereof.

In certain embodiments, the present invention is directed to a compound of formula (X):

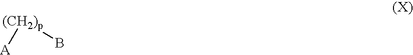

wherein A and B are indolyl substituents; wherein A is substituted by A¹₍ₓ₎ and B is substituted by B¹_{(y)}; wherein x and y are independently an integer from 0 to 4 and the sum of x and y is at least 1;

p is 2; and

each A¹ and each B¹ are independently selected from the group consisting of alkyl, alkenyl, alkynyl, cycloalkyl, cycloalkenyl, cycloalkynyl, aryl, alkylcarbonyl, alkoxy, trihalomethoxy, aryloxy, arylcarbonyl; alkoxycarbonyl, aryloxycarbonyl, amino, hydroxy, thio, thioether, cyano, nitro, halogen, carboxylic acid and sulfonic acid;

or a pharmaceutically acceptable salt thereof.

In certain embodiments of the present invention, the point of attachment for at least one of A and B is at the 1, 2 or 3 position of the indolyl.

In certain embodiments of the present invention, the point of attachment for both A and B is at the 1, 2 or 3 position of the indolyl.

In certain embodiments of the present invention, A is substituted by A¹₍ₓ₎ in at least one of the 4, 5, 6 or 7 positions of the indolyl.

In certain embodiments of the present invention, B is substituted by B¹_{(y)} in at least one of the 4, 5, 6 or 7 positions of the indolyl.

In certain embodiments of the present invention, the compound of formula (X) is:

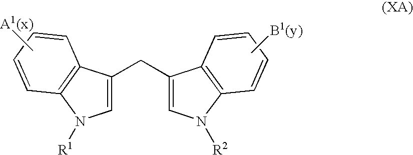

In certain embodiments of the present invention, the compound of formula (X) is:

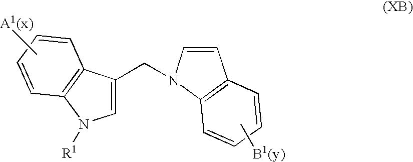

In certain embodiments of the present invention, x is 1 and A¹ is at the 5 position.

In certain embodiments of the present invention, y is 1 and B¹ is at the 6 position.

In certain embodiments of the present invention, y is 1 and B¹ is at the 5 position.

In certain embodiments of the present invention, A¹ is selected from the group consisting of halogen, OC₁₋₃ alkyl, hydroxy and CO₂H.

In certain embodiments of the present invention, B¹ is selected from the group consisting of OC₁₋₃ alkyl, hydroxy and CO₂H.

In certain embodiments of the present invention, A¹ is selected from the group consisting of halogen and CO₂H.

In certain embodiments of the present invention, R¹ and R² are independently selected from the group consisting of hydrogen, alkyl, alkenyl, alkynyl, cycloalkyl, cycloalkenyl, cycloalkynyl, aryl, arylalkyl, alkylcarbonyl, arylcarbonyl; alkoxycarbonyl, aryloxycarbonyl, arylsulfonyl and alkylsulfonyl.

In certain embodiments of the present invention, B¹ is selected from the group consisting of hydroxyl and CO₂H.

In certain embodiments, the present invention is directed to a pharmaceutical composition comprising an effective amount of a compound of formula (X):

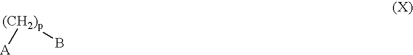

wherein A and B are indolyl substituents; wherein A is substituted by A¹₍ₓ₎ and B is substituted by B¹_{(y)}; wherein x and y are independently an integer from 0 to 4;

p is 1 or 2; and

each A¹ and each B¹ are independently selected from the group consisting of alkyl, alkenyl, alkynyl, cycloalkyl, cycloalkenyl, cycloalkynyl, aryl, alkylcarbonyl, alkoxy, trihalomethoxy, aryloxy, arylcarbonyl; alkoxycarbonyl, aryloxycarbonyl, amino, hydroxyl, thio, thioether, cyano, nitro, halogen, carboxylic acid and sulfonic acid;

or a pharmaceutically acceptable salt thereof to treat a protein folding disorder, e.g., a neurodegenerative disease such as Alzheimer's disease, tauopathies, cerebral amyloid angiopathy, Lewy body diseases (e.g. Parkinson's disease), dementia, Huntington's disease and prion-based spongiform encephalopathy and a combination thereof.

In certain embodiments, the invention is directed to a method of treating a protein folding disorder comprising administering to a patient in need thereof an effective amount of a compound which attenuates the increase in thioflavin T fluorescence by greater than 30%; greater than 60%; or greater than 90%; relative to beta-amyloid with vehicle as a control, at 20 hours when subjected to a beta-amyloid thioflavin T aggregation assay. In certain such embodiments, the compound is a compound of formula (I), (II), (III), (IV), (V), (VI), (VII), (VIII), (IX) or (X).

In certain embodiments, the invention is directed to a method of treating a protein folding disorder comprising administering to a patient in need thereof an effective amount of a compound which attenuates the increase in thioflavin T fluorescence by greater than 30%; greater than 60%; or greater than 90%; relative to beta-amyloid with vehicle as a control, at 30 hours when subjected to a beta-amyloid thioflavin T aggregation assay. In certain such embodiments, the compound is a compound of formula (I), (II), (III), (IV), (V), (VI), (VII), (VIII), (IX) or (X).

In certain embodiments, the invention is directed to a method of treating a protein folding disorder comprising administering to a patient in need thereof an effective amount of a compound which attenuates the increase in thioflavin S (ThS) fluorescence by greater than 30%; greater than 60%; or greater than 90%; relative to tau with vehicle as a control, at 30 hours when subjected to a tau thioflavin S (ThS) aggregation assay. In certain such embodiments, the compound is a compound of formula (I), (II), (III), (IV), (V), (VI), (VII), (VIII), (IX) or (X).

In certain embodiments, the invention is directed to a method of treating a protein folding disorder comprising administering to a patient in need thereof an effective amount of a compound which attenuates the increase in thioflavin T (ThT) fluorescence by greater than 30%; greater than 60%; or greater than 90%; relative to alpha-synuclein with vehicle as a control, at 30 hours when subjected to an alpha-synuclein thioflavin T (ThT) aggregation assay. In certain such embodiments, the compound is a compound of formula (I), (II), (III), (IV), (V), (VI), (VII), (VIII), (IX) or (X).

In certain embodiments, the invention is directed to a method of treating a protein folding disorder comprising administering to a subject in need thereof an effective amount of a compound which, when co-incubated with beta-amyloid, causes the peptide to exhibit circular dichroism, at 193 nm after 48 hours, of less than that of beta amyloid with vehicle. In certain such embodiments, the compound is a compound of formula (I), (II), (III), (IV), (V), (VI), (VII), (VIII), (IX) or (X).

In certain embodiments, the invention is directed to a method of treating a protein folding disorder comprising administering to a subject in need thereof an effective amount of a compound which, when co-incubated with beta-amyloid, causes the peptide to exhibit circular dichroism, at 193 nm after 48 hours, of at least 2 mdeg less than that of beta amyloid with vehicle. In certain such embodiments, the compound is a compound of formula ((I), (II), (III), (IV), (V), (VI), (VII), (VIII), (IX) or (X).

In certain embodiments, the invention is directed to a method of treating a protein folding disorder comprising administering to a subject in need thereof an effective amount of a compound which, when co-incubated with beta-amyloid, causes the peptide to exhibit circular dichroism, at 193 nm after 72 hours, of less than that of beta amyloid with vehicle. In certain such embodiments, the compound is a compound of formula (I), (II), (III), (IV), (V), (VI), (VII), (VIII), (IX) or (X).

In certain embodiments, the invention is directed to a method of treating a protein folding disorder comprising administering to a subject in need thereof an effective amount of a compound which, when co-incubated with beta-amyloid, causes the peptide to exhibit circular dichroism, at 193 nm after 72 hours, of at least 2 mdeg less than that of beta amyloid with vehicle. In certain such embodiments, the compound is a compound of formula (I), (II), (III), (IV), (V), (VI), (VII), (VIII), (IX) or (X).

In certain embodiments, the invention is directed to a method of treating a protein folding disorder comprising administering to a subject in need thereof an effective amount of a compound which, when co-incubated with beta-amyloid, causes the peptide to exhibit circular dichroism, at 193 nm after 72 hours, of at least 5 mdeg less than that of beta amyloid with vehicle. In certain such embodiments, the compound is a compound of formula (I), (II), (III), (IV), (V), (VI), (VII), (VIII), (IX) or (X).

In certain embodiments, the invention is directed to a method of treating a protein folding disorder comprising administering to a patient in need thereof, a compound whose aromatic or heteroaromatic substituents each exhibit sufficient gas-phase cation-π binding energy to cationic residues of the protein to treat the protein folding disorder. In certain such embodiments, the compound is a compound of formula (I), (II), (III), (IV), (V), (VI), (VII), (VIII), (IX) or (X).

In certain embodiments, the invention is directed to a method of treating a protein folding disorder comprising administering to a patient in need thereof, a compound whose aromatic or heteroaromatic substituents each exhibit a gas-phase cation-π binding energy to cationic residues of the protein of at least 15 kcal/mol in a RHF/6-31G(d)//RHF/3-21G optimization calculation, as implemented within the Gaussian98 computer program (Rev. A.9. 1998, Gaussian Inc., Pittsburgh, Pa., U.S.A., See Example 20). In certain such embodiments, the compound is a compound of formula (I), (II), (III), (IV), (V), (VI), (VII), (VIII), (IX) or (X).

In certain embodiments, the invention is directed to a method for inhibiting tau protein aggregation or for treating a protein folding disorder comprising administering a compound which attenuates the increase in thioflavin S fluorescence by greater than 30% at 20 hours, relative to tau441 with vehicle as a control, in a tau aggregation assay. In certain such embodiments, the compound is a compound of formula (I), (II), (III), (IV), (V), (VI), (VII), (VIII), (IX) or (X).

In certain embodiments, the invention is directed to a method for inhibiting tau protein aggregation or for treating a protein folding disorder comprising administering a compound which attenuates an increase in thioflavin S fluorescence by greater than 60% at 20 hours, relative to tau441 with vehicle as a control, in a tau aggregation assay. In certain such embodiments, the compound is a compound of formula (I), (II), (III), (IV), (V), (VI), (VII), (VIII), (IX) or (X).

In certain embodiments, the invention is directed to a method for inhibiting tau protein aggregation or for treating a protein folding disorder comprising administering a compound which attenuates an increase in thioflavin S fluorescence by greater than 90% at 20 hours, relative to tau441 with vehicle as a control, in a tau aggregation assay. In certain such embodiments, the compound is a compound of formula (I), (II), (III), (IV), (V), (VI), (VII), (VIII), (IX) or (X).

In certain embodiments, the invention is directed to a method for inhibiting tau protein aggregation or for treating a protein folding disorder comprising administering a compound which attenuates an increase in thioflavin S fluorescence by greater than 30% at 30 hours, relative to tau441 with vehicle as a control, in a tau aggregation assay. In certain such embodiments, the compound is a compound of formula (I), (II), (III), (IV), (V), (VI), (VII), (VIII), (IX) or (X).

In certain embodiments, the invention is directed to a method for inhibiting tau protein aggregation or for treating a protein folding disorder comprising administering a compound which attenuates an increase in thioflavin S fluorescence by greater than 60% at 30 hours, relative to tau441 with vehicle as a control, in a tau aggregation assay. In certain such embodiments, the compound is a compound of formula (I), (II), (III), (IV), (V), (VI), (VII), (VIII), (IX) or (X).

In certain embodiments, the invention is directed to a method for inhibiting tau protein aggregation or for treating a protein folding disorder comprising administering a compound which attenuates an increase in thioflavin S fluorescence by greater than 90% at 30 hours, relative to tau441 with vehicle as a control, in a tau aggregation assay. In certain such embodiments, the compound is a compound of formula (I), (II), (III), (IV), (V), (VI), (VII), (VIII), (IX) or (X).

In certain embodiments, the invention is directed to a method of treating a protein folding disorder comprising administering to a patient in need thereof an effective amount of a compound which:

(i) attenuates the increase in thioflavin T fluorescence by greater than 30%, relative to beta-amyloid with vehicle as a control, at 20 hours when subjected to a beta-amyloid thioflavin T aggregation assay, and

(ii) attenuates an increase in fluorescence by greater than 30%, greater than 60% or greater than 90% at 20 hours, relative to tau441 with vehicle as a control, in a tau thioflavin S aggregation assay.

In certain embodiments, the invention is directed to a method of treating a protein folding disorder comprising administering to a patient in need thereof an effective amount of a compound which:

(i) attenuates the increase in thioflavin T fluorescence by greater than 60%, relative to beta-amyloid with vehicle as a control, at 20 hours when subjected to a beta-amyloid thioflavin T aggregation assay, and

(ii) attenuates an increase in fluorescence by greater than 30%, greater than 60% or greater than 90% at 20 hours, relative to tau441 with vehicle as a control, in a tau thioflavin S aggregation assay.

In certain embodiments, the invention is directed to a method of treating a protein folding disorder comprising administering to a patient in need thereof an effective amount of a compound which:

(i) attenuates the increase in thioflavin T fluorescence by greater than 90%, relative to beta-amyloid with vehicle as a control, at 20 hours when subjected to a beta-amyloid thioflavin T aggregation assay, and

(ii) attenuates an increase in fluorescence by greater than 30%, greater than 60% or greater than 90% at 20 hours, relative to tau441 with vehicle as a control, in a tau thioflavin S aggregation assay.

In certain embodiments, the invention is directed to a method of treating a protein folding disorder comprising administering to a patient in need thereof an effective amount of a compound which:

(i) attenuates the increase in thioflavin T fluorescence by greater than 30%, relative to beta-amyloid with vehicle as a control, at 30 hours when subjected to a beta-amyloid thioflavin T aggregation assay, and

(ii) attenuates an increase in fluorescence by greater than 30%, greater than 60% or greater than 90% at 30 hours, relative to tau441 with vehicle as a control, in a tau thioflavin S aggregation assay.

In certain embodiments, the invention is directed to a method of treating a protein folding disorder comprising administering to a patient in need thereof an effective amount of a compound which:

(i) attenuates the increase in thioflavin T fluorescence by greater than 60%, relative to beta-amyloid with vehicle as a control, at 30 hours when subjected to a beta-amyloid thioflavin T aggregation assay, and

(ii) attenuates an increase in fluorescence by greater than 30%, greater than 60% or greater than 90% at 30 hours, relative to tau441 with vehicle as a control, in a tau thioflavin S aggregation assay.

In certain embodiments, the invention is directed to a method of treating a protein folding disorder comprising administering to a patient in need thereof an effective amount of a compound which:

(i) attenuates the increase in thioflavin T fluorescence by greater than 90%, relative to beta-amyloid with vehicle as a control, at 30 hours when subjected to a beta-amyloid thioflavin T aggregation assay, and

(ii) attenuates an increase in fluorescence by greater than 30%, greater than 60% or greater than 90% at 30 hours, relative to tau441 with vehicle as a control, in a tau thioflavin S aggregation assay.

In certain embodiments, the invention is directed to a compound which attenuates an increase in thioflavin S fluorescence by greater than 30% at 20 hours, relative to tau441 with vehicle as a control, in a tau aggregation assay.

In certain embodiments, the invention is directed to compound which attenuates an increase in thioflavin S fluorescence by greater than 60% at 20 hours, relative to tau441 with vehicle as a control, in a tau aggregation assay.

In certain embodiments, the invention is directed to a compound which attenuates an increase in thioflavin S fluorescence by greater than 90% at 20 hours, relative to tau441 with vehicle as a control, in a tau aggregation assay.

In certain embodiments, the invention is directed to a compound which attenuates an increase in thioflavin S fluorescence by greater than 30% at 30 hours, relative to tau441 with vehicle as a control, in a tau aggregation assay.

In certain embodiments, the invention is directed to a compound which attenuates an increase in thioflavin S fluorescence by greater than 60% at 30 hours, relative to tau441 with vehicle as a control, in a tau aggregation assay.

In certain embodiments, the invention is directed to a compound which attenuates an increase in thioflavin S fluorescence by greater than 90% at 30 hours, relative to tau441 with vehicle as a control, in a tau aggregation assay.

In certain embodiments, the invention is directed to a compound which:

(i) attenuates the increase in thioflavin T fluorescence by greater than 30%, relative to beta-amyloid with vehicle as a control, at 20 hours when subjected to a beta-amyloid thioflavin T aggregation assay, and

(ii) attenuates an increase in fluorescence by greater than 30%, greater than 60% or greater than 90% at 20 hours, relative to tau441 with vehicle as a control, in a tau thioflavin S aggregation assay.

In certain embodiments, the invention is directed to a compound which:

(i) attenuates the increase in thioflavin T fluorescence by greater than 60%, relative to beta-amyloid with vehicle as a control, at 20 hours when subjected to a beta-amyloid thioflavin T aggregation assay, and

(ii) attenuates an increase in fluorescence by greater than 30%, greater than 60% or greater than 90% at 20 hours, relative to tau441 with vehicle as a control, in a tau thioflavin S aggregation assay.

In certain embodiments, the invention is directed to a compound which:

(i) attenuates the increase in thioflavin T fluorescence by greater than 90%, relative to beta-amyloid with vehicle as a control, at 20 hours when subjected to a beta-amyloid thioflavin T aggregation assay, and

(ii) attenuates an increase in fluorescence by greater than 30%, greater than 60% or greater than 90% at 20 hours, relative to tau441 with vehicle as a control, in a tau thioflavin S aggregation assay.

In certain embodiments, the invention is directed to a compound which:

(i) attenuates the increase in thioflavin T fluorescence by greater than 30%, relative to beta-amyloid with vehicle as a control, at 30 hours when subjected to a beta-amyloid thioflavin T aggregation assay, and

(ii) attenuates an increase in fluorescence by greater than 30%, greater than 60% or greater than 90% at 30 hours, relative to tau441 with vehicle as a control, in a tau thioflavin S aggregation assay.

In certain embodiments, the invention is directed to a compound which:

(i) attenuates the increase in thioflavin T fluorescence by greater than 60%, relative to beta-amyloid with vehicle as a control, at 30 hours when subjected to a beta-amyloid thioflavin T aggregation assay, and

(ii) attenuates an increase in fluorescence by greater than 30%, greater than 60% or greater than 90% at 30 hours, relative to tau441 with vehicle as a control, in a tau thioflavin S aggregation assay.

In certain embodiments, the invention is directed to a compound which:

(i) attenuates the increase in thioflavin T fluorescence by greater than 90%, relative to beta-amyloid with vehicle as a control, at 30 hours when subjected to a beta-amyloid thioflavin T aggregation assay, and

(ii) attenuates an increase in fluorescence by greater than 30%, greater than 60% or greater than 90% at 30 hours, relative to tau441 with vehicle as a control, in a tau thioflavin S aggregation assay.

In certain embodiments disclosed herein, the aggregation assay utilizes the conditions set forth in FIG. 13 (A-D).

In certain embodiments, the invention is directed to a method for the treatment of a protein folding disorder in a subject comprising administering an effective amount of a therapeutic agent to said patient, wherein said therapeutic agent binds to at least one of a BXBB, BBXB, AXBBXB or BXBBXA receptor site of a protein associated with the protein folding disorder. The binding of therapeutic agents at the BXBB, BBXB, AXBBXB and BXBBXA receptor sites is described in FEBS Letters 2005, “The ‘promiscuous drug concept’ with applications to Alzheimer's disease”, Stephenson V C et al. 2005. FEBS Lett 579:1338-42, the disclosure of which is hereby incorporated by reference. The FEBS Letters discusses that, arguably, Alzheimer's disease (AD) is a multifactorial syndrome, rather than single disease, arising from a complex array of neurochemical factors. Numerous studies on the molecular pathogenesis of AD implicate a diversity of factors ranging from neurotoxic peptides (β-amyloid) to inflammatory processes (interleukins), but all culminating in a common neuropathology. This diversity of molecular causation is an impediment to the design of effective therapies for AD. To address this design problem, we sought to identify a single, common motif (a “common receptor”) shared by multiple structurally and functionally diverse proteins implicated in AD. This search revealed the presence of a common BBXB peptide motif and upon refinement, an AXBBXB motif; these regions can be exploited for the design of a “promiscuous drug” that exploits a “one-drug-multiple-receptors” therapeutic strategy for AD.

The novel concept of a promiscuous drug addresses the emerging need for one-drug-multiple-target therapeutics. A promiscuous drug candidate is not a collection of different drug molecules combined in a single pill to act on a multitude of receptors implicated in the pathogenesis of a single disease; rather, it is a single entity that occupies specific and discrete volumes of biological space common to multiple different receptor targets. Given its complex multifactorial etiology, it is highly probable that AD and other protein folding disorders may benefit from such a promiscuous drug strategy. The BBXB and AXBBXB motifs identified in this study represent targets worthy of promiscuous drug design.

In addition to providing an enabling strategy for promiscuous drug design, this unbiased computer-driven identification of the BBXB/AXBBXB motifs may also provide fundamental insights into the biochemical basis of AD. The effective binding of the glycosaminoglycan (GAG), heparin, to the identified domains in 27 Alzheimer's associated proteins also strengthens the design possibilities for promiscuous drug therapeutics, as well as providing a harmonized structural basis for understanding and combating the immunopathology of AD.

In certain embodiments, the therapeutic agent has a binding energy to the 1AMB PDB Structure of the HHQK region of Aβ greater than −54.4 kcal/mol at the His13-His14 region.

In certain embodiments, the therapeutic agent has a binding energy to the 1AMB PDB Structure of the HHQK region of Aβ greater than −60.0 kcal/mol at the His13-His14 region.

In certain embodiments, the therapeutic agent has a binding energy to the 1AMB PDB Structure of the HHQK region of Aβ greater than −65.0 kcal/mol at the His13-His14 region.

In certain embodiments, the therapeutic agent has a binding energy to the 1AMB PDB Structure of the HHQK region of Aβ greater than −70.0 kcal/mol at the His13-His14 region.

In certain embodiments, the therapeutic agent has a binding energy to the 1AMC PDB Structure of the HHQK region of Aβ greater than −44.6 kcal/mol at the His13-His14 region.

In certain embodiments, the therapeutic agent has a binding energy to the 1AMC PDB Structure of the HHQK region of Aβ greater than −50.0 kcal/mol at the His13-His14 region.

In certain embodiments, the therapeutic agent has a binding energy to the 1AMC PDB Structure of the HHQK region of Aβ greater than −55.0 kcal/mol at the His13-His14 region.

In certain embodiments, the therapeutic agent has a binding energy to the 1AMC PDB Structure of the HHQK region of Aβ greater than −60.0 kcal/mol at the His13-His14 region.

In certain embodiments, the therapeutic agent has a binding energy to the 1AML PDB Structure of the HHQK region of Aβ greater than −35.6 kcal/mol at the His13-His14 region.

In certain embodiments, the therapeutic agent has a binding energy to the 1AML PDB Structure of the HHQK region of Aβ greater than −40.0 kcal/mol at the His13-His14 region.

In certain embodiments, the therapeutic agent has a binding energy to the 1AML PDB Structure of the HHQK region of Aβ greater than −45.0 kcal/mol at the His13-His14 region.

In certain embodiments, the therapeutic agent has a binding energy to the 1AML PDB Structure of the HHQK region of Aβ greater than −50.0 kcal/mol at the His13-His14 region.

In certain embodiments, the therapeutic agent has a binding energy to the 1BA4 PDB Structure of the HHQK region of Aβ greater than −27.1 kcal/mol at the His13-His14 region.

In certain embodiments, the therapeutic agent has a binding energy to the 1BA4 PDB Structure of the HHQK region of Aβ greater than −30.0 kcal/mol at the His13-His14 region.

In certain embodiments, the therapeutic agent has a binding energy to the 1BA4 PDB Structure of the HHQK region of Aβ greater than −35.0 kcal/mol at the His 13-His14 region.

In certain embodiments, the therapeutic agent has a binding energy to the 1BA4 PDB Structure of the HHQK region of Aβ greater than −40.0 kcal/mol at the His13-His14 region.

In certain embodiments, the therapeutic agent has a binding energy to the 1IYT PDB Structure of the HHQK region of Aβ greater than −36.8 kcal/mol at the His13-His14 region.

In certain embodiments, the therapeutic agent has a binding energy to the 1IYT PDB Structure of the HHQK region of Aβ greater than −40.0 kcal/mol at the His13-His14 region.

In certain embodiments, the therapeutic agent has a binding energy to the 1IYT PDB Structure of the HHQK region of Aβ greater than −45.0 kcal/mol at the His13-His14 region.

In certain embodiments, the therapeutic agent has a binding energy to the 1IYT PDB Structure of the HHQK region of Aβ greater than −50.0 kcal/mol at the His13-His14 region.

In certain embodiments, the therapeutic agent has a binding energy to the 2BP4 PDB Structure of the HHQK region of Aβ greater than −32.7 kcal/mol at the His13-His14 region.

In certain embodiments, the therapeutic agent has a binding energy to the 2BP4 PDB Structure of the HHQK region of Aβ greater than −35.0 kcal/mol at the His13-His14 region.

In certain embodiments, the therapeutic agent has a binding energy to the 2BP4 PDB Structure of the HHQK region of Aβ greater than −40.0 kcal/mol at the His13-His14 region.

In certain embodiments, the therapeutic agent has a binding energy to the 2BP4 PDB Structure of the HHQK region of Aβ greater than −40.0 kcal/mol at the His13-His14 region.

In certain embodiments, the therapeutic agent has a binding energy to the 1AMB PDB Structure of the HHQK region of Aβ greater than −43.5 kcal/mol at the His13-Lys16 region.

In certain embodiments, the therapeutic agent has a binding energy to the 1AMB PDB Structure of the HHQK region of Aβ greater than −45.0 kcal/mol at the His13-Lys16 region.

In certain embodiments, the therapeutic agent has a binding energy to the 1AMB PDB Structure of the HHQK region of Aβ greater than −50.0 kcal/mol at the His13-Lys16 region.

In certain embodiments, the therapeutic agent has a binding energy to the 1AMB PDB Structure of the HHQK region of Aβ greater than −55.0 kcal/mol at the His13-Lys16 region.

In certain embodiments, the therapeutic agent has a binding energy to the 1AMC PDB Structure of the HHQK region of Aβ greater than −34.3 kcal/mol at the His13-Lys16 region.

In certain embodiments, the therapeutic agent has a binding energy to the 1AMC PDB Structure of the HHQK region of Aβ greater than −40.0 kcal/mol at the His13-Lys16 region.

In certain embodiments, the therapeutic agent has a binding energy to the 1AMC PDB Structure of the HHQK region of Aβ greater than −45.0 kcal/mol at the His13-Lys16 region.

In certain embodiments, the therapeutic agent has a binding energy to the 1AMC PDB Structure of the HHQK region of Aβ greater than −50.0 kcal/mol at the His13-Lys16 region.

In certain embodiments, the therapeutic agent has a binding energy to the 1AML PDB Structure of the HHQK region of Aβ greater than −22.3 kcal/mol at the His13-Lys16 region.

In certain embodiments, the therapeutic agent has a binding energy to the 1AML PDB Structure of the HHQK region of Aβ greater than −25.0 kcal/mol at the His13-Lys16 region.

In certain embodiments, the therapeutic agent has a binding energy to the 1AML PDB Structure of the HHQK region of Aβ greater than −30.0 kcal/mol at the His13-Lys16 region.

In certain embodiments, the therapeutic agent has a binding energy to the 1AML PDB Structure of the HHQK region of Aβ greater than −35.0 kcal/mol at the His13-Lys16 region.

In certain embodiments, the therapeutic agent has a binding energy to the 1BA4 PDB Structure of the HHQK region of Aβ greater than −46.0 kcal/mol at the His 13-Lys16 region.

In certain embodiments, the therapeutic agent has a binding energy to the 1BA4 PDB Structure of the HHQK region of Aβ greater than −50.0 kcal/mol at the His13-Lys16 region.

In certain embodiments, the therapeutic agent has a binding energy to the 1BA4 PDB Structure of the HHQK region of Aβ greater than −55.0 kcal/mol at the His13-Lys16 region.

In certain embodiments, the therapeutic agent has a binding energy to the 1BA4 PDB Structure of the HHQK region of Aβ greater than −60.0 kcal/mol at the His13-Lys16 region.

In certain embodiments, the therapeutic agent has a binding energy to the 1IYT PDB Structure of the HHQK region of Aβ greater than −17.3 kcal/mol at the His13-Lys16 region.

In certain embodiments, the therapeutic agent has a binding energy to the 1IYT PDB Structure of the HHQK region of Aβ greater than −20.0 kcal/mol at the His13-Lys16 region.

In certain embodiments, the therapeutic agent has a binding energy to the 1IYT PDB Structure of the HHQK region of Aβ greater than −25.0 kcal/mol at the His13-Lys16 region.

In certain embodiments, the therapeutic agent has a binding energy to the 1IYT PDB Structure of the HHQK region of Aβ greater than −30.0 kcal/mol at the His13-Lys16 region.

In certain embodiments, the therapeutic agent has a binding energy to the 2BP4 PDB Structure of the HHQK region of Aβ greater than −48.6 kcal/mol at the His13-Lys16 region.

In certain embodiments, the therapeutic agent has a binding energy to the 2BP4 PDB Structure of the HHQK region of Aβ greater than −50.0 kcal/mol at the His13-Lys16 region.

In certain embodiments, the therapeutic agent has a binding energy to the 2BP4 PDB Structure of the HHQK region of Aβ greater than −55.0 kcal/mol at the His13-Lys16 region.

In certain embodiments, the therapeutic agent has a binding energy to the 2BP4 PDB Structure of the HHQK region of Aβ greater than −60.0 kcal/mol at the His13-Lys16 region.

For purposes of this invention, the term “binding energy” means the energy required to separate particles from a molecule or atom or nucleus. Therefore, the more negative the number is, the more energy is required for separation, thus a greater binding energy, and thus greater binding affinity.

In certain embodiments, the HHQK region includes the portions described above and in Table 18A of Example 18.

In certain embodiments, the therapeutic agent has a binding energy to the HHQK region of Aβ that is 2% greater than the binding energy of L-tryptophan to the HHQK region of Aβ.

In certain embodiments, the therapeutic agent has a binding energy to the HHQK region of Aβ that is 5% greater than the binding energy of L-tryptophan to the HHQK region of Aβ.

In certain embodiments, the therapeutic agent has a binding energy to the HHQK region of Aβ that is 10% greater than the binding energy of L-tryptophan to the HHQK region of Aβ.

In certain embodiments, the binding energy is measured using the CHARMM27 force field and explicit solvation.

In other embodiments of the invention, the therapeutic agent is a compound as disclosed herein. In other embodiments, the therapeutic agent is not L-tryptophan. In other embodiments, the therapeutic agent is not a tryptophan dipeptide.

In certain embodiments of the invention, the protein folding disorder is Alzheimer's Disease.

In certain embodiments of the invention, the therapeutic agent has a bonding distance to the BXBB, BBXB, AXBBXB or BXBBXA receptor site of from about 1.63 to about 3.48 Å.

In certain embodiments of the disclosed method, the protein folding disorder being treated is a neurodegenerative disease.

In certain embodiments of the disclosed method, the neurodegenerative disease is selected from the group consisting of tauopathies, cerebral amyloid angiopathy, Lewy body diseases (e.g. Parkinson's disease), Alzheimer's disease, dementia, Huntington's disease, prion-based spongiform encephalopathy and a combination thereof.

In certain embodiments of the disclosed method, the neurodegenerative disease is Alzheimer's disease.

In certain embodiments, the present invention is directed to a pharmaceutical composition comprising a pharmaceutically acceptable excipient and an effective amount of a compound of formula (I), (II), (III), (IV), (V), (VI), (VII) or (VIII) to treat a protein folding disorder, e.g., a neurodegenerative disease such as, tauopathies, cerebral amyloid angiopathy, Lewy body diseases (e.g. Parkinson's disease), Alzheimer's disease, dementia, Huntington's disease, prion-based spongiform encephalopathy and a combination thereof.

In certain embodiments, the present invention is directed to a pharmaceutical composition comprising a pharmaceutically acceptable excipient and an effective amount of a compound of formula (I), (II), (III), (IV), (V), (VI), (VII) or (VIII) to treat systemic amyloidoses, particularly those affecting the peripheral nerves, spleen and pancreas.

In certain embodiments, the invention is directed to a method for treating a protein folding disorder comprising administering a compound or pharmaceutical composition as disclosed herein to a subject wherein the subject is treated for the protein folding disorder.

In certain embodiments, the invention is directed to a method for treating a protein folding disorder comprising administering an effective amount of a compound or pharmaceutical composition as disclosed herein to a patient in need thereof.

In certain embodiments, the compounds of the present invention are non-peptides.

As used herein, the term “alkyl” means a linear or branched saturated aliphatic hydrocarbon group having a single radical and 1-10 carbon atoms. Examples of alkyl groups include methyl, propyl, isopropyl, butyl, n-butyl, isobutyl, sec-butyl, tert-butyl, and pentyl. A branched alkyl means that one or more alkyl groups such as, e.g., methyl, ethyl or propyl, replace one or both hydrogens in a —CH₂— group of a linear alkyl chain. The term “lower alkyl” means an alkyl of 1-3 carbon atoms.

The term “alkoxy” means an “alkyl” as defined above connected to an oxygen radical.

The term “cycloalkyl” means a non-aromatic mono- or multicyclic hydrocarbon ring system having a single radical and 3-12 carbon atoms. Exemplary monocyclic cycloalkyl rings include cyclopropyl, cyclopentyl, and cyclohexyl. Exemplary multicyclic cycloalkyl rings include adamantyl and norbornyl.

The term “alkenyl” means a linear or branched aliphatic hydrocarbon group containing a carbon-carbon double bond having a single radical and 2-10 carbon atoms.

A “branched” alkenyl means that one or more alkyl groups such as, e.g., methyl, ethyl or propyl replace one or both hydrogens in a —CH₂— or —CH═ linear alkenyl chain. Exemplary alkenyl groups include ethenyl, 1- and 2-propenyl, 1-, 2- and 3-butenyl, 3-methylbut-2-enyl, heptenyl, octenyl and decenyl.

The term “cycloalkenyl” means a non-aromatic monocyclic or multicyclic hydrocarbon ring system containing a carbon-carbon double bond having a single radical and 3 to 12 carbon atoms. Exemplary monocyclic cycloalkenyl rings include cyclopropenyl, cyclopentenyl, cyclohexenyl or cycloheptenyl. An exemplary multicyclic cycloalkenyl ring is norbornenyl.

The term “alkynyl” means a linear or branched aliphatic hydrocarbon group containing a carbon-carbon triple bond having a single radical and 2-10 carbon atoms.

A “branched” alkynyl means that one or more alkyl groups such as, e.g., methyl, ethyl or propyl replace one or both hydrogens in a —CH₂— linear alkynyl chain.

The term “cycloalkynyl” means a non-aromatic monocyclic or multicyclic hydrocarbon ring system containing a carbon-carbon triple bond having a single radical and 3 to 12 carbon atoms.

The term “aryl” means a carbocyclic aromatic ring system containing one, two or three rings which may be attached together in a pendent manner or fused, and containing a single radical. Exemplary aryl groups include phenyl, naphthyl and acenaphthyl.

The term “heteroaryl” means unsaturated heterocyclic radicals. Exemplary heteroaryl groups include unsaturated 3 to 6 membered hetero-monocyclic groups containing 1 to 4 nitrogen atoms, such as, e.g., pyrrolyl, pyridyl, pyrimidyl, and pyrazinyl; unsaturated condensed heterocyclic groups containing 1 to 5 nitrogen atoms, such as, e.g., indolyl, quinolyl and isoquinolyl; unsaturated 3 to 6-membered hetero-monocyclic groups containing an oxygen atom, such as, e.g., furyl; unsaturated 3 to 6 membered hetero-monocyclic groups containing a sulfur atom, such as, e.g., thienyl; unsaturated 3 to 6 membered hetero-monocyclic groups containing 1 to 2 oxygen atoms and 1 to 3 nitrogen atoms, such as, e.g., oxazolyl; unsaturated condensed heterocyclic groups containing 1 to 2 oxygen atoms and 1 to 3 nitrogen atoms, such as, e.g., benzoxazolyl; unsaturated 3 to 6 membered hetero-monocyclic groups containing 1 to 2 sulfur atoms and 1 to 3 nitrogen atoms, such as, e.g., thiazolyl; and unsaturated condensed heterocyclic group containing 1 to 2 sulfur atoms and 1 to 3 nitrogen atoms, such as, e.g., benzothiazolyl. The term “heteroaryl” also includes unsaturated heterocyclic radicals, wherein “heterocyclic” is as previously described, in which the heterocyclic group is fused with an aryl group, in which aryl is as previously described. Exemplary fused radicals include benzofuran, benzodioxole and benzothiophene.

The term “carbonyl”, whether used alone or with other terms, such as, e.g., “alkoxycarbonyl”, is (C═O).

The term “alkylcarbonyl” includes radicals having alkyl radicals, as defined above, attached to a carbonyl radical.

The term “carboxylic acid” is CO₂H.

All of the cyclic ring structures disclosed herein can be attached at any point where such connection is possible, as recognized by one skilled in the art.

The terms “bi-indole” and “bis-indole” are used interchangeably.

As used herein, the term “subject” includes a human or an animal such as, e.g., a companion animal or livestock.

The term “patient” includes a subject in need of therapeutic treatment.

As used herein, the term “halogen” or “halo” includes fluoride, bromide, chloride, iodide or astatide.

For purposes of the present invention the abbreviation “Trp” means tryptophan.

There are many different isoforms of tau which can be utilized in a tau aggregation assay. The particular tau isoform utilized in the present invention is not meant to limit the scope of the invention which encompasses tau aggregation assays utilizing any suitable tau isomer.

For purposes of the present invention, wherein the formula includes a q₁, q₁ and/or a q₂ variable, when the q, q₁ and/or a q₂ variable is less than 4, it is understood that the base structure will include hydrogen substituents where necessary (e.g., on the aromatic ring) to complete valence.

The invention disclosed herein is meant to encompass all pharmaceutically acceptable salts thereof of the disclosed compounds. The pharmaceutically acceptable salts include, but are not limited to, metal salts such as, e.g., sodium salt, potassium salt, cesium salt and the like; alkaline earth metals such as, e.g., calcium salt, magnesium salt and the like; organic amine salts such as, e.g., triethylamine salt, pyridine salt, picoline salt, ethanolamine salt, triethanolamine salt, dicyclohexylamine salt, N,N′-dibenzylethylenediamine salt and the like; inorganic acid salts such as, e.g., hydrochloride, hydrobromide, sulfate, phosphate and the like; organic acid salts such as, e.g., formate, acetate, trifluoroacetate, maleate, fumarate, tartrate and the like; sulfonates such as, e.g., methanesulfonate, benzenesulfonate, p-toluenesulfonate, and the like; amino acid salts such as, e.g., arginate, asparginate, glutamate and the like.

The invention disclosed herein is also meant to encompass all prodrugs of the disclosed compounds. Prodrugs are considered to be any covalently bonded carriers which release the active parent drug in vivo. An example of a prodrug would be an ester which is processed in vivo to a carboxylic acid or salt thereof.

The invention disclosed herein is also meant to encompass the in vivo metabolic products of the disclosed compounds. Such products may result for example from the oxidation, reduction, hydrolysis, amidation, esterification and the like of the administered compound, primarily due to enzymatic processes. Accordingly, the invention includes compounds produced by a process comprising contacting a compound of this invention with a mammal for a period of time sufficient to yield a metabolic product thereof. Such products typically are identified by preparing a radiolabelled compound of the invention, administering it parenterally in a detectable dose to an animal such as, e.g., a rat, mouse, guinea pig, monkey, or to man, allowing sufficient time for metabolism to occur and isolating its conversion products from the urine, blood or other biological samples. One skilled in the art recognizes that interspecies pharmacokinetic scaling can be used to study the underlining similarities (and differences) in drug disposition among species, to predict drug disposition in an untested species, to define pharmacokinetic equivalence in various species, and to design dosage regimens for experimental animal models, as discussed in Mordenti, Man versus Beast: Pharmacokinetic Scaling in Mammals, 1028, Journal of Pharmaceutical Sciences, Vol. 75, No. 11, November 1986.

The invention disclosed herein is also meant to encompass the disclosed compounds being isotopically-labelled by having one or more atoms replaced by an atom having a different atomic mass or mass number. Examples of isotopes that can be incorporated into the disclosed compounds include isotopes of hydrogen, carbon, nitrogen, oxygen, phosphorous, fluorine and chlorine, such as, e.g., ²H, ³H, ¹³C, ¹⁴C, ¹⁵N, ¹⁸O, ¹⁷O, ³¹P, ³²P, ³⁵S, ¹⁸F, and ³⁶Cl, respectively. Some of the compounds disclosed herein may contain one or more asymmetric centers and may thus give rise to enantiomers, diastereomers, and other stereoisomeric forms. The present invention is also meant to encompass all such possible forms as well as their racemic and resolved forms and mixtures thereof. When the compounds described herein contain olefinic double bonds or other centers of geometric asymmetry, and unless specified otherwise, it is intended to include both E and Z geometric isomers. All tautomers are intended to be encompassed by the present invention as well.

As used herein, the term “stereoisomers” is a general term for all isomers of individual molecules that differ only in the orientation of their atoms in space. It includes enantiomers and isomers of compounds with more than one chiral center that are not mirror images of one another (diastereomers).

The term “chiral center” refers to a carbon atom to which four different groups are attached.

The term “enantiomer” or “enantiomeric” refers to a molecule that is nonsuperimposeable on its mirror image and hence optically active wherein the enantiomer rotates the plane of polarized light in one direction and its mirror image rotates the plane of polarized light in the opposite direction.

The term “racemic” refers to a mixture of equal parts of enantiomers and which is optically inactive.

The term “resolution” refers to the separation or concentration or depletion of one of the two enantiomeric forms of a molecule.

### BRIEF DESCRIPTION OF THE DRAWINGS

FIG. 1 (A-G) depicts the inhibition of Aβ¹⁻⁴⁰ aggregation bycompounds of the present invention as shown in Example 8, as measured by Thioflavin T (ThT) fluorescence.

FIG. 2 depicts the dose-response effect of a compound of the present invention on Aβ¹⁻⁴⁰ aggegation in kinetic ThT assay as shown in Example 8.

FIG. 3 depicts dose-response curves for inhibition of Aβ¹⁻⁴⁰ aggregation by a compound of the present invention and a control as shown in Example 8.

FIG. 4 depicts the inhibition of Aβ¹⁻⁴⁰ aggregation by a compound of the present invention as shown in Example 8 in a “seeded” ThT assay.

FIG. 5 depicts disaggregation of Aβ¹⁻⁴⁰ by a compound of the present invention and a control in a ThT assay as shown in Example 8.

FIGS. 6A-C depict circular dichroism studies of compounds of the present invention and a control as shown in Example 8.

FIG. 7A-C depicts the inhibition of Aβ¹⁻⁴² aggregation by compounds of the current invention as shown in Example 8, as measured by ThT fluorescence.

FIG. 8 depicts the dose-response curves for inhibition of Aβ¹⁻⁴² aggregation by compounds of the current invention as shown in Example 8, as measured by ThT fluorescence.

FIG. 9 depicts an ¹H NMR binding study for a compound of the current invention to Aβ¹⁻⁴⁰, as shown in Example 8.

FIG. 10A-E depicts the inhibition, or lack thereof, of tau aggregation, seen as both a reduced rate of tau aggregation and reduced equilibrium or plateau level of aggregation, by compounds of the current invention and nicotinic acid, as shown in Example 8, as measured by Thioflavin S (ThS) fluorescence. The inhibition of tau aggregation is by synthesized bi-aromatic compounds and morin.

FIG. 11A-D depicts the effect on tau aggregation of synthesized bi-aromatic compounds and nicotinic acid and the modulation, or lack thereof, of tau aggregation, seen as an increased initial rate of tau aggregation but reduced equilibrium or plateau level of aggregation, by compounds of the current invention, as shown in Example 8, as measured by ThS fluorescence.

FIG. 12A-B depicts inhibition of α-synuclein aggregation by compounds of the present invention as shown in Example 8, as measured by ThT fluorescence.

FIG. 13 depicts the mean (±SE) change in primary efficacy variables from baseline in a human clinical trial of L-Trp in people with AD; * p<0.001, † p<0.01, from Example 16.

FIG. 14 depicts “typical” binding of L-Trp to HHQK region of Aβ wherein it is shown binding to His₁₃ and Lys₁₆ of PDB structure 1AML as discussed in Example 17.

FIG. 15 depicts alternative binding of L-Trp to HHQK region of Aβ wherein it is shown occurring to His₁₄ and Lys₁₆ of PDB structure 1BA4 as discussed in Example 17.

FIG. 16 depicts the interaction of 0c with KREH receptor of B7-1 as discussed in Example 18.

FIG. 17 depicts the interaction of 0c with RDHH receptor of ICAM-1 as discussed in Example 18.

FIG. 18 depicts the interaction of 0c with HKEK receptor of IL-1R1 as discussed in Example 18.

### DETAILED DESCRIPTION

The compounds of the present invention can be administered to anyone requiring treatment of a protein folding disease or systemic amyloidoses. For example, the compounds are useful for treating Alzheimer's disease, for helping prevent or delay the onset of Alzheimer's disease, for treating patients with MCI (mild cognitive impairment) and preventing or delaying the onset of Alzheimer's disease in those who would progress from MCI to AD, for treating Down's syndrome, for treating humans who have Hereditary Cerebral Hemorrhage with Amyloidosis of the Dutch-Type, for treating cerebral amyloid angiopathy and preventing its potential consequences, i.e. single and recurrent lobal hemorrhages, for treating other degenerative dementias, including dementias of mixed vascular and degenerative origin, dementia associated with Parkinson's disease, dementia associated with progressive supranuclear palsy, dementia associated with cortical basal degeneration, dementia associated with tauopathies, and diffuse Lewy body type Alzheimer's disease. Preferably, the compounds and compositions of the invention are particularly useful for treating or preventing Alzheimer's disease.

Di- and polyanionic sulfate and sulfonate compounds have been shown to inhibit in vitro aggregation of amyloidogenic proteins, including the Alzheimer peptide, Aβ (Kisilevsky et al., Nat. Med., 1:143-8, 1995). It is thought that these anionic compounds in vivo would inhibit Aβ deposition by disrupting Aβ-glycosaminoglycan.

It is believed that the compounds and methods of the present invention will result in a therapeutic outcome by binding the His₁₃-His₁₄-Gln₁₅-Lys₁₆ region of Aβ via cation-π interactions, rather than cationic-anionic interactions. Without being bound by theory, it is believed that the compounds of the present invention containing two aromatic groups would form cation-π interactions at two of the three cationic residues in the His₁₃-His₁₄-Gln₁₅-Lys₁₆ region and thereby interfere with Aβ aggregation (See, The HHQK Domain of β-Amyloid Provides a Structural Basis for the Immunopathology of Alzeheimer's Disease, The Journal of Biological Chemistry, Vol. 274, No. 45, pp 29719-29726, 1988).

The compounds of certain embodiments of the invention are non-peptidic, small organic molecules. Because of this, they are expected to overcome deficiencies of peptidic compounds such as poor pharmacokinetics, e.g., degradation by proteases.

When treating or preventing these diseases, the compounds of the invention can either be used individually or in combination. For example, aministration may be orally, topically, by suppository, inhalation, subcutaneously, intravenously, bucally, sublingually, or parenterally.

Various oral dosage forms can be used, including such solid forms as tablets, gelcaps, capsules, caplets, granules, lozenges and bulk powders and liquid forms such as, e.g., emulsions, solution and suspensions. The compounds of the present invention can be administered alone or can be combined with various pharmaceutically acceptable carriers and excipients known to those skilled in the art, including but not limited to diluents, suspending agents, solubilizers, binders, disintegrants, preservatives, coloring agents, lubricants and the like.

When the compounds of the present invention are incorporated into oral tablets, such tablets can be compressed, tablet triturates, enteric-coated, sugar-coated, film-coated, multiply compressed or multiply layered. Liquid oral dosage forms include aqueous and nonaqueous solutions, emulsions, suspensions, and solutions and/or suspensions reconstituted from non-effervescent granules, containing suitable solvents, preservatives, emulsifying agents, suspending agents, diluents, sweeteners, coloring agents, and flavoring agents. When the compounds of the present invention are to be injected parenterally, they may be, e.g., in the form of an isotonic sterile solution. Alternatively, when the compounds of the present invention are to be inhaled, they may be formulated into a dry aerosol or may be formulated into an aqueous or partially aqueous solution.

In addition, when the compounds of the present invention are incorporated into oral dosage forms, it is contemplated that such dosage forms may provide an immediate release of the compound in the gastrointestinal tract, or alternatively may provide a controlled and/or sustained release through the gastrointestinal tract. A wide variety of controlled and/or sustained release formulations are well known to those skilled in the art, and are contemplated for use in connection with the formulations of the present invention. The controlled and/or sustained release may be provided by, e.g., a coating on the oral dosage form or by incorporating the compound(s) of the invention into a controlled and/or sustained release matrix.

Specific examples of pharmaceutically acceptable carriers and excipients that may be used to formulate oral dosage forms, are described in the Handbook of Pharmaceutical Excipients, American Pharmaceutical Association (1986). Techniques and compositions for making solid oral dosage forms are described in Pharmaceutical Dosage Forms: Tablets (Lieberman, Lachman and Schwartz, editors) 2nd edition, published by Marcel Dekker, Inc. Techniques and compositions for making tablets (compressed and molded), capsules (hard and soft gelatin) and pills are also described in Remington's Pharmaceutical Sciences (Arthur Osol, editor), 1553B1593 (1980). Techniques and composition for making liquid oral dosage forms are described in Pharmaceutical Dosage Forms: Disperse Systems, (Lieberman, Rieger and Banker, editors) published by Marcel Dekker, Inc.

When the compounds of the present invention are incorporated for parenteral administration by injection (e.g., continuous infusion or bolus injection), the formulation for parenteral administration may be in the form of suspensions, solutions, emulsions in oily or aqueous vehicles, and such formulations may further comprise pharmaceutically necessary additives such as, e.g., stabilizing agents, suspending agents, dispersing agents, and the like. The compounds of the invention may also be in the form of a powder for reconstitution as an injectable formulation.

The compounds and compositions of the invention can be enclosed in multiple or single dose containers. The enclosed compounds and compositions can be provided in kits, for example, including component parts that can be assembled for use. The kit can also optionally include instructions for use in any medium. For example, the instructions can be in paper or electronic form. For example, a compound of the present invention in lyophilized form and a suitable diluent may be provided as separated components for combination prior to use. A kit may include a compound of the present invention and a second therapeutic agent for co-administration. The compound of the present invention and second therapeutic agent may be provided as separate component parts. A kit may include a plurality of containers, each container holding one or more unit dose of the compound of the invention. The containers are preferably adapted for the desired mode of administration, including, but not limited to tablets, gel capsules, sustained-release capsules, and the like for oral administration; depot products, pre-filled syringes, ampules, vials, and the like for parenternal administration; and patches, medipads, creams, and the like for topical administration.

The concentration of active compound in the drug composition will depend on absorption, inactivation, and excretion rates of the active compound, the dosage schedule, and amount administered as well as other factors known to those of skill in the art.

The active ingredient may be administered at once, or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

The compounds of the invention can be used in combination, with each other or with other therapeutic agents or approaches used to treat or prevent the protein folding conditions described above. Such agents include, for example, cholinesterase inhibitors (such as, e.g., acetylcholinesterase inhibitors and butyrylcholinesterase inhibitors); gamma-secretase inhibitors; beta-secretase inhibitors; anti-inflammatory agents; anti-oxidants; immunological approaches; NMDA antagonists; cholesterol lowering agents (such as, e.g., statins); and direct or indirect neurotropic agents.

Acetylcholinesterase inhibitors include compounds such as, e.g., tacrine (tetrahydroaminoacridine, marketed as Cognex®), donepezil hydrochloride, (marketed as Aricept®), rivastigmine (marketed as Exelon®) and galantamine (Reminyl®).

Anti-oxidants include compounds such as, e.g., tocopherol, ascorbic acid, beta carotene, lipoic acid, selenium, glutathione, cysteine, coenzyme Q, vitamin E and ginkolides.

NMDA (N-methyl-D-aspartate) antagonists include, for example, memantine (Namenda®).

Immunological approaches include, for example, immunization with beta-amyloid peptides (or fragments thereof) or administration of anti-beta-amyloid antibodies.

Direct or indirect neurotropics agents include, for example, Cerebrolysin® and AIT-082 (Emilieu, 2000, Arch. Neurol. 57:454).

Anti-inflammatory agents include, for example, Cox-II inhibitors such as, e.g., rofecoxib, celecoxib, DUP-697, flosulide, meloxicam, 6-MNA, L-745337, nabumetone, nimesulide, NS-398, SC-5766, T-614, L-768277, GR-253035, JTE-522, RS-57067-000, SC-58125, SC-078, PD-138387, NS-398, flosulide, D-1367, SC-5766, PD-164387, etoricoxib, valdecoxib, parecoxib and pharmaceutically acceptable salts thereof. Other anti-inflammatory agents include, for example, aspirin, ibuprofen, diclofenac, naproxen, benoxaprofen, flurbiprofen, fenoprofen, flubufen, ketoprofen, indoprofen, piroprofen, carprofen, oxaprozin, pramoprofen, muroprofen, trioxaprofen, suprofen, aminoprofen, tiaprofenic acid, fluprofen, bucloxic acid, indomethacin, sulindac, tolmetin, zomepirac, tiopinac, zidometacin, acemetacin, fentiazac, clidanac, oxpinac, mefenamic acid, meclofenamic acid, flufenamic acid, niflumic acid tolfenamic acid, diflurisal, flufenisal, piroxicam, sudoxicam, isoxicam and pharmaceutically acceptable salts thereof.

Statins include, for example, atorvastatin, simvastatin, pravastatin, cerivastatin, mevastatin, velostatin, fluvastatin, lovastatin, dalvastatin, rosuvastatin, fluindostatin, dalvastain and pharmaceutically acceptable salts thereof.

Other cholesterol reducing compounds include bile sequestration compounds (e.g., colestipol and cholestyramine); fibrin (e.g., gemfibrozil, fenofibrate, psyllium, wheat bran, oat bran, rice bran, corn bran, konjak flour, Jerusalem artichoke flour, fruit fiber and any other functional food products) and other agents such as, e.g., nicotinic acid (niacin).

In addition, the compounds of the invention can also be used with inhibitors of P-glycoprotein (P-gp). The use of P-gp inhibitors is known to those skilled in the art. See for example, Cancer Research, 53, 4595-4602 (1993), Clin. Cancer Res., 2, 7-12 (1996), Cancer Research, 56, 4171-4179 (1996), International Publications WO99/64001 and WO01/10387. P-gp inhibitors are useful by inhibiting P-gp from decreasing brain blood levels of the compounds of the invention. Suitable P-gp inhibitors include cyclosporin A, verapamil, tamoxifen, quinidine, Vitamin E-TGPS, ritonavir, megestrol acetate, progesterone, rapamycin, 10,11-methanodibenzosuberane, phenothiazines, acridine derivatives such as, e.g., GF120918, FK506, VX-710, LY335979, PSC-833, GF-102,918 and other steroids.

All of the additional agents disclosed above may be administered at the same or different time and/or route of administration than the compounds of the present invention.

The following examples illustrate various aspects of the present invention, and are not to be construed to limit the claims in any manner whatsoever.

### DETAILED DESCRIPTION OF PREFERRED EMBODIMENTS

The following examples refer to compounds listed in Table I below:

TABLE 1

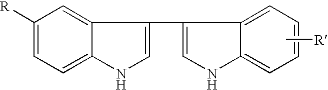

| | R | R′ |
|---|---|---|
| 0a | H | H |
| 0b | OCH₃ | 5-OCH₃ |
| 0c | Br | 5-CO₂H |
| 0d | H | 5-CO₂H |
| 0e | OCH₃ | 5-CO₂H |
| 0f | F | 5-CO₂H |
| 0g | Cl | 5-CO₂H |
| 0h | CH₃ | 5-CO₂H |
| 0i | CF₃ | 5-CO₂H |
| 0j | OH | 5-CO₂H |
| 0k | H | 6-CO₂H |
| 0l | H | 7-CO₂H |
| 0m | OH | 5-OH |

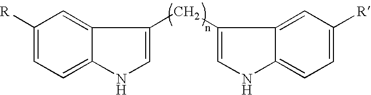

| | n | R | R′ |
|---|---|---|---|
| 1a | 1 | H | H |
| 1c | 1 | OCH₃ | OCH₃ |
| 1e | 1 | H | CO₂H |
| 1f | 1 | CO₂H | CO₂H |
| 1g | 1 | OH | OH |
| 2a | 2 | H | H |
| 2b | 2 | Br | CO₂H |
| 2c | 2 | CO₂H | CO₂H |
| 3 | 3 | H | H |
| 4 | 4 | H | H |
| 5 | 5 | H | H |
| 6 | 10 | H | H |
| 7 | p-xylyl | H | H |

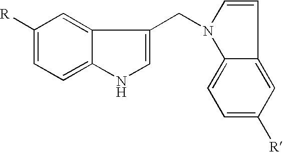

| | R | R′ |
|---|---|---|
| lb | H | H |
| Id | OCH₃ | OCH₃ |

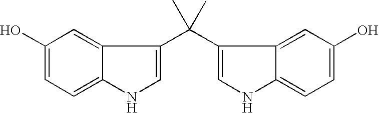

1h

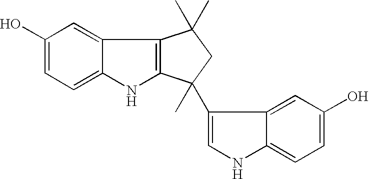

1i

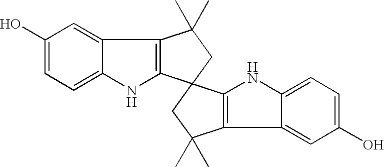

1j

### EXAMPLE 1

### Synthesis of Directly Linked Bis-Indoles

Directly linked bis-indoles were synthesized according to the procedure set forth in Scheme 1 below:

Scheme 1, Synthesis of 3,3′-bis-indolyl compounds

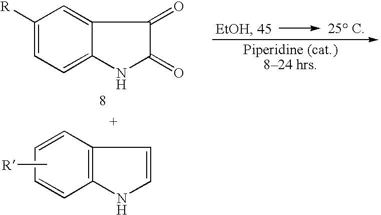

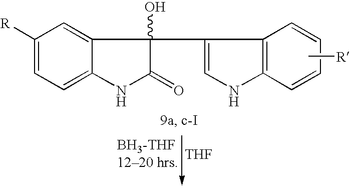

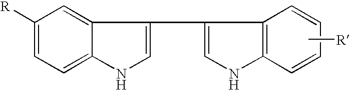

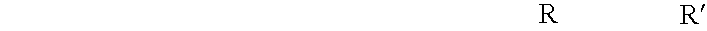

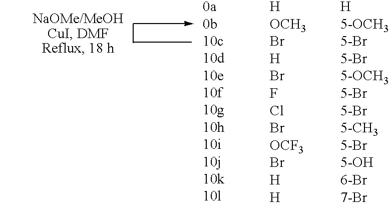

### (i) Coupling of Indole and Isatin (9a, c-1)

Following the procedure of Bergman (J. Acta. Chem. Scand., 1971, 4: 1277-80) a solution of isatin (5-20 mmol) and indole (1 eq.), either or both substituted at the 5, 6 or 7 position as appropriate (Scheme 1), and piperidine (0.1 eq.) were stirred in ethanol at 45° C. for one hour and then at room temperature. When TLC indicated the reaction was complete (8-24 hrs.), the reaction mixture was filtered in cases where a small amount of solid was present, and the product purified by recrystallization from EtOH/H₂O (9a,d,f,j), THF/EtOH/H₂O (9g,h), or EtOAc/hexanes (9i). For 9c and 9e, purification was achieved by removing the solvent from the reaction mixture, resuspending the yellow solid in EtOH (50 mL), sonicating for 2 hrs. to dissolve impurities, collecting the solid by vacuum filtration, and rinsing with EtOH.

All NMR chemical shift data presented herein is in ppm (parts per million) and J coupling contants are presented herein in Hertz. Due to symmetry, some ¹³C carbon peaks correspond to 2 carbons, where indicated by (2c).

### 3-Hydroxy-3-(indol-3-yl)-indolin-2-one (9a)

Light beige crystals (7.47 g, 94%); mp 120° C. (dec.), lit. 123° C. (dec.) (Berens U et al. 1996. Tetrahedron Asymmetry 7:285-92); ¹H NMR: 6.33 (s, 1H), 6.88 (m, 2H), 6.95 (t, 1H, J=7.5), 7.02 (t, 1H, J=7.5), 7.06 (d, 1H, J=2.5), 7.23 (m, 2H), 7.32 (d, 1H, J=8.3), 7.35 (d, 1H, J=8.2), 10.31 (s, 1H), 10.96 (s, 1H); ¹³C NMR: 74.87, 109.56, 111.44, 115.41, 118.41, 120.29, 121.00, 121.63, 123.47, 124.72, 124.89, 128.99, 133.42, 136.77, 141.64, 178.41; EI (electron impact) m/z (%): 264.1 (25), 247.4 (47), 117.2 (37), 28.2 (100); HRMS (High Resolution Mass Spectroscopy): C₁₆H₁₂N₂O₂ requires 264.0899, found 264.0897.

### 5-bromo-3-(5-bromo-indol-3-yl)-3-hydroxy-indolin-2-one (9c)

Light yellow powder (5.40 g, 71%); mp 160° C. (dec.). ¹H NMR: 6.57 (s, 1H), 6.88 (d, 1H, J=8.2), 7.01 (d, 1H, J=2.5), 7.18 (dd, 1H, J=8.5, J=1.9), 7.35 (m, 2H), 7.47 (dd, 1H, J=8.4, J=2.1), 7.75 (d, 1H, J=1.9), 10.49 (s, 1H), 11.24 (s, 1H); ¹³C NMR: 74.66, 111.27, 111.78, 113.39, 113.63, 114.48, 122.97, 123.72, 125.12, 126.74, 127.39, 131.91, 135.24, 135.55, 140.92, 177.64.

### 3-(5-bromo-indol-3-yl)-3-hydoxy-indolin-2-one (9d)

Light orange crystals (5.55 g, 81%); mp 118° C. (dec.); ¹H NMR: 6.41 (s, 1H), 6.90 (d, 1H, J=7.5), 6.98 (d, 1H, J=2.5), 7.00 (t, 1H, J=7.6), 7.16 (dd, 1H, J=8.6, J=2.1), 7.27 (m, 2H), 7.32 (d, 1H, J=8.6), 7.71 (d, 1H, J=1.8), 10.33 (s, 1H), 11.18 (s, 1H); ¹³C NMR: 74.61, 109.64, 111.13, 113.49, 115.22, 121.76, 123.11, 123.56, 124.74, 125.04, 126.90, 129.20, 132.84, 135.50, 141.61, 178.16.

### 5-bromo-3-hydroxy-3-(5-methoxy-indol-3-yl)-indolin-2-one (9e)

Beige powder (0.773 g, 45%); mp 214° C. (dec.); ¹H NMR: 3.64 (s, 3H), 6.48 (s, 1H), 6.71 (dd, 1H, J=8.8, J=2.5), 6.85 (d, 1H, J=2.4), 6.88 (d, 1H, J=8.3), 7.03 (d, 1H, J=2.6), 7.24 (d, 1H, J=8.8), 7.34 (d, 1H, J=2.0), 7.44 (dd, 1H, J=8.2, J=2.1), 10.47 (s, 1H), 10.88 (d, 1H, J=1.7); ¹³C NMR: 55.15, 74.91, 102.38, 110.93, 111.63, 112.13, 113.27, 114.20, 124.24, 125.10, 127.37, 131.67, 131.96, 135.71, 140.93, 152.81, 177.82.

### 3-(5-bromo-indol-3-yl)-5-fluoro-3-hydoxy-indolin-2-one (9f)

Beige powder (5.37 g, 74%); mp 211° C. (dec.); ¹H NMR: 6.55 (s, 1H), 6.90 (dd, 1H, J=8.3, J=4.2), 7.01 (d, 1H, J=2.4), 7.11 (m, 2H), 7.17 (dd, 1H, J=8.6, J=2.0), 7.32 (d, 1H, J=8.6), 7.75 (d, 1H, J=1.8), 10.36 (s, 1H), 11.23 (s, 1H); ¹³C NMR: 74.91, 110.52, 110.58, 111.23, 112.23, 112.43, 113.57, 114.61, 115.38, 113.57, 123.10, 123.68, 125.15, 126.81, 134.50, 134.55, 135.54, 137.76, 157.09, 158.98, 178.10.

### 3-(5-bromo-indol-3-yl)-5-chloro-3-hydoxy-indolin-2-one (9g)

Light orange solid (5.59 g, 74%); mp 215° C. (dec.); ¹H NMR: 6.58 (s, 1H), 6.92 (d, 1H, J=8.3), 7.01 (d, 1H, J=2.5), 7.18 (dd, 1H, J=8.6, J=1.9), 7.26 (d, 1H, J=2.1), 7.34 (m, 2H), 7.75 (d, 1H, J=1.8), 10.49 (s, 1H), 11.24 (d, 1H, J=1.4); ¹³C NMR: 74.64, 111.178, 111.20, 113.55, 114.39, 122.94, 123.65, 124.61, 125.07, 125.68, 126.69, 129.00, 134.77, 135.48, 140.43, 177.71.

### 5-bromo-3-hydroxy-3-(5-methyl-indol-3-yl)-indolin-2-one (9h)

Yellow solid (2.13 g, 86%); mp 205° (dec.); ¹H NMR: 2.29 (s, 3H), 6.46 (s, 1H), 6.88 (m, 2H), 7.00 (d, 1H, J=2.4), 7.23 (m, 2H), 7.32 (d, 1H, J=1.9), 7.43 (dd, 1H, J=8.3, J=2.1), 10.47 (s, 1H), 10.89 (d, 1H, J=1.6); ¹³C NMR: 21.31, 74.86, 111.20, 111.59, 113.17, 114.07, 119.71, 122.69, 123.46, 124.91, 126.77, 127.21, 131.55, 135.09, 135.81, 140.81, 177.83.

### 3-(5-bromo-indol-3-yl)-3-hydoxy-5-(trifluoromethoxy)-indolin-2-one (9i)

Light beige powder (0.802 g, 41%); mp 201° C. (dec.); ¹H NMR: 6.62 (s, 1H), 6.97 (s, 1H), 6.99 (d, 1H, J=7.4), 7.18 (dd, 1H, J=8.6, J=1.9), 7.24 (s, 1H), 7.32 (m, 2H), 7.73 (d, 1H, J=1.6), 10.53 (s, 1H), 11.25 (s, 1H); ¹³C NMR: 74.75, 110.71, 111.31, 113.65, 114.36, 118.27, 122.53, 123.09, 123.78, 125.16, 126.78, 134.40, 135.54, 140.80, 143.23, 178.06.

### 3-(5-Hydroxy-indol-3-yl)-5-bromo-3-hydroxy-indoline-2-one (9j)

Light beige solid (1.85 g, 57%); mp 215° C. (dec.); ¹H NMR δ: 6.41 (s, 1H), 6.57 (dd, 1H, J=8.7, J=2.3), 6.75 (d, 1H, J=2.2), 6.86 (d, 1H, J=8.2), 6.98 (d, 1H, J=2.6), 7.13 (d, 1H, J=8.6), 7.30 (d, 1H, J=2.0), 7.42 (dd, 1H, J=8.2, J=2.1), 8.57 (s, 1H), 10.45 (s, 1H), 10.71 (d, 1H, J=2.0); ¹³C NMR δ: 74.89, 104.23, 111.55, 111.66, 111.74, 113.20, 113.66, 123.79, 125.38, 127.16, 131.27, 131.54, 135.90, 140.85, 150.15, 177.90.

### 3-(6-Bromo-indol-3-yl)-3-hydoxy-indolin-2-one (9k)

Light beige crystals (1.46 g, 84%); mp 182° C. (dec.); ¹H NMR δ: 6.39 (s, 1H), 6.89 (d, 1H, J=7.4), 6.97 (dt, 1H, J=7.5, J=0.9), 7.02 (d, 1H, J=2.5), 7.04 (dd, 1H, J=8.5, J=1.8), 7.25 (m, 2H), 7.41 (d, 1H, J=8.5), 7.53 (d, 1H, J=1.8), 10.32 (s, 1H), 11.10 (d, 1H, J=1.6); ¹³C NMR δ: 74.62, 109.63, 113.85, 113.99, 115.76, 121.33, 121.69, 122.33, 124.05, 124.48, 124.68, 129.11, 133.00, 137.66, 141.58, 178.15.

### 3-(7-Bromo-indol-3-yl)-3-hydoxy-indolin-2-one (91)

Yellow solid (1.41 g, 88%); mp 190° C. (dec.); ¹H NMR δ: 6.44 (s, 1H), 6.85 (t, 1H, J=7.8), 6.91 (d, 1H, J=7.7), 6.97 (t, 1H, J=7.5), 7.07 (d, 1H, J=2.6), 7.26 (m, 3H), 7.39 (d, 1H, J=8.0), 10.36 (s, 1H), 11.21 (d, 1H, J=1.8); ¹³C NMR δ: 74.68, 104.06, 109.68, 116.93, 119.93, 119.97, 121.74, 123.66, 124.58, 124.71, 126.60, 129.20, 132.93, 134.96, 141.63, 178.07.

### (ii) Reduction to bis-indolyl Species (0a, 10c-1)

To a solution of 9 (4-16 mmol) in dry THF at 0° C. was added BH₃.THF (1.0 M, 2.5 eq.) dropwise over 10 min., causing the solution to turn from light yellow to orange. The solution was stirred at room temperature overnight, then quenched by the dropwise addition of MeOH (30 mL) over 10 min., causing the solution to change from orange back to yellow. The trimethyl borate formed in this step and the solvent were removed under vacuum. The yellow solid was washed with MeOH (30 mL), which was removed under vacuum along with any remaining trimethyl borate. The solid was recrystallized and/or purified by flash column chromatography, as indicated.

### 3,3′-bis-indolyl (0a)

Recrystallized from THF/CH₂Cl₂. Yellow or green crystals (3.61 g, 57%); mp 285-287° C. (lit. 285-287° C. [120]); ¹H NMR: 7.05 (t, 2H, J=7.1), 7.14 (t, 2H, J=7.4), 7.43 (d, 2H, J=8.0), 7.62 (d, 2H, J=2.3), 7.77 (d, 2H, J=8.0), 11.13 (s, 2H); ¹³C NMR: 110.62, 112.44, 119.67, 120.46, 122.06, 122.72, 126.97, 137.28; EI m/z (%): 232 (100); HRMS: C₁₆H₁₂N₂ requires 232.1000, found 232.1005.

### 5-bromo-3-(5-bromo-indol-3-yl)-indole (10c)

Purification by flash chromatography in 2:1 hexanes:EtOAc and subsequent recrystallization of impure fractions from MeOH/H₂O. White or yellow crystals (2.25 g, 58%); mp 201-203° C.; ¹H NMR: 7.27 (dd, 2H, J=8.6, J=1.8), 7.45 (d, 2H, J=8.5), 7.77 (d, 2H, J=2.2), 7.88 (s, 2H), 11.45 (s, 2H); ¹³C NMR: 108.64, 111.62, 113.62, 121.41, 123.79, 123.88, 127.73, 135.04; EI m/z (%): 392 (89), 390 (100), 388 (31), 310 (23), 195 (35); HRMS: C₁₆H₁₀Br₂N₂ requires 387.9211, found 387.9222.

### 5-bromo-3-(indol-3-yl)-indole (10d)

Recrystallization from EtOH, followed by flash chromatography of the filtrate (after reducing the volume) in 3:1 hexanes:EtOAc. Yellow solid (1.78 g, 48%); mp 206° C. (dec.); ¹H NMR: 7.07 (t, 1H, J=7.1), 7.15 (t, 1H, J=7.5), 7.26 (dd, 1H, J=8.6, J=1.8), 7.42 (d, 1H, J=8.6), 7.45 (d, 1H, J=8.1), 7.66 (d, 1H, J=2.3), 7.70 (d, 1H, J=2.3), 7.74 (d, 1H, J=7.9), 7.87 (s, 1H), 11.18 (s, 1H), 11.38 (s, 1H); ¹³C NMR: 108.80, 109.49, 111.43, 111.59, 113.54, 118.87, 119.31, 121.23, 121.56, 122.16, 123.51, 123.65, 125.91, 127.82, 135.00, 136.34.

### 5-bromo-3-(5-methoxy-indol-3-yl)-indole (10e)

Recrystallization from EtOH/THF, followed by flash chromatography of the filtrate (after reducing the volume) in 2:1 hexanes:EtOAc. Beige solid (0.773 g, 45%); mp 214° C. (dec.); ¹H NMR: 3.77 (s, 3H), 6.80 (dd, 1H, J=8.7, J=2.3), 7.16 (d, 1H, J=2.2), 7.25 (dd, 1H, J=8.6, J=1.8), 7.34 (d, 1H, J=8.7), 7.42 (d, 1H, J=8.6), 7.60 (d, 1H, J=2.4), 7.69 (d, 1H, J=2.3), 7.85 (d, 1H, J=1.6), 11.03 (s, 1H), 11.35 (s, 1H); ¹³C NMR: 55.29, 101.08, 108.59, 109.59, 111.35, 111.41, 112.24, 113.53, 121.59, 122.90, 123.40, 123.60, 126.18, 127.83, 131.49, 135.00, 153.42.

### 5-bromo-3-(5-fluoro-indol-3-yl)-indole (10f)

Purification by flash chromatography in 3:1 hexanes:EtOAc. Grey powder (0.940 g, 48%); mp 154-156° C.; ¹H NMR: 7.00 (dt, 1H, J=9.1 (t), J=2.5), 7.26 (dd, 1H, J=8.6, J=1.9), 7.44 (m, 3H), 7.74 (d, 1H, J=2.4), 7.77 (d, 1H, J=2.4), 7.87 (d, 1H, J=1.8), 11.30 (s, 1H), 11.41 (s, 1H); ¹³C NMR: 103.79, 103.98, 109.07, 109.11, 109.26, 109.47, 111.47, 112.43, 112.51, 113.51, 121.40, 123.54, 123.67, 124.21, 125.89, 125.97, 127.60, 132.96, 134.95, 156.13, 157.97.

### 5-bromo-3-(5-chloro-indol-3-yl)-indole (10 g)

Purification by flash chromatography in 3:1 hexanes:EtOAc. Light green solid (1.35 g, 65%); mp 198-200° C.; ¹H NMR: 7.15 (dd, 1H, J=8.6, J=2.0), 7.26 (dd, 1H, J=8.6, J=1.9), 7.43 (d, 1H, J=8.6), 7.47 (d, 1H, J=8.6), 7.73 (d, 1H, J=2.0), 7.76 (d, 1H, J=2.4), 7.77 (d, 1H, J=2.4), 7.87 (d, 1H, J=1.8), 11.41 (s, 1H), 11.43 (s, 1H); ¹³C NMR: 108.61, 108.67, 111.53, 113.08, 113.55, 118.34, 121.20, 121.35, 123.59, 123.72, 123.78, 123.98, 126.92, 127.63, 134.75, 134.97.

### 5-bromo-3-(5-methyl-indol-3-yl)-indole (10h)

Purification by flash chromatography in 3:1 hexanes:EtOAc. Yellow powder (0.769 g, 47%); mp 214-216° C.; ¹H NMR: 2.41 (s, 3H), 6.97 (d, 1H, J=8.3), 7.25 (d, 1H, J=8.3), 7.34 (d, 1H, J=8.1), 7.42 (d, 1H, J=8.5), 7.54 (s, 1H), 7.61 (s, 1H), 7.70 (s, 1H), 7.87(s, 1H), 11.04 (s, 1H), 11.36 (s, 1H); ¹³C NMR: 21.34, 108.30, 109.65, 111.29, 111.43, 113.53, 118.93, 121.59, 122.24, 122.88, 123.47, 123.63, 126.18, 127.34, 127.88, 134.73, 135.03.

### 5-bromo-3-(5-(trifluoromethoxy)-indol-3-yl)-indole (10i)

Purification by flash chromatography in 2:1 hexanes:EtOAc. Yellow powder (0.450 g, 63%); mp 125-127° C.; ¹H NMR: 7.12 (dd, 1H, J=8.8, J=1.0), 7.26 (dd, 1H, J=8.6, J=1.9), 7.43 (d, 1H, J=8.6), 7.53 (d, 1H, J=8.7), 7.62 (s, 1H), 7.72 (d, 1H, J=2.3), 7.81 (d, 1H, J=2.3), 7.84 (d, 1H, J=1.7), 11.42 (s, 1H), 11.49 (s, 1H); ¹³C NMR: 108.61, 109.49, 111.61, 112.71, 113.69, 114.96, 117.47, 119.49, 121.44, 121.52, 123.54, 123.83, 123.84, 124.71, 125.94, 127.70, 134.85, 135.07, 141.87, 141.88.

### 3-(5-Bromo-indol-3-yl)-indol-5-ol (10j)

Purification by flash chromatography in 1:1 hexanes:THF. Yellow solid (0.560 g, 33%); mp 183° C. (dec.); ¹H NMR δ: 6.67 (dd, 1H, J=8.6, J=2.2), 7.02 (d, 1H, J=2.1), 7.23 (m, 2H), 7.41 (d, 1H, J=8.6), 7.51 (d, 1H, J=2.4), 7.55 (d, 1H, J=2.3), 7.81 (d, 1H, J=0.9), 8.63 (s, 1H), 10.86 (s, 1H), 11.32 (s, 1H); ¹³C NMR δ: 103.14, 107.86, 109.85, 111.24, 111.50, 111.83, 113.45, 121.55, 122.63, 123.05, 123.51, 126.64, 127.83, 130.81, 134.89, 150.66.

### 6-Bromo-3-(indol-3-yl)-indole (10k)

Hot filtration and recrystallization from EtOH. Yellow crystals (0.578 g, 47%); mp 238° C. (dec.); ¹H NMR δ: 7.06 (t, 1H, J=7.3), 7.15 (t, 1H, J=7.4), 7.18 (dd, 1H, J=8.5, J=1.8), 7.45 (dd, 1H, J=8.1, J=0.7), 7.62 (m, 1H), 7.64 (d, 1H, J=2.0), 7.67 (d, 1H, J=1.7), 7.72 (d, 1H, J=8.5), 7.76 (d, 1H, J=7.9), 11.19 (s, 1H), 11.29 (s, 1H); ¹³C NMR δ: 109.43, 110.51, 112.01, 114.33, 114.47, 119.32, 119.87, 121.69, 121.77, 122.04, 122.50, 123.23, 125.50, 126.33, 136.79, 137.64.

### 7-Bromo-3-(indol-3-yl)-indole (10l)

Suspended solid in MeOH (20 mL) and sonicated for 1 hr., filtered and rinsed with MeOH. Yellow solid (0.831 g, 67%); mp 240° C. (dec.); ¹H NMR δ: 7.02 (t, 1H, J=7.7), 7.08 (t, 1H, J=7.1), 7.15 (t, 1H, J=7.1), 7.37 (d, 1H, J=7.4), 7.45 (d, 1H, J=8.1), 7.65 (m, 2H), 7.73 (d, 1H, J=7.9), 7.78 (d, 1H, J=7.9), 11.21 (s, 1H), 11.38 (s, 1H); ¹³C NMR δ: 104.35, 180.90, 111.18, 111.58, 118.93, 119.15, 119.32, 120.18, 121.26, 122.29, 122.96, 123.80, 125.93, 127.84, 134.58, 136.33.

### (iii) Conversion of 5-bromo-3-(5-bromo-indol-3-yl)-indole (10c) to 5-methoxy-3-(5-methoxy-indol-3-yl)-indole (0b)

As depicted in Scheme 1 (iii), to a solution of 10c (0.780 g, 2.0 mmol) in DMF (8 mL) was added NaOMe (25 wt. % in MeOH, 9.0 mL, 20 eq.) and CuI (1.53 g, 4 eq.). After heating the suspension at reflux for 18 hrs., NH₃ (aq., 15 mL) was added and the aqueous layer extracted three times with EtOAc (10 mL). The organic layer was dried with MgSO₄, concentrated to ˜1 mL and purified by flash column chromatography using 2:1 hexanes:EtOAc as the solvent system, giving 0b as a grey powder (0.108 g, 19%).

### 5-methoxy-3-(5-methoxy-indol-3-yl)-indole (0b)

Grey powder (0.108 g, 19%); mp 193-196° C.; ¹H NMR: 3.32 (s, 6H), 6.78 (dd, 2H, J=8.7, J=2.2), 7.17 (d, 2H, J=2.0), 7.33 (d, 2H, J=8.7), 7.55 (d, 2H, J=2.2), 10.95 (s, 2H); ¹³C NMR: 101.12, 109.53, 111.30, 112.10, 122.44, 126.29, 131.44, 153.25; EI m/z (%): 292 (100), 262 (5.5); HRMS: C₁₈H₁₆N₂O₂ requires 292.1212, found 292.1223.

### (iv) Carboxylation of bromo-bis-indolyl Species (2b, 2c, 0c-1)

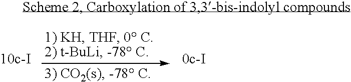

As depicted in Scheme 2, the bromo-indole species (0.8-3.0 mmol) was dissolved in dry THF (10 mL) and added dropwise to a suspension of KH (2.2 eq., 35 wt. % in oil) in THF (20 mL) at 0° C. The addition usually caused the reaction mixture to turn dark blue, regardless of initial colour. After 20 min., the reaction was cooled to −78° C. and t-BuLi (3 eq., 1.7M in pentane) was added dropwise, causing the reaction to turn a butterscotch colour. After a further 20 min. of stirring, a large excess of dry ice was added. The reaction was quenched after a final 20 min. period of stirring by adding MeOH (5 mL), followed by water (10 mL) and HCl (1N) until a pH of 2 was reached. The aqueous layer was extracted with EtOAc (2×20 mL) and the organic layer dried with Na₂SO₄. Products were purified by flash column chromatography using, as eluents, hexanes/EtOAc or hexanes/THF, both containing 2-5% AcOH. Other than 0e, products were isolated as 1:1 adducts with AcOH.

### 3-(5-bromo-indol-3-yl)-indole-5-carboxylic Acid (0c) AcOH

Purification by flash chromatography using 2:1 hexanes:EtOAc, 2% AcOH. Beige solid (0.138 g, 39%); mp 225° C. (dec.); ¹H NMR: 1.91 (s, 3H), 7.26 (dd, 1H, J=8.6, J=1.7), 7.45 (d, 1H, J=8.6), 7.50 (d, 1H, J=8.5), 7.69 (d, 1H, J=2.3), 7.77 (m, 2H), 7.85 (s, 1H), 8.38 (s, 1H), 11.44 (s, 1H), 11.56 (s, 1H), 12.19 (bs, 2H); ¹³C NMR: 20.95, 108.64, 110.08, 111.29, 111.57, 113.62, 121.36, 121.44, 121.86, 122.50, 123.74, 123.77, 123.83, 125.58, 127.76, 134.99, 138.71, 168.31, 171.87; EI m/z (%): 355 (0.9), 312 (100), 310 (73); HRMS: C₁₇H₁₁BrN₂O₂ requires 355.0004, found 355.0010.

### 3-(indol-3-yl)-indole-5-carboxylic Acid (0d).AcOH

Purification by flash chromatography using 2:1 hexanes:THF, 5% AcOH. Yellow powder (0.510 g, 60%); mp 226° C. (dec.); ¹H NMR: 1.91 (s, 3H), 7.07 (t, 1H, J=7.1), 7.16 (t, 1H, J=7.1), 7.47 (d, 1H, J=8.1), 7.50 (d, 1H, J=8.5), 7.61 (d, 1H, J=2.4), 7.71 (d, 1H, J=2.3), 7.74 (d, 1H, J=7.9), 7.77 (dd, 1H, J=8.5, J=1.5), 8.41 (s, 1H), 11.20 (s, 1H), 11.52 (s, 1H), 12.24 (bs, 2H); ¹³C NMR: 20.98, 108.86, 110.97, 111.20, 111.57, 118.86, 119.29, 121.24, 121.33, 122.11, 122.13, 122.41, 123.32, 125.68, 125.96, 136.31, 138.70, 168.39, 171.91; EI m/z (%): 276.0 (100); HRMS: C₁₇H₁₂N₂O₂ requires 276.0899, found 276.0902.

### 3-(5-methoxy-indol-3-yl)-indole-5-carboxylic Acid (0e)

Purification by flash chromatography using 1.6:1 hexanes:THF, 5% AcOH. Beige powder (0.128 g, 36%); mp 170° C. (dec.); ¹H NMR: 3.76 (s, 3H), 6.81 (dd, 1H, J=8.7, J=2.4), 7.18 (d, 1H, J=2.3), 7.36 (d, 1H, J=8.7), 7.50 (d, 1H, J=8.5), 7.56 (d, 1H, J=2.4), 7.71 (d, 1H, J=2.2), 7.77 (dd, 1H, J=8.5, J=1.5), 11.06 (s, 1H), 11.49 (s, 1H), 12.36 (bs, 1H); ¹³C NMR: 55.25, 101.10, 108.77, 111.17, 111.24, 111.51, 112.31, 121.45, 122.26, 122.43, 122.89, 123.21, 125.73, 126.27, 131.75, 138.75, 153.44, 168.50; EI m/z (%): 306 (4.1), 276 (34), 205 (100); HRMS: C₁₈H₁₄N₂O₃ requires 306.1004, found 306.0999.

### 3-(5-fluoro-indol-3-yl)-indole-5-carboxylic Acid (0f).AcOH

Purification by flash chromatography using 2:1 hexanes:THF, 5% AcOH. Yellow powder (0.324 g, 61%); mp 225° C. (dec.); ¹H NMR: 1.91 (s, 3H), 7.01 (dt, 1H, J=9.0(t), J=2.5), 7.48 (m, 3H), 7.71 (d, 1H, J=2.4), 7.75 (d, 1H, J=2.2), 7.78 (dd, 1H, J=8.5, J=1.5), 8.40 (s, 1H), 11.32 (s, 1H), 11.55 (s, 1H), 12.21 (bs, 2H); ¹³C NMR: 21.08, 103.87, 104.06, 109.25, 109.29, 109.39, 109.60, 110.50, 111.30, 112.57, 112.65, 121.54, 122.00, 122.54, 123.49, 124.28, 125.59, 126.08, 126.15, 133.05, 138.76, 156.20, 158.04, 168.45, 171.99; EI m/z (%): 294 (0.5), 60 (100); HRMS: C₁₇H₁₁FN₂O₂ requires 294.0804, found 294.0801.

### 3-(5-chloro-indol-3-yl)-indole-5-carboxylic Acid (0g).AcOH

Purification by flash chromatography using 2:1 hexanes:THF, 5% AcOH. Beige solid (0.328 g, 59%); mp 270° C. (dec.); ¹H NMR: 1.91 (s, 3H), 7.16 (dd, 1H, J=8.6, J=2.0), 7.49 (d, 1H, J=8.6), 7.51 (d, 1H, J=8.5), 7.75 (m, 4H), 8.39 (s, 1H), 11.43 (s, 1H), 11.57 (s, 1H), 12.21 (bs, 2H); ¹³C NMR: 21.05, 108.83, 110.18, 111.34, 113.20, 118.42, 121.31, 121.56, 121.93, 122.57, 123.69, 123.75, 124.04, 125.62, 127.09, 134.83, 138.76, 168.42, 171.98; EI m/z (%): 310 (1.0), 205 (88), 60 (100); HRMS: C₁₇H₁₁ClN₂O₂ requires 310.0509, found 310.0506.

### 3-(5-methyl-indol-3-yl)-indole-5-carboxylic Acid (0h).AcOH

Purification by flash chromatography using 2.6:1 hexanes:THF, 5% AcOH. Beige powder (0.159 g, 45%); mp 215° C.; ¹H NMR: 1.91 (s, 3H), 2.41 (s, 3H), 6.98 (d, 1H, J=7.9), 7.36 (d, 1H, J=8.2), 7.50 (d, 1H, J=8.5), 7.54 (s, 1H), 7.56 (d, 1H, J=2.1), 7.71 (d, 1H, J=2.0), 7.77 (dd, 1H, J=8.5, J=1.1), 8.41 (s, 1H), 11.06 (s, 1H), 11.50 (s, 1H), 12.22 (bs, 2H); ¹³C NMR: 21.10, 21.33, 108.42, 111.18, 111.23, 111.32, 118.97, 121.46, 122.18, 122.23, 122.46, 122.93, 123.30, 125.78, 126.27, 127.38, 134.75, 138.73, 168.51, 172.01; EI m/z (%): 290 (100), 220 (27), 205 (83); HRMS: C₁₈H₁₄N₂O₂ requires 290.1055, found 290.1055.

### 3-(5-(trifluoromethoxy)-indol-3-yl)-indole-5-carboxylic Acid (0i).AcOH

Purification by flash chromatography, run twice, using 2:1 hexanes:THF, 5% AcOH. Yellow powder (0.137 g, 33%); mp 220° C. (dec.); ¹H NMR: 1.91 (s, 3H), 7.14 (dd, 1H, J=8.7, J=1.1), 7.51 (d, 1H, J=8.5), 7.56 (d, 1H, J=8.8), 7.63 (s, 1H), 7.78 (m, 3H), 8.39 (s, 1H), 11.52 (s, 1H), 11.57 (s, 1H), 12.25 (bs, 2H); ¹³C NMR: 21.08, 109.58, 110.11, 111.36, 111.61, 112.74, 114.98, 117.45, 119.47, 121.49, 121.69, 121.92, 122.61, 123.52, 123.70, 124.69, 125.60, 126.05, 134.86, 138.77, 141.89, 168.43, 172.00; EI m/z (%): 360 (5.5), 220 (19), 205 (38), 60 (100); HRMS: C₁₈H₁₁F₃N₂O₃ requires 360.0721, found 360.0721.

### 3-(5-Hydroxy-indol-3-yl)-indole-5-carboxylic Acid (0j)

Purification by flash chromatography using 1:1 hexanes:EtOAc, 5% AcOH. Brown solid (0.082 g, 28%); mp 85-88° C.; ¹H NMR δ: 6.67 (dd, 1H, J=8.6, J=2.3), 7.04 (d, 1H, J=2.3), 7.25 (d, 1H, J=8.6), 7.48 (m, 2H), 7.57 (d, 1H, J=2.2), 7.76 (dd, 1H, J=8.5, J=1.4), 8.38 (s, 1H), 8.64 (bs, 1H), 10.89 (d, 1H, J=1.8), 11.44 (s, 1H), 12.28 (bs, 1H); ¹³C NMR δ: 103.24, 108.12, 111.08, 111.41, 111.62, 111.90, 122.15, 122.48, 122.64, 122.80, 125.76, 126.82, 130.89, 138.56, 150.76, 168.94; ESI m/z (%): 291.1 [M−1](100).

### 3-(Indol-3-yl)-indole-6-carboxylic Acid (0k).AcOH

Purification by flash chromatography using 2:1 hexanes:EtOAc, 5% AcOH. Yellow solid (0.155 g, 37%); mp 205° C. (dec.); ¹H NMR δ: 1.91 (s, 3H), 7.07 (t, 1H, J=7.1), 7.15 (t, 1H, J=7.2), 7.45 (d, 1H, J=8.0), 7.68 (m, 2H), 7.78 (d, 1H, J=7.9), 7.83 (d, 1H, J=8.4), 7.88 (d, 1H, J=2.4), 8.11 (s, 1H), 11.21 (s, 1H), 11.54 (s, 1H), 12.30 (bs, 2H); ¹³C NMR δ: 21.08, 109.02, 110.26, 111.59, 113.65, 118.89, 119.13, 119.44, 119.77, 121.25, 122.14, 123.53, 125.42, 125.92, 129.08, 135.64, 136.35, 168.41, 172.00; EI m/z (%): 276 (3.5), 232 (100); HRMS: C₁₇H₁₂N₂O₂ calculated 276.0899, found 276.0901.

### 3-(Indol-3-yl)-indole-7-carboxylic Acid (0l)

Suspended and sonicated solid in EtOAc (5 mL), filtered, rinsed with EtOAc. Yellow solid (0.349 g, 63%); mp 245° C. (dec.); ¹H NMR: 7.07 (t, 1H, J=7.5), 7.18 (m, 2H), 7.46 (d, 1H, J=8.0), 7.62 (d, 1H, J=1.8), 7.67 (d, 1H, J=2.0), 7.71 (d, 1H, J=7.9), 7.82 (d, 1H, J=7.4), 8.04 (d, 1H, J=7.8), 11.09 (s, 1H), 11.22 (s, 1H), 13.10 (bs, 1H); ¹³C NMR: 108.87, 110.07, 111.62, 113.98, 118.35, 118.93, 119.19, 121.26, 122.26, 123.08, 124.10, 124.88, 126.00, 127.68, 135.11, 136.37, 168.18; EI m/z (%): 275.9 (100), 257.9 (28), 230.0 (31); HRMS: C₁₇H₁₂N₂O₂ calculated 276.0899, found 276.0902

### EXAMPLE 1A

### (v) Cleavage of Methoxyl Groups to Hydroxyl Groups (0m)

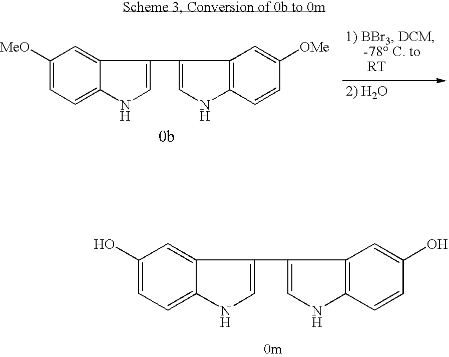

### 3-(5-hydroxy-indol-3-yl)-indol-5-ol (0m)

The product 0m was obtained by stirring 0b (0.923 g, 3.16 mmol) in dichloromethane (20 mL) at −78° C., to which was added BBr₃ (3 mL, 10 eq.). The dark red solution was allowed to slowly warm to room temperature and stirred for 20 h. The reaction mixture was then cooled to 0° C., water added (10 mL), and the pH raised to about 7 by adding 1N NaOH. The aqueous layer was extracted with EtOAc (2×50 mL) and the organic phase dried and concentrated, affording crude product. The product was purified by flash column chromatography, using 1:1 hexanes:EtOAc with 5% MeOH as the eluent. Yellow-green solid (0.546 g, 65%); mp 235° C. (dec.); ¹H NMR δ: 6.64 (dd, 2H, J=8.6, J=2.3), 7.03 (d, 2H, J=8.3), 7.21 (d, 2H, J=8.6), 7.36 (d, 2H, J=8.4), 8.57 (s, 2H), 10.76 (d, 2H, J=1.5); ¹³C NMR δ: 103.99, 109.69, 111.84, 112.17, 122.38, 127.30, 131.29, 150.96.

### EXAMPLE 2

### Synthesis of One Carbon-Linked Bis-Indoles

The one carbon-linked bis-indoles (1a-g) were synthesized from indole or 5-substituted indole and formaldehyde, using the method of Jackson et al. (J. Chem. Soc. Perkin Trans. I, 1987, 11: 2543-51 (Scheme 4).

Scheme 4 Synthesis of 1C bis-indoles. 1a and 1b produced
as a mixture, as were 1c and 1d.

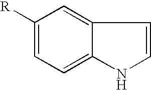

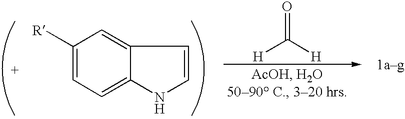

| R | R′ |
|---|---|
| H | — |
| OCH₃ | — |
| CO₂H | H |
| CO₂H | — |
| OH | — |

Formaldahyde (0.55 eq., 37% aqueous solution) and acetic acid (0.5 eq.) were added to indole or substituted indole (1.0-100 mmol) suspended in H₂O and the suspension heated at 90° C. for 8-20 hrs. (Scheme 4). Since 5-hydroxy-indole is sensitive to oxidation, the synthesis of 1 g was performed under argon and excluding light, and was heated at only 50° C. for 3 hrs. For the reaction giving 1a,b, as well as that for 1c,d, the beige, gummy mixture was collected by vacuum filtration, washed with water, recrystallized from EtOAc/hexanes and the resulting cream solid purified by flash chromatography using 4:1 hexanes/EtOAc as eluant. Purification of 1e-g are given below.

### Di-(indol-3-yl)methane (1a)

White crystals (5.55 g, 45%); mp 158-160° C.; ¹H NMR: 4.13 (s, 2H), 6.91 (t, 2H, J=7.3), 7.03 (t, 2H, J=7.3), 7.13 (s, 2H), 7.31 (d, 2H, J=8.2), 7.51 (d, 2H, J=7.6), 10.72 (s, 2H); ¹³C NMR: 20.94, 111.32 (2C), 114.21 (2C), 118.04 (2C), 118.69 (2C), 120.76 (2C), 122.77 (2C), 127.21 (2C), 136.41 (2C); EI m/z (%): 246.4 (6), 117.8 (100), 89.6 (49); HRMS: C₁₇H₁₄N₂ requires 246.1157, found 246.1147.

### 3-((indol-1-yl)methyl)-indole (1b)

White crystals (0.262 g, 2.1%); mp 82-84° C.; ¹H NMR: 5.50 (s, 2H), 6.38 (d, 1H, J=0.6), 7.02 (m, 4H), 7.34 (d, 1H, J=8.2), 7.49 (m, 4H), 7.64 (d, 1H, J=8.2), 11.03 (s, 1H); ¹³C NMR: 41.29, 100.30, 110.27, 111.02, 111.61, 118.56, 118.80 (2C), 120.38, 120.84, 121.29, 124.84, 126.50, 128.30, 128.84, 135.66, 136.36; EI m/z (%): 246.4 (8), 117.6 (100); HRMS: C₁₇H₁₄N₂ requires 246.1157, found 246.1168.

### Bis(5-methoxy-indol-3-yl)methane (1c)

Light grey powder (0.251 g, 45%); mp 167-169° C.; ¹H NMR: 3.71 (s, 6H), 4.07 (s, 2H), 6.70 (dd, 2H, J=8.8, J=2.4), 7.03 (d, 2H, J=2.4), 7.09 (d, 2H, J=2.4), 7.22 (d, 2H, J=8.5), 10.56 (s, 2H); ¹³C NMR: 20.90, 55.34 (2C), 100.72 (2C), 110.75 (2C), 111.93 (2C), 113.97 (2C), 123.50 (2C), 127.54 (2C), 131.58 (2C), 152.80 (2C); EI m/z (%): 306.4 (14), 147.6 (82), 104.6 (100); HRMS: C₁₉H₁₈N₂O₂ requires 306.1368, found 306.1358.

### 5-methoxy-3-((5-methoxy-indol-1-yl)methyl)-indole (1d)

Yellow crystals (0.062 g, 11%); mp 48-50° C.; ¹H NMR: 3.65 (s, 3H), 3.72 (s, 3H), 5.43 (s, 2H), 6.31 (dd, 1H, J=3.1, J=0.6), 6.73 (m, 2H), 6.96 (d, 1H, J=2.4), 7.01 (d, 1H, J=2.1), 7.24 (d, 1H, J=8.8), 7.40 (d, 1H, J=2.5), 7.46 (d, 1H, J=3.1), 7.53 (d, 1H, J=8.8), 10.86 (s, 1H); ¹³C NMR: 41.47, 55.31 (2C), 99.90, 100.69, 102.11, 110.92, 110.94, 111.15, 112.20, 125.24, 126.73, 128.69 (2C), 129.29, 131.06, 131.47, 153.35, 153.18; EI m/z (%): 306.4 (79), 160.5 (92), 147.5 (100); HRMS: C₁₉H₁₈N₂O₂ requires 306.1368, found 306.1370.

### 3-((indol-3-yl)methyl)-indole-5-carboxylic Acid (1e)

Purification by flash chromatography in 1.5:1 hexanes:THF, 5% AcOH, giving a light pink solid (0.045 g, 16%); mp 236-238° C.; ¹H NMR: 4.16 (s, 2H), 6.91 (t, 1H, J=7.5), 7.02 (t, 1H, J=7.5), 7.10 (d, 1H, J=2.2), 7.25 (d, 1H, J=2.0), 7.32 (d, 1H, J=8.1), 7.36 (d, 1H, J=8.3), 7.50 (d, 1H, J=8.0), 7.66 (dd, 1H, J=8.5, J=1.6), 8.19 (d, 1H, J=0.8), 10.74 (s, 1H), 11.10 (s, 1H), 12.25 (bs, 1H); ¹³C NMR: 20.79, 110.96, 111.30, 113.82, 115.57, 118.03, 118.57, 120.77, 121.39, 122.12, 122.70, 124.39, 126.68, 127.06, 136.38, 138.85, 168.47; EI m/z (%): 290 (5.8), 246 (91), 161 (100); HRMS: C₁₈H₁₄N₂O₂ requires 290.1055, found 290.1053.

### Bis-(indole-5-carboxylic acid-3-yl)methane (1 f)

Recrystallized from THF/H₂O, collected solid and purified filtrate by column chromatography in 1.5:1 hexanes:THF, 5% AcOH. White solid (0.152 g, 91%); mp 260° C. (dec.); ¹H NMR: 4.23 (s, 2H), 7.24 (d, 2H, J=2.0), 7.39 (d, 2H, J=8.6), 7.69 (dd, 2H, J=8.6, J=1.6), 8.19 (s, 2H), 11.14 (d, 2H, J=1.4), 12.31 (s, 2H); ¹³C NMR: 20.72, 111.07, 115.26, 120.67, 121.41, 122.19, 124.45, 126.87, 138.93, 168.37; EI m/z (%): 335 (M+1, 0.04), 161 (100), 144 (61); HRMS: C₁₉H₁₄N₂O₄ requires 334.0953, found 334.0933.

### Bis-(5-hydroxy-indol-3-yl)methane (1 g)

Purification by flash chromatography in 1:1 hexanes:EtOAc, 5% AcOH. Light pink solid (0.248 g, 50%); mp 57-59° C.; ¹H NMR: 3.92 (s, 2H), 6.55 (dd, 2H, J=8.6, J=2.3), 6.78 (d, 2H, J=2.3), 6.95 (d, 2H, J=2.2), 7.09 (d, 2H, J=8.6), 8.47 (s, 2H), 10.38 (d, 2H, J=1.3); ¹³C NMR: 21.14, 102.69, 110.98, 111.45, 113.09, 123.13, 127.84, 130.95, 149.84; EI m/z (%): 279 (M+1, 1.0), 74 (75), 59 (100); HRMS: C₁₇H₁₄N₂O₂ requires 278.1055, found 278.1054.

In addition to the methylene-linked bis-indoles, three bis-(5-hydroxyindole) compounds (1h,i,j) were generated which also had the aromatic groups separated by one carbon. These were obtained by condensing 5-hydroxyindole with one, two or three units of acetone in the presence of trifluoroacetic acid (Scheme 5), following the procedure used for unsubstituted indole. The one-acetone condensation product 1h was produced within seconds at room temperature, with the two- and three-acetone products (1i,j) becoming the dominant species after several hours and several days, respectively.

Scheme 5.
Condensation of 5-hydroxyindole with acetone to give 1h, i, j.

| | | Major species |
|---|---|---|
| | Time | (TLC) |
| 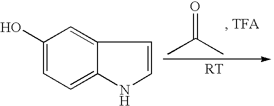 | 0 to˜6 hrs 6 hrs. to 3-5 days >5 days | 1h 1i 1j |

The highly branched bridging groups of 1h,i,j provide additional molecular diversity to help identify favourable characteristics for compound activity against Aβ. Furthermore, these compounds are expected to be more stable than the methylene-linked species 1a-g, which are likely easily oxidized at the CH₂ bridge. The observation that 1h,i,j remained a light colour when exposed to light and air, while 1a-g become darkly coloured within a few days or weeks, supports this notion.

Condensation of 5-hydroxyindole with Acetone (1h,i,j):

5-Hydroxyindole (0.133 g, 1.00 mmol) was stirred at room temperature in acetone (2 mL) and TFA (0.150 mL, 2.0 eq.). Upon completion (5 min. for 1h, or 18 h for a mixture of 1i and 1j), the reaction mixture was neutralized with saturated NaHCO₃(aq.), and the product(s) extracted with EtOAc (2×20 mL). Products were purified by flash column chromatography in 1.5:1 (1h) or 2:1 (1i,j) hexanes:EtOAc, both containing 5% MeOH.

### 2,2-Bis-(5-hydroxyindol-3-yl)propane (1h)

Light beige solid (0.099 g, 65%); mp 133-136° C.; ¹H NMR δ: 1.72 (s, 6H), 6.45 (dd, 2H, J=8.6, J=2.3), 6.53 (d, 2H, J=2.3), 7.05 (d, 2H, J=8.6), 7.07 (d, 2H, J=2.5), 8.27 (s, 2H), 10.38 (d, 2H, J=2.0); ¹³C NMR δ: 29.67, 33.91, 104.64, 110.58, 111.32, 121.17, 122.71, 126.57, 131.65, 149.04; EI m/z(%): 306.0(3.8), 133(12) 59 (100); HRMS: C₁₉H₁₈N₂O₂ calculated 306.1368, found 306.1374.

### 1,2,3,4-Tetrahydro-3-(5-hydroxyindol-3-yl)-1,1,3-trimethylcyclopenta[b]indol-7-ol (1i)

White solid (0.126 g, 73%); mp 132° C. (dec.); ¹H NMR δ: 1.35 (s, 3H), 1.42 (s, 3H), 1.71 (s, 3H), 2.36 (d, 1H, J=12.7), 2.75 (d, 1H, J=12.7), 6.48 (dd, 1H, J=8.6, J=2.6), 6.54 (dd, 1H, J=8.6, J=2.3), 6.56 (d, 1H, J=2.0), 6.79 (d, 1H, J=2.2), 6.91 (d, 1H, J=2.5), 7.00 (d, 1H, J=8.6), 7.10 (d, 1H, J=8.5), 8.45 (s, 2H), 10.28 (s, 1H), 10.45 (d, 1H, J=1.9); ¹³C NMR δ: 28.72, 30.04, 30.58, 38.13, 41.66, 61.81, 102.18, 103.73, 109.17, 110.96, 111.64, 111.92, 121.27, 121.76, 122.89, 123.60, 125.93, 131.58, 135.35, 148.70, 149.65, 150.05; EI m/z (%): 346 (22), 331 (74), 56 (100); HRMS: C₂₂H₂₂N₂O₂ calculated 346.1681, found 346.1690.

### 3,3,3′,3′-Bis(1,2,3,4-tetrahydro-1,1-dimethylcyclopenta[b]indol-7-ol) (1j)

White solid (0.042 g, 22%); mp 140° C. (dec.); ¹H NMR δ (acetone-d₆): 1.47 (s, 6H), 1.53 (s, 6H), 2.58 (d, 2H, J=12.9), 2.80 (d, 2H, J=12.9), 6.65 (dd, 2H, J=8.6, J=2.3), 6.95 (d, 2H, J=2.0), 7.12 (d, 2H, J=8.6), 7.54 (s, 2H), 9.50 (s, 2H); ¹³C NMR δ (acetone-d₆): 31.05, 31.58, 40.39, 50.89, 63.97, 104.2, 111.5, 113.6, 125.89, 127.43, 138.15, 147.82, 152.30; EI m/z (%):386 (14), 198 (64), 174 (100); HRMS: C₂₅H₂₆N₂O₂ calculated 386.1994, found 386.1995.

### EXAMPLE 3

### Synthesis of Two Carbon-Linked Bis-Indoles

Directly linked bis-indoles were synthesized according to the procedure set forth in Scheme 6 below:

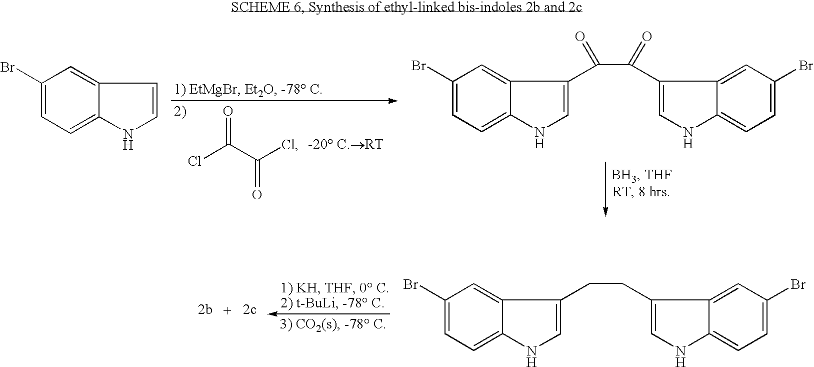

### (i) Formation of 3,3′-oxalyl-linked bis-(5-bromo-indole)

A suspension of 5-bromo-indole (3.92 g, 20 mmol) in Et₂O (100 mL) was cooled to −20° C. and ethylmagnesium bromide (7.3 mL, 3.0 M in THF, 1.1 eq.) added dropwise. The beige suspension was warmed to room temperature for 3 hrs. before again cooling to −20° C. and adding oxalyl chloride (0.87 mL, 0.50 eq.) dropwise, causing an oily orange/brown precipitate to form. After stirring for 3 hrs., water (50 mL) was added and the suspension sonicated for 30 min. A yellow solid was collected by vacuum filtration, suspended in MeOH (30 mL) and stirred overnight to dissolve impurities before again being collected by vacuum filtration.

### 1,2-bis(5-bromo-indol-3-yl)ethane-1,2-dione

Yellow solid (0.900 g, 20%); mp N.D.; ¹H NMR: 7.44 (dd, 2H, J=8.5, J=2.0), 7.35 (d, 2H, J=8.5), 8.35 (d, 2H, J=3.5), 8.43 (d, 2H, J=1.5), 12.42 (s, 2H); ¹³C NMR: 107.60, 112.44, 115.30, 115.88, 124.11, 126.72, 128.11, 136.13, 139.22, 187.60; EI m/z (%): 446 (13), 232 (100), 222 (39); HRMS: C₁₈H₁₀Br₂N₂O₂ requires 443.9109, found 443.9114.

### (ii) Reduction to ethyl-linked bis-(5-bromo-indole)

To a solution of 1,2-bis(5-bromo-indol-3-yl)ethane-1,2-dione (0.900 g, 2.03 mmol) in THF (20 mL) was added BH₃.THF (10.8 mL, 1.0 M in THF, 5 eq.). The solution was stirred at room temperature for 8 hrs. before slowly quenching the reaction with MeOH (20 mL). The solvent and the B(OMe)₃ formed were removed under vacuum and the remaining solid sonicated with MeOH (10 mL). The MeOH and any remaining B(OMe)₃ were removed and the solid purified by flash chromatography using 3.5:1 CHCl₃:hexanes as the solvent system. The product, 1,2-bis(5-bromo-indol-3-yl)ethane, was obtained as a white powder and used without further purification.

### (iii) Carboxylation to 2b and 2c

The carboxylation of 1,2-bis(5-bromo-indol-3-yl)ethane to 2b and 2c was carried out as described in Example 1, paragraph iv. After adding CO₂(s), the reaction was quenched with MeOH (5 mL) and H₂O (20 mL), and the basic solution extracted twice with EtOAc (20 mL) before acidifying to pH 3 with 1 N HCl. TLC suggested that the resulting white precipitate was primarily a mixture of 2b and 2c (i.e. the mono- and dicarboxylated species). These were separated by flash chromatography in 2:1 hexanes:THF, 5% AcOH to isolate 2b, followed by 1:1 hexanes:THF, 5% AcOH, to isolate 2c. Purification of 2b was achieved by recrystallizing from THF/hexanes, while 2c was purified by refluxing in EtOH (10 mL) for 30 min. before being collected by vacuum filtration.

### 3-(2-(5-bromo-indol-3-yl)ethyl)-indole-5-carboxylic Acid (2b)

Light yellow powder (0.062 g, 16%); mp 205° C. (dec.); ¹H NMR: 3.07 (m, 4H), 7.16 (dd, 1H, J=8.6, J=1.8), 7.24 (m, 2H), 7.30 (d, 1H, J=8.6), 7.36 (d, 1H, J=8.5), 7.67 (d, 1H, J=1.7), 7.72 (d, 1H, J=8.5), 8.25 (s, 1H), 11.03 (s, 1H), 11.09 (s, 1H); ¹³C NMR: 25.41, 25.44, 110.73, 110.77, 113.28, 114.46, 115.89, 120.57, 120.79, 122.30, 123.18, 123.65, 124.00, 126.66, 129.06, 134.85, 138.40, 169.06; EI m/z (%): 382 (0.5), 338 (8.0), 130 (100); HRMS: C₁₉H₁₅BrN₂O₂ requires 382.0317, found 382.0314.

### 1,2-bis-(indole-5-carboxylic acid-3-yl)ethane (2c)

White powder (0.031 g, 9%); mp 255° C. (dec.); ¹H NMR: 3.13 (s, 4H), 7.29 (d, 2H, J=2.1), 7.40 (d, 2H, J=8.5), 7.71 (dd, 2H, J=1.3), 8.25 (s, 2H), 11.15 (s, 2H), 12.30 (s, 2H); ¹³C NMR: 25.33, 111.02, 116.02, 120.73, 121.01, 122.18, 123.90, 126.73, 138.70, 168.45.

### EXAMPLE 4

### Synthesis of 3-(7-azaindol-3-yl)-indoles

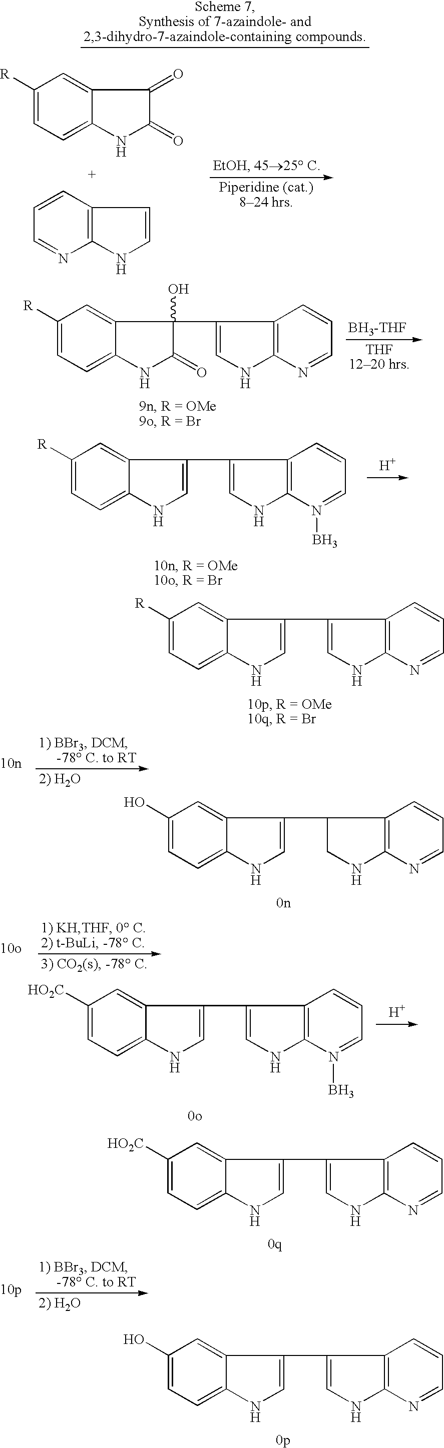

### 3-(5-methoxyindol-3-yl)-7-azaindole.BH₃ (10n)

The procedure from Example 1, paragraph (i) was used to give 9n (Scheme 6), a light orange solid that was rinsed with EtOH (5 mL) and dried under vacuum (2.52 g, 85%). The procedure from Example 1, paragraph (ii) was used to give 10n from 9n. Purification of 10n was achieved by flash column chromatography using 1.7:1 hexanes:EtOAc as the eluent. Finally, the product was recrystallized from EtOH, giving yellow crystals (1.22 g, 58%). ¹H NMR (DMSO-d₆): 2.58 (bs, 3H), 3.78 (s, 3H), 6.83 (dd, 1H, J=8.8, J=2.4), 7.16 (d, 1H, J=2.3), 7.35 (m, 2H), 7.72 (d, 1H, J=2.5), 7.78 (d, 1H, J=2.4), 8.32 (d, 1H, J=5.5), 8.51 (d, 1H, J=7.8), 11.22 (s, 1H), 11.94 (s, 1H); ¹³C NMR: 55.86, 56.51, 101.41, 107.53, 111.21, 112.94, 115.79, 122.67, 123.97, 124.30, 126.51, 132.03, 132.77, 141.27, 143.06, 154.25.

### 3-(5-bromoindol-3-yl)-7-azaindole.BH₃ (10o)

Same procedure as for 10n (Scheme 6). Purification after the coupling step was accomplished by flash column chromatography using 2:1 EtOAc:hexanes, 5% MeOH as the eluent, giving a yellow-orange solid (2.40 g, 97%). Purification of 10o after BH₃ reduction was accomplished by flash column chromatography using 1.2:1 hexanes:EtOAc, giving a bright yellow solid (1.13 g, 54%). ¹H NMR (DMSO-d₆): 2.58 (bs, 3H), 7.29 (dd, 1H, J=8.6, J=1.8), 7.36 (dd, 1H, J=7.9, J=5.6), 7.45 (d, 1H, J=8.6), 7.85 (m, 3H), 8.33 (d, 1H, J=5.5), 8.52 (d, 1H, J=7.9), 11.60 (s, 1H), 12.02 (s, 1H); ¹³C NMR: 107.52, 110.22, 112.47, 114.24, 115.91, 121.70, 122.48, 124.45, 124.60, 125.23, 127.90, 132.64, 135.54, 141.37, 143.01.

### 3-(5-methoxyindol-3-yl)-7-azaindole (10p)

To remove the BH₃ group, crude 10n (approx. 1.5 g, 6 mmol) was sonicated in a mixture of AcOH and 1N HCl (1:1, 50 mL) until TLC indicated completion (1 hr.). The reaction was neutralized with saturated Na₂CO₃ (aq.) until the pH reached 7-8, and extracted with EtOAc (2×20 mL). The organic phase was dried (Na₂SO₄) and concentrated, and the product (10n) purified by recrystallization from THF/hexanes. Further product was obtained from the filtrate after purifying by column chromatography using 1:1 hexanes:THF as eluent. Yellow solid, 1.45 g (approx. 100%). ¹H NMR (DMSO-d₆): 3.34 (s, 3H), 6.80 (dd, 1H, J=8.8, J=2.3), 7.11 (dd, 1H, J=7.8, J=4.6), 7.21 (d, 1H, J=2.0), 7.33 (d, 1H, J=8.7), 7.64 (d, 1H, J=2.3), 7.76 (d, 1H, J=2.2), 8.16 (d, 1H, J=7.8), 8.26 (d, 1H, J=4.6), 11.07 (s, 1H), 11.68 (s, 1H); ¹³CNMR: 55.32, 101.17, 108.77, 108.78, 111.47, 112.23, 115.25, 118.20, 121.78, 122.84, 126.01, 127.69, 131.50, 142.64, 148.69, 153.49.

### 3-(2,3-dihydro-7-azaindol-3-yl)-indol-5-ol (0n)

Conversion of 10n to 0n was carried out using the procedure from Example 1, paragraph (v). In the course of the reaction, however, the 2,3 double bond of 7-azaindole was reduced during the reaction by the BH₃ present. The product was purified by chromatography using 12:1 CHCl₃:MeOH as the eluent, affording 0n as a light yellow solid (0.610 g, 61%). ¹H NMR (DMSO-d₆): 3.41 (t, 1H, J=9.1), 3.73 (t, 1H, J=9.1), 4.57 (t, 1H, J=9.2), 5.08 (s, 1H), 6.29 (d, 1H, J=1.9), 6.39 (d, 1H, J=2.4), 6.41 (d, 1H, J=2.3), 6.96 (dd, 1H, J=7.8, J=4.7), 7.30 (s, 1H), 7.76 (d, 1H, J=7.6), 8.17 (dd, 1H, J=4.6, J=1.2), 8.31 (d, 1H, J=4.1), 11.39 (s, 1H); ¹³C NMR: 40.44, 55.14, 110.37, 112.21, 113.99, 115.27, 115.72, 118.91, 123.28, 127.73, 133.76, 142.93, 144.92, 149.47, 150.20.

### 3-(7-azaindol-3-yl)-indole-5-carboxylic acid.BH₃ (0o)

Carboxylation of 10o to 0c followed the same procedure as for 0c-1 (Example 1, paragraph (iv)). Purification of the product was achieved by recrystallization from EtOH/H₂O, giving a yellow solid (0.383 g, 45%). ¹H NMR (DMSO-d₆): 2.58 (bs, 3H), 7.38 (dd, 1H, J=7.9, J=5.6), 7.53 (d, 1H, J=8.6), 7.82 (m, 2H), 7.93 (d, 1H, J=2.4), 8.36 (d, 1H, J=5.5), 8.43 (s, 1H), 8.56 (d, 1H, J=7.8), 11.77 (d, 1H, J=1.5), 12.07 (s, 1H), 12.53 (s, 1H); ¹³C NMR: 108.56, 109.88, 111.57, 115.51, 121.50, 121.92, 121.99, 122.85, 123.83, 124.69, 125.30, 132.27, 138.83, 141.00, 142.54, 168.29.

### 3-(7-azaindol-3-yl)-indole-5-carboxylic acid.AcOH (0p)

Conversion of 0o to 0p was the same as for 10p. Purification of the product was achieved by column chromatography, using 1.2:1 hexanes:THF, 2% AcOH as the eluent, giving a yellow solid (0.125 g, approx. 100%). ¹H NMR (DMSO-d₆): 1.92 (s, 3H), 7.14 (dd, 1H, J=7.9, J=4.6), 7.51 (d, 1H, J=8.6), 7.74 (d, 1H, J=2.4), 7.78 (m, 2H), 8.16 (dd, 1H, J=7.9, J=1.2), 8.29 (dd, 1H, J=4.6, J=1.4), 8.42 (d, 1H, J=0.6), 11.61 (s, 1H), 11.79 (s, 1H), 12.20 (bs, 2H); ¹³C NMR: 21.03, 107.94, 110.28, 111.37, 115.47, 118.21, 121.59, 121.99, 122.30, 122.61, 123.75, 124.88, 125.48, 138.79, 142.87, 148.67, 168.39, 171.97.

### 3-(7-azaindol-3-yl)-indol-5-ol (0p)

Conversion of 10p to 0p was carried out using the procedure from Example 1, paragraph (v). The product was purified by chromatography using 8:1 CHCl₃:MeOH as the eluent, affording 0p as a yellow solid (0.483 g, 65%). ¹H NMR (DMSO-d₆): 6.68 (dd, 1H, J=8.6, J=2.2), 7.10 (m, 2H), 7.23 (d, 1H, J=8.6), 7.56 (d, 1H, J=2.4), 7.60 (d, 1H, J=2.3), 8.14 (d, 1H, J=7.8), 8.25 (d, 1H, J=4.6), 8.65 (s, 1H), 10.90 (s, 1H), 11.62 (s, 1H); ¹³C NMR: 103.31, 108.08, 109.15, 111.60, 111.90, 115.19, 118.21, 121.35, 122.60, 126.49, 127.79, 130.89, 142.59, 148.59, 150.79.

### EXAMPLE 5

### Synthesis of Indole-Phenyl Compounds

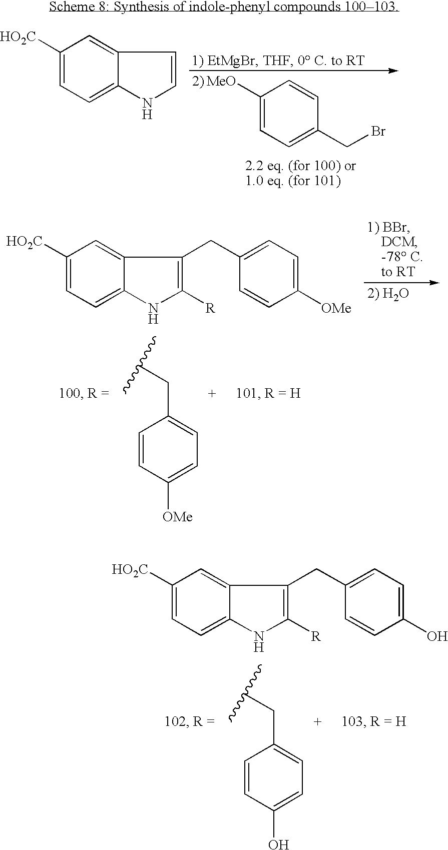

### 2,3-bis(4-methoxybenzyl)-indole-5-carboxylic Acid (100)

To solution of indole-5-carboxylic acid (0.322 g, 2.0 mmol) in THF (20 mL) at 0° C. was added EtMgBr (2.2 eq., 3.0 M solution in THF), giving a gummy white suspension. 1-(Bromoethyl)-4-methoxybenzene (2.2 eq.) was added dropwise and the suspension warmed to room temperature and stirred overnight. Water was added (10 mL), followed by 1 N HCl (aq) until the pH was 2. The reaction mixture was extracted with EtOAc (20 mL×2) and the organic phase dried with MgSO₄. Purification of 101 was achieved by column chromatography using 4:1 hexanes:EtOAc, 3% AcOH as the eluent, followed by recrystallization from EtOH/H₂O. The product was obtained as a white solid (0.345 g, 43%). ¹H NMR (DMSO-d₆): 3.68 (s, 3H), 3.70 (s, 3H), 4.04 (s, 2H), 4.05 (s, 2H), 6.78 (d, 2H, J=8.6), 6.83 (d, 2H, J=8.6), 7.06 (d, 2H, J=8.5), 7.13 (d, 2H, J=8.5), 7.30 (d, 1H, J=8.5), 7.62 (dd, 1H, J=8.5, J=1.2), 7.96 (s, 1H), 11.14 (s, 1H), 12.22 (s, 1H); ¹³C NMR: 28.93, 31.22, 55.43, 55.53, 110.85, 111.65, 114.13, 114.33, 121.24, 121.44, 122.35, 128.09, 129.46, 129.91, 131.75, 133.95, 137.41, 138.72, 157.81, 158.23, 168.89.

### 2,3-bis(4-hydroxybenzyl)-indole-5-carboxylic Acid (102)

Conversion of 100 to 102 was carried out using the same procedure as for 0m (Example 1, paragraph (v)). The product was purified by column chromatography using 1.3:1 hexanes:EtOAC, 5% AcOH as the eluent, giving a light pink solid (0.190 g, 85%). ¹H NMR (DMSO-d₆): 3.97 (s, 2H), 3.99 (s, 2H), 6.60 (d, 2H, J=8.2), 6.65 (d, 2H, J=8.2), 6.94 (d, 2H, J=8.3), 7.00 (d, 2H, J=8.3), 7.28 (d, 1H, J=8.5), 7.61 (d, 1H, J=8.5), 7.96 (s, 1H), 9.10 (bs, 2H), 11.08 (s, 1H), 12.21 (bs, 1H); ¹³C NMR: 29.00, 31.27, 110.78, 111.78, 115.45, 115.62, 121.23, 121.27, 122.26, 128.16, 129.41, 129.84, 129.96, 132.18, 137.52, 138.67, 155.71, 156.19, 168.93.

### 3-(4-hydroxybenzyl)-indole-5-carboxylic Acid (103)

101 was synthesized in the same manner as 100, using only 1.0 eq. of 1-(bromoethyl)-4-methoxybenzene instead of 2.2 eq., and 103 was made from 101 using the same procedure as for 102. The product (103) was purified by column chromatography using 1.6:1 hexanes:EtOAc, 5% AcOH as the eluent, followed by recrystallization from EtOH/H₂O, giving cream-colored needles (0.110 g, 33%). ¹H NMR (DMSO-d₆): 3.96 (s, 2H), 6.65 (d, 2H, J=8.4), 7.05 (d, 2H, J=8.4), 7.21 (d, 1H, J=1.8), 7.38 (d, 1H, J=8.5), 7.68 (dd, 1H, J=8.5, J=1.5), 8.09 (s, 1H), 9.11 (s, 1H), 11.16 (s, 1H), 12.31 (s, 1H); ¹³C NMR: 30.41, 111.55, 115.50, 116.45, 121.27, 121.84, 122.72, 125.11, 127.04, 129.65, 131.92, 139.42, 155.80, 168.87.

### EXAMPLE 6

### Suzuki Coupling of Various Bicyclic Aromatics

### (i) Synthesis of 3-(Benzofuran-2-yl)-indole-5-carboxylic acid (57) and 3-(Benzothiophen-2-yl)-indole-5-carboxylic acid (58)

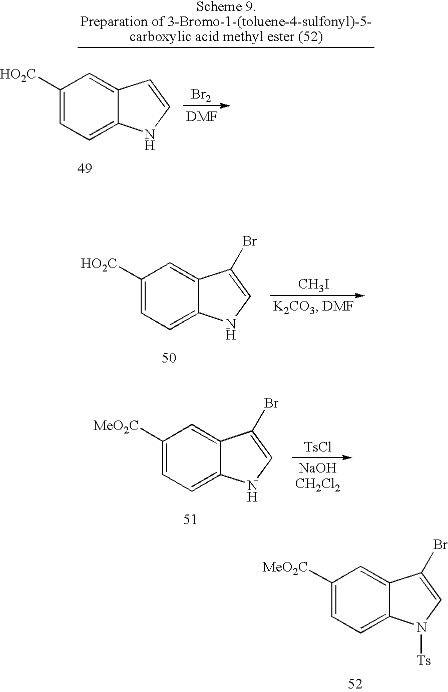

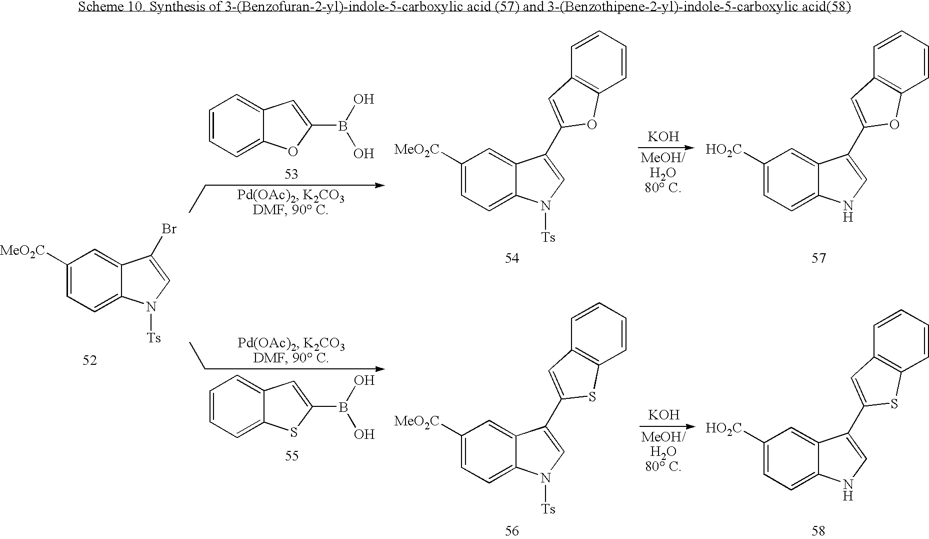

### 3-Bromo-indole-5-carboxylic Acid (50)

To a solution of commercially available indole-5-carboxylic acid (1.0 g, 6.2 mmol) in DMF (10 mL) was added Br₂ (334 μL, 1.05 eq.) at room temperature (Scheme 9). Upon completion (˜5 min), the reaction mixture was poured to an ice-cold solution of Na₂SO₃ (1% in H₂O, 100 mL), resulting to the precipitation of the product, which was filtered and dried under high vacuum pressure. The isolated product was used without further purification. Light beige solid (1.46 g, 98%). ¹H NMR (DMSO): 7.47 (d, 1H, J=8.6), 7.65 (d, 1H, J=2.5), 7.76 (d, 1H, J=7.3, 1.3), 8.04 (s, 1H), 11.77 (s, 1H), 12.57 (s, 1H).

### 3-Bromo-indole-5-carboxylic Acid Methyl Ester (51)

To a mixture of 50 (1.0 g, 4.2 mmol) and K₂CO₃ (0.865 g, 1.5 eq.) in DMF (15 mL) was added CH₃I (337 μL, 1.3 eq.) at room temperature (Scheme 9). After stirring for 1.2 hours, the reaction was quenched with a saturated solution of NH₄Cl (10 mL). The aqueous layer was extracted with ethyl acetate (3×15 mL) and the combined organic layer was dried with MgSO₄, filtered and concentrated under reduced pressure. The residue was purified by flash column chromatography (15% ethyl acetate in hexane) to afford 51 (1.01 g, 95%). ¹H NMR (DMSO): 3.87 (s, 3H), 7.53 (d, 1H, J=8.6), 7.71 (s, 1H), 7.35 (dd, 1H, J=8.6, 1.5), 7.64(s, 1H), 11.41 (s, 1H).

### 3-Bromo-1-(toluene-4-sulfonyl)-indole-5-carboxylic Acid Methyl Ester (52)

To a mixture of 51 (0.800 g, 3.15 mmol) and powdered NaOH (0.139 g, 1.1 eq.) in CH₂Cl₂ (25 mL) was added p-toleuenesulfonyl chloride (0.661 g, 1.1 eq.) at room temperature (Scheme 9). After stirring for 2.5 hours, the reaction was quenched with saturated solution of NH4Cl (20 mL). Layers were separated. The aqueous layer was extracted with CH₂Cl₂ (3×15 mL). The combined organic layer was dried with MgSO₄, filtered and concentrated under reduced pressure. Purification by flash column chromatography using 10% ethyl acetate in hexane as solvent system afforded 52 (1.081 g, 84%) as light orange solid. ¹H NMR (DMSO): 2.34 (s, 3H), 3.89 (s, 3H), 7.43 (d, 2H, J=8.3), 7.97 (s, 1H), 7.98 (s, 1H) 8.03-8.05 (m, 2H), 8.15 (d, 1H, J=9.14), 8.30 (s, 1H).

### Suzuki Coupling of 3-bromo-1-(toluene-4-sulfonyl)-indole-5-carboxylic Acid Methyl Ester (52) with benzofuran-2-boronic Acid (53) to Give 54

To a degassed solution of 52 (0.200 g, 0.489 mmol) was added Pd(OAc)₂ (0.006 g, 0.05 eq.), K₂CO₃ (0.135 g, 2 eq.) and benzofuran-2-boronic acid 53 (0.103 g, 1.3 eq.) at room temperature (Scheme 10). After degassing and purging with argon (repeated thrice), the reaction mixture was stirred at 90° C. for 2.5 hours. The mixture was allowed to cool to room temperature and diluted with H₂O (15 mL). The aqueous solution was extracted with ethyl acetate (5×15 mL) and combined organic layer was concentrated under reduced pressure. The residue was purified by flash column chromatography using 20% ethyl acetate in hexane as solvent system to afford 54 (0.168 g, 77.1%) as white solid. ¹H NMR (CDCl₃) 2.34 (s, 3H), 3.97 (s, 3H), 7.11 (s, 1H), 7.23-7.27 (m, 3H), 7.31 (t, 1H, J=7.3), 7.54 (d, 1H, J=8.1), 7.61 (d, 1H, J=7.6), 7.84 (d, 2H, J=8.2), 8.09 (2s, 2H), 8.13 (s, 1H), 8.65 (s, 1H); ¹³C NMR (CDCl₃) 21.64, 51.88, 103.27, 110.71, 113.63, 114.29, 120.95, 123.18, 124.65, 124.88, 126.08, 126.53, 127.05, 127.21, 128.88, 130.21, 134.74, 137.85, 145.76, 149.39, 154.30, 166.38

### Suzuki coupling of 1-benzenesulfonyl-3-bromo-indole-5-carboxylic Acid Methyl Ester (52) with benzothiophene-2-boronic Acid (55) to give 56

To a degassed solution of 52 (0.200 g, 0.489 mmol) was added Pd(OAc)₂ (0.006 g, 0.05 eq.), K₂CO₃ (0.135 g, 2 eq.) and benzothiophene-2-boronic acid 55 (0.105 g, 1.2 eq.) at room temperature (Scheme 10). After degassing and purging with argon (repeated thrice), the reaction mixture was stirred at 90° C. for 12 hours. The mixture was allowed to cool to room temperature and diluted with H₂O (15 mL). The aqueous solution was extracted with ethyl acetate (5×15 mL) and combined organic layer was concentrated under reduced pressure. The residue was purified by flash column chromatography using 20% ethyl acetate in hexane as solvent system to afford 56 (0.180 g, 82.7%) as white solid. ¹H NMR (CDCl₃): 2.36 (s, 3H), 3.95 (s, 3H), 7.26 (d, 2H, J=8.5), 7.30-7.41 (m, 2H), 7.62 (s, 1H), 7.82-7.95 (m, 5H), 8.09 (s, 2H), 8.67 (s, 1H); ¹³C NMR (CDCl₃) 21.6, 52.3, 113.60, 117.88, 121.57, 122.16, 123.04, 123.63, 124.69, 124.75, 124.88, 126.06, 126.56, 127.05, 128.49, 130.22, 134.15, 134.77, 137.88, 138.97, 140.30, 145.75, 167.00.

Hydrolysis and De-tosylation of 54 and 56.

To a solution of 54 (0.336 mmol) or 56 (0.302 mmol) in MeOH/H₂O (7:1.25) was added KOH (7 eq.) (Scheme 10). The reaction mixture was stirred at 80° C. overnight. The cooled solution was concentrated under reduced pressure and diluted with H₂O (10 mL). The pH of the solution was adjusted to ˜2-3, resulting to the precipitation of the product. Finally, the indoles 57 and 58 were collected by vacuum filtration.

### 3-(Benzofuran-2-yl)-indole-5-carboxylic Acid (57)

Yellow solid (70.1 mg, 75.1%). ¹H NMR (CD₃OD): 7.12 (s, 1H), 7.17-7.25 (m, 2H), 7.47-7.53 (m, 2H), 7.57 (d, 1H, J=6.9), 7.88 (s, 1H), 7.93 (dd, 2H, J=1.0, 8.6), 8.79 (s, 1H); ¹³C NMR (CD₃OD): 98.94, 107.80, 110.02, 111.17, 119.86, 122.43, 122.67, 122.81, 122.95, 123.51, 124.00, 124.87, 130.04, 139.59, 152.80, 154.01, 170.90.

### 3-(Benzothiophene-2-yl)-indole-5-carboxylic Acid (58)

Light beige solid (63 mg, 72%). ¹H NMR (CD₃OD): 7.25-7.37 (m, 2H), 7.50(d, 1H, J=8.6) 7.58 (s, 1H), 7.73 (s, 1H), 7.78-7.84 (m, 2H), 7.92 (dd, 1H, J=1.4, 8.6), 8.793 (s, 1H). ¹³C NMR (CD₃OD): 112.76, 113.49, 119.44, 123.0, 123.82, 123.98, 124.08, 124.84, 124.95, 125.61, 126.36, 126.54, 139.25, 139.82, 141.34, 142.54, 171.38.

### (ii) Synthesis of 6-benzofuran-2-yl-naphthalen-2-ol (64), 6-benzothiophen-2-yl-naphthalen-2-ol (65), 6-(indol-3-yl]-naphthalen-2-ol (67)

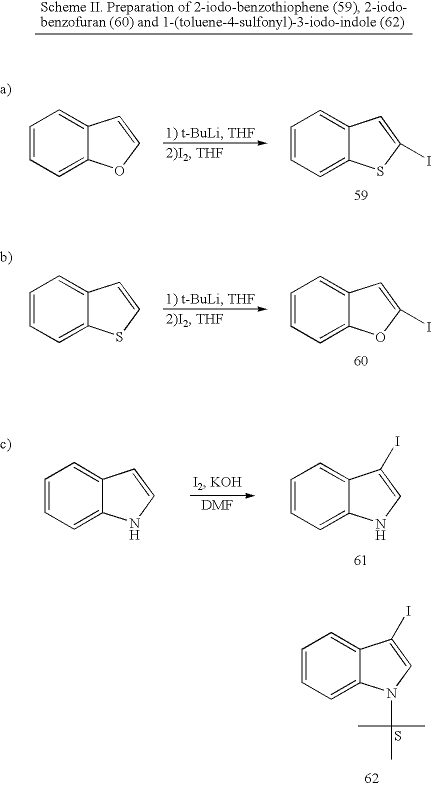

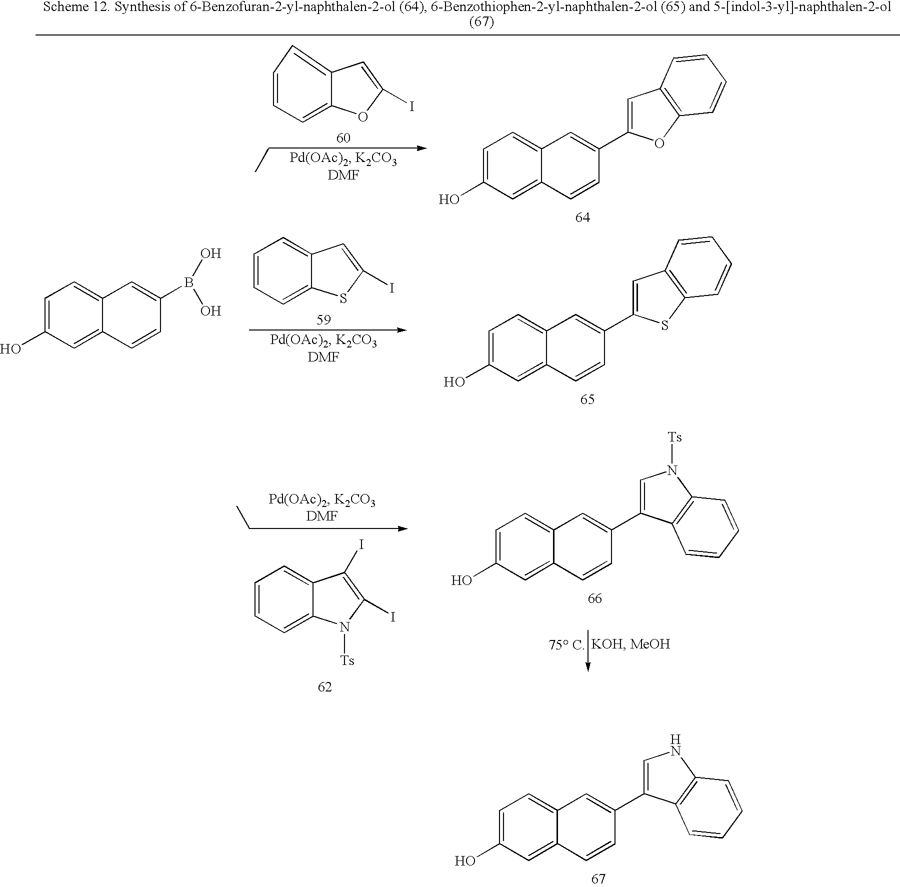

### 2-iodo-benzothoiphene (59)

To a solution of benzothiophene (1.0 g, 7.45 mmol) in THF (15 mL) was added t-BuLi (1.7 M, 4.8 mL, 1.1 eq.) at −78° C. (Scheme 11). After stirring at the same temperature for 30 minutes, a solution of I₂ (2.08 g, 1.1 eq.) in THF (5 mL) was added dropwise. The mixture was warmed up gradually to room temperature and stirred for 2 hours. The reaction was diluted with ethyl acetate (20 mL). Excess 12 was reduced with 10% Na₂S₂O₇ solution. The organic layer was washed with brine (2×10 mL), dried (MgSO₄) and concentrated under reduced pressure. Purification by flash column chromatography using hexane afforded 59 (1.60 g, 82%) as white solid. ¹H NMR (CDCl₃): 7.21-7.30 (m, 2H), 7.49 (s, 1H), 7.7 (m, 2H).

### 2-iodo-benzofuran (60)

To a solution of benzofuran (466 μL, 4.2 mmol) in THF (15 mL) was added t-BuLi (1.7 M, 2.73 mL, 1.1 eq.) at −78° C. (Scheme 11). After stirring at the same temperature for 30 minutes, a solution of I₂ (1.180 g, 1.1 eq.) in THF (5 mL) was added dropwise. The mixture was warmed up gradually to room temperature and stirred for 1.5 hours. The reaction was diluted with ethyl acetate (20 mL). Excess 12 was reduced with 10% Na₂S₂O₇ solution. The organic layer was washed with brine (2×10 mL), dried (MgSO₄) and concentrated under reduced pressure. Purification by flash column chromatography afforded 60 (0.826 g, 80%) as yellow-orange liquid. ¹H NMR (CDCl₃): 6.96 (s, 1H), 7.18-7.24 (m, 2H), 7.46-7.52) (m, 2H).

### 3-Iodo-indole (61)

To a mixture of indole (1.0 g, 8.5 mmol) and KOH (1.192 g, 2.5 eq.) in DMF (15 mL) was added I₂ (2.38 g, 9.4 mmol) (Scheme 11). After stirring at room temperature for 1 hour, the reaction mixture was poured to an ice-cold solution of 0.1% Na₂SO₃ (100 mL), resulting to the precipitation of the product. The product was filtered, dried under high vacuum pressure and used without further purification. Light orange solid (1.64 g, 80%). ¹H NMR (CDCl₃): 7.19-7.28 (m, 2H), 7.29 (d, 1H, J=2.5), 7.371 (d, 1H, J=7.9), 7.47 (d, 1H, J=7.8).

### 1-(Toluene-4-sulfonyl)-3-iodo-indole (62)

To a mixture of 3-Iodo-indole (0.500 g, 2.05 mmol) and NaOH (0.090 g, 1.1 eq.) in CH₂Cl₂ (10 mL) was added p-toluenesulfonylchloride (0.431 g, 1.1 eq.) (Scheme 11). After stirring at room temperature for 4 hours, the reaction was quenched with a saturated solution of NH₄Cl (10 mL). Layers were separated. The aqueous layer was extracted with CH₂Cl₂ (3×8 mL) and the combined organic layer was dried (MgSO₄), filtered and concentrated under reduced pressure to give 62 as white solid (0.680 g, 83%). Note that ˜10% of 3-Iodo-indole was recovered. ¹H NMR (CDCl₃): 2.35 (s, 3H), 7.21-7.40 (m, 5H), 7.70 (s, 1H), 7.78 (d, 2H, J=8.4), 7.96 (d, 1H, J=8.7).

### Suzuki Coupling of 6-hydoxy-2-naphthaleneboronic Acid (63) with 59, 60 and 62

To a degassed solution of 59, 60 or 62 (0.300-0.400 mmol) in DMF (4 mL) was added Pd(OAc)₂ (0.05 eq.), K₂CO₃ (2 eq.) and 6-hydroxy-3-naphthaleneboronic acid 63 (1.3 eq.) at room temperature (Scheme 12). After degassing and purging with argon (repeated thrice), the reaction mixture was stirred at 90° C. for 2-2.5 hours. The mixture was allowed to cool to room temperature and diluted with H₂O (15 mL). The aqueous solution was extracted with ethyl acetate (5×15 mL) and combined organic layer was concentrated under reduced pressure. The residue was purified by flash column chromatography using 30% ethyl acetate in hexane as solvent system to afford 64, 65 or 66.

### 6-Benzofuran-2-yl-naphthalen-2-ol (64)

Light brown solid (0.049 g, 46%); ¹H NMR (CDCl₃): 7.09 (s, 1H), 7.13-7.17 (m, 2H), 7.21-7.25 (m, 3H), 7.55 (d, 1H, J=7.2), 7.59 (d, 1H, J=6.7) 7.74 (d, 1H, J=7.7), 7.84 (d, 1H, J=7.6), 7.88 (d, 1H, J=8.1), 8.31 (s, 1H).

### 6-Benzothiophen-2-yl-naphthalen-2-ol (65)

White solid (0.065 g, 61%); ¹H NMR (DMSO): 7.12-7.20 (m, 2H), 7.36 (t, 1H, J=6.9), 7.40 (t, 1H, J=7.0), 7.77-7.93 (m, 5H), 7.98 (d, 1H, J=7.9), 8.16 (s, 1H); ¹³C NMR (DMSO): 109.30, 119.94, 119.96, 122.88, 124.04, 124.70, 124.94, 125.25, 127.45, 128.17, 128.34, 130.36, 134.96, 138.89, 141.14, 144.27, 156.58.

### 6-[1-Toluene-4-sulfonyl)-indol-3-yl]-naphthalen-2-ol (66)

White solid (0.056 g, 53%); ¹H NMR (CDCl₃): 2.33 (s, 3H), 7.14-7.23 (m, 4H), 7.29 (t, 1H, J=7.6), 7.39 (t, 1H, J=7.5), 7.63 (dd, 1H, J=1.5, 8.4), 7.72-7.87 (m, 6H), 7.98 (s, 1H), 8.07 (s, 1H), 8.07 (d, 1H, J=8.3).

### 6-[indol-3-yl]-naphthalen-2-ol (67)

To a solution of 66 (0.045 g, 0.105 mmol) in MeOH (0.500 mL) was added KOH (0.018 g, 3 eq.) at room temperature (Scheme 12). The reaction mixture was heated at 75° C. for 12 hours. The solvent was evaporated under reduced pressure. Water (7 mL) was added. The aqueous solution was extracted with ethyl acetate (3×5 ml). The combined organic layer was washed with a saturated solution of NH₄Cl solution (2×4 ml), dried (MgSO4), filtered and concentrated under reduced pressure. The residue was purified by flash column chromatography using 30% ethyl acetate in hexane as solvent system to afford 67 (0.20 g, 71.3%) as light yellow solid. ¹H NMR (acetone-d₆): 7.13-7.27 (m, 4H), 7.52 (d, 1H, J=8.0), 7.69-7.88 (m, 4H), 8.06 (d, 1H, J=7.9), 8.14 (s, 1H), 8.55 (s, 1H); ¹³CNMR: 108.92, 111.77, 117.11, 118.48, 119.45, 119.68, 121.69, 122.76, 124.50, 125.86, 126.52, 126.65, 129.13, 129.30, 130.91, 133.43, 137.41, 154.86. 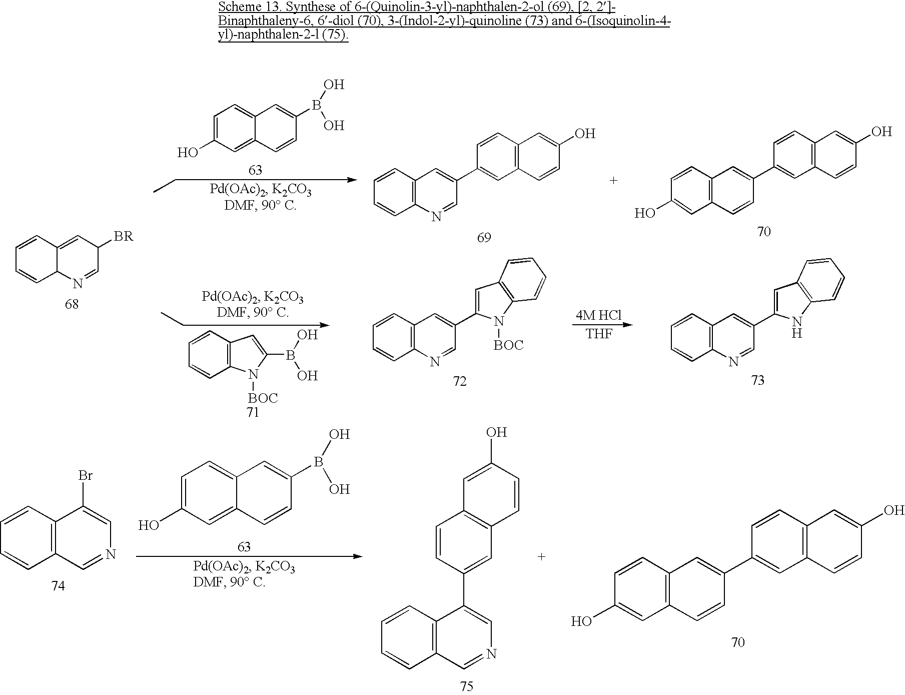
General Procedure for Formation of Aromatic-Aromatic Bond: Suzuki Coupling.

To a degassed solution of the aryl bromide (68, 74 or 77, Schemes 8 and 9) in DMF (4.0 mL) was added aryl boronic acid (53, 55, 63 or 71, 1.2 equiv), Pd(OAc)₂ (0.05 equiv) and K₂CO₃ (2 equiv) at room temperature. After degassing and purging with argon (repeated thrice), the reaction mixture was stirred at 90° C. Reaction times vary from 1.5 hours to 12 hours. The mixture was allowed to cool to room temperature and diluted with H₂O (15 mL). The aqueous solution was extracted with ethyl acetate (5×15 mL) and the combined organic layer was concentrated under reduced pressure. The residue was purified by flash column chromatography.

### 6-(Quinolin-3-yl)-naphthalen-2-ol (69)

Light yellow solid (0.107 g, 74%). ¹H NMR (DMSO): 7.18 (d, 1H, J=8.7), 7.21 (s, 1H), 7.67 (t, 1H, J=7.3), 7.78 (t, 1H, J=7.5), 7.86-7.95 (m, 3H), 8.07 (s, 1H), 8.36 (s, 1H), 8.74 (s, 1H), 9.39 (s, 1H), 9.90 (s, 1H); ¹³C NMR (DMSO): 109.1, 119.8, 125.7, 126.4, 127.5, 127.6, 128.3, 128.5, 128.8, 129.2, 129.8, 130.5, 131.6, 132.8, 133.4, 134.7, 147.1, 150.2, 156.5.

### [2,2′]-Binaphthalenyl-6,6′-diol (70)

White solid (0.013 g, 9% as side product). ¹H NMR (MeOD): 7.07-7.15 (m, 2H), 7.73 (d, 1H, J=9.16), 7.77-7.82 (m, 2H), 8.06 (s, 1H); ¹³C NMR (MeOD): 108.4, 118.3, 124.9, 125.4, 126.4, 128.9, 129.4, 134.1, 136.6, 155.2.

### 3-(Indol-2-yl)-quinoline (73)

Yellow solid (0.093 g, 56%). ¹H NMR (DMSO): 7.06 (t, 1H, J=7.4), 7.18 (t, 1H, J=7.4), 7.23 (s, 1H), 7.48 (d, 1H, J=8.1), 7.62 (d, 1H, J=7.8), 7.67 (t, 1H, J=7.3), 7.77 (t, 1H, J=7.1), 8.01 (d, 1H, J=8.0), 8.05 (d, 1H, J=8.0), 8.74 (s, 1H), 9.47 (s, 1H), 11.83 (s, 1H); ¹³C NMR (DMSO): 101.0, 111.9, 120.2, 120.8, 122.8, 126.0, 127.8, 128.1, 128.6, 129.0, 129.3, 129.8, 130.3, 135.2, 138.0, 147.1, 149.0.

### 6-(Isoquinolin-4-yl)-naphthalen-2-ol (75)

White solid (0.098 g, 75%). ¹H NMR (DMSO): 7.18 (dd, 1H, J=2.2, 8.8), 7.24 (d, 1H, J=2.0, 7.57 (d, 1H, J=8.4), 7.75 (t, 1H, J=7.7), 7.80 (t, 1H, J=7.7), 7.87-7.90 (m, 2H), 7.93 (d, 1H, J=8.4), 8.53 (s, 1H), 9.37 (s, 1H), 9.9 (s, 1H); ¹³C NMR (DMSO): 109.1, 119.7, 124.7, 126.8, 127.9, 128.2, 128.6, 129.1, 130.2, 131.1, 131.5, 133.2, 133.9, 134.6, 143.2, 152.2, 156.4.

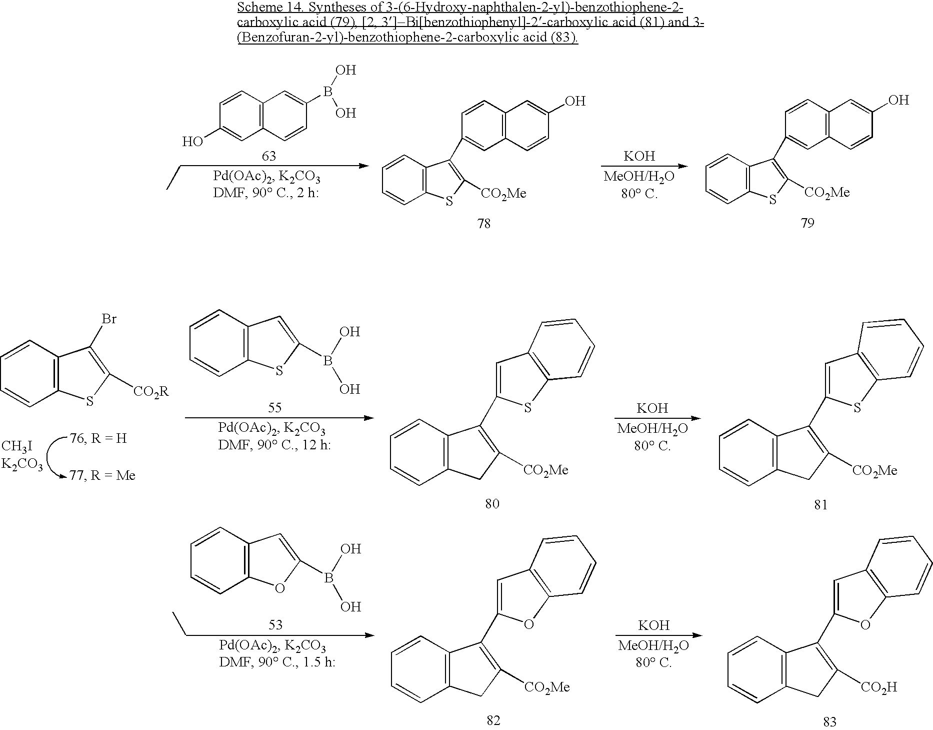

### 3-(6-Hydroxy-naphthalen-2-yl)-benzothiophene-2-carboxylic Acid Methyl Ester (78)

White solid (0.097 g, 78%). ¹H NMR (DMSO): 3.71 (s, 3H), 7.15 (dd, 1H, J=2.3, J=8.8), 7.23 (d, 1H, J=2.0), 7.39-7.48 (m, 2H), 7.56 (d, 1H, J=8.1), 7.60 (t, 1H, J=7.5), 7.80 (d, 1H, J=8.5), 7.82-7.87 (m, 2H), 8.14 (d, 1H, J=8.1); ¹³C NMR (DMSO): 52.8, 109.1, 119.4, 123.5, 125.5, 125.9, 126.1, 127.7, 127.8, 128.1, 128.5 (2C), 128.9, 130.2, 136.5, 140.0 (2C), 144.2, 156.4, 162.7.

### 3-(6-Hydroxy-naphthalen-2-yl)-benzothiophene-2-carboxylic Acid (79)

White solid (0.085 g, 98%). ¹H NMR (DMSO): 7.14 (dd, 1H, J=2.3, J=8.8), 7.21 (d, 1H, J=2.1), 7.39-7.46 (m, 2H), 7.51 (d, 1H, J=8.0), 7.56 (t, 1H, J=7.0), 7.78(d, 1H, J=8.5), 7.83 (s, 1H), 7.84 (d, 1H, J=8.8), 8.11 (d, 1H, J=8.1), 9.83 (s, 1H), 13.15 (s, 1H). ¹³C NMR (DMSO); 109.1, 119.4, 123.4, 125.3, 125.7, 126.0, 127.7, 127.8, 128.7, 128.8, 128.9, 130.0, 130.2, 134.6, 139.9, 140.4, 143.2, 156.3, 163.9; HRMS: C₁₉H₁₂O₃S calculated 320.0507, found 320.0501.

### [2,3′]-Bi[benzothiophenyl]-2′-carboxylic Acid Methyl Ester (80)

White solid (0.082 g, 72%). ¹H NMR (DMSO): 3.83 (s, 3H), 7.35-7.43 (m, 4H), 7.51 (t, 1H, J=7.6), 7.79 (d, 1H, J=7.4), 7.83-7.92 (m, 3H); ¹³C NMR (DMSO): 52.5, 122.2, 122.5, 123.9, 124.5, 125.2, 125.2, 125.4, 127.5, 130.5, 134.8, 135.8, 139.7, 140.06, 140.1, 140.9, 162.5.

### [2,3′]-Bi[benzothiophenyl]-2′-carboxylic Acid (81)

White solid (0.065 g, 91%). ¹H NMR (DMSO): 7.39-7.51 (m, 3H), 7.53-7.62 (m, 2H), 7.7 (d, 1H, J=8.0), 7.94 (d, 1H, J=7.2), 8.04 (d, 1H, J=7.4), 8.13 (d, 1H, J=8.1), 13.50 (s, 1H); ¹³C NMR (DMSO): 122.7, 123.5, 124.4, 124.9, 125.0, 125.1, 126.0, 126.1, 128.0, 133.0, 134.3, 134.8, 139.5, 139.9, 140.0, 140.5, 163.3; HRMS: C₁₇H₁₀O₂S₂ calculated 310.0122, found 310.0122.

### 3-(Benzofuran-2-yl)-benzothiophene-2-carboxylic Acid Methyl Ester (82)

White solid (0.105 g, 92%). ¹H NMR (DMSO): 3.89 (s, 3H), 7.24 (s, 1H), 7.30 (t, 1H, J=7.4), 7.36 (t, 1H, J-7.1), 7.5(t, 1H, J=7.1), 7.6(d, 1H, J=8.2), 7.7 (d, 1H, J=7.7), 7.89 (d, 1H, J=8.1), 8.10 (d, 1H, J=8.2); ¹³C NMR (DMSO): 52.6, 108.8, 111.3, 121.5, 122.5, 123.0, 124.9, 125.4, 125.6, 127.4, 128.5, 130.7, 131.5, 138.8, 140.2, 148.9, 155.0, 162.5.

### 3-(Benzofuran-2-yl)-benzothiophene-2-carboxylic Acid (83)

White solid (0.094 g, 99%). ¹H NMR (DMSO): 7.31-7.43 (m, 3H), 7.54 (t, 1H, J=7.9), 7.61 (t, 1H, J=7.1), 7.68 (d, 1H, J=8.7), 7.78 (d, 1H, J=7.3), 7.99 (d, 1H, J=7.5), 8.15 (d, 1H, J=8.4), 13.62 (bs, 1H); ¹³C NMR (DMSO): 108.7, 111.7, 122.0, 123.59, 123.6, 125.2, 125.4, 126.3, 128.0, 128.7, 130.0, 138.8, 139.6, 148.9, 154.9, 163.3; HRMS: C₁₇H₁₀O₃S calculated 294.0350, found 294.0350.

### EXAMPLE 7

### Stille Coupling Synthesis of Bis-indoles

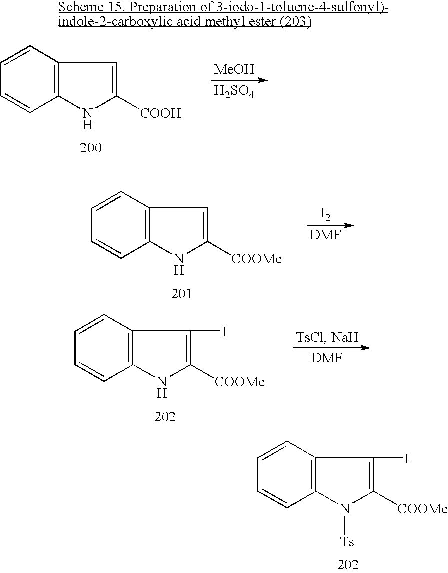

### Indole-2-carboxylic Acid Methyl Ester (201)

Commercially available indole-2-carboxylic acid 200 (1.61 g, 10 mmol) and concentrated H₂SO₄ (0.5 mL) were refluxed in dry MeOH (50 mL) for 12 hours. The solution was then cooled to room temperature and concentrated under reduced pressure. Water (25 mL) was added to the residue and adjusted the pH to 7. The aqueous layer was extracted with ethyl acetate (3×15 mL). The combined organic phase was dried with MgSO₄, filtered and concentrated under vacuum to afford 201 (1.75 g 100%). The product was used for the next step without further purification.

### 3-iodo-indole-2-carboxylic Acid Methyl Ester (202)

To a solution of 201 (0.885 g, 5 mmol) and KOH (0.713 g, 12.5 mmol) in DMF (15 mL) was added I₂ (1.396 g, 5.5 mmol) at room temperature. After stirring for 1.5 hours, the reaction mixture was poured to an ice-cold solution of Na₂SO₃ (0.1%), the precipitation was filtered and dried. The product (1.46 g, 90%) was used for the next step without further purification.

### 3-iodo-1-(toluene-4-sulfonyl)-indole-2-carboxylic Acid Methyl Ester (203)

To a mixture of 202 (0.604 g, 2 mmol) and NaH (60%, 0.192 g, 2.4 mmol) in DMF (20 mL) was added p-toluenesulfonylchloride (0.381 g, 2 mmol) at room temperature. After stirring for 1 hour, ethyl acetate (50 mL) was added to the reaction mixture. The mixture was washed with brine (3×30 mL). The organic layer was dried with MgSO₄, filtered and concentrated under vacuum. The residue was purified by flash chromatography (10% ethyl acetate/hexane V:V) to afford 203 (0.68 g, 76%). ¹H NMR (CDCl₃): 2.35 (s, 3H), 4.05 (s, 3H), 7.23 (d, 2H, J=8.4), 7.33 (t, 1H, J=7.2), 7.41, (m. 2H), 7.83 (d, 2H, J=8.4), 7.98 (d, 1H, J=8.4).

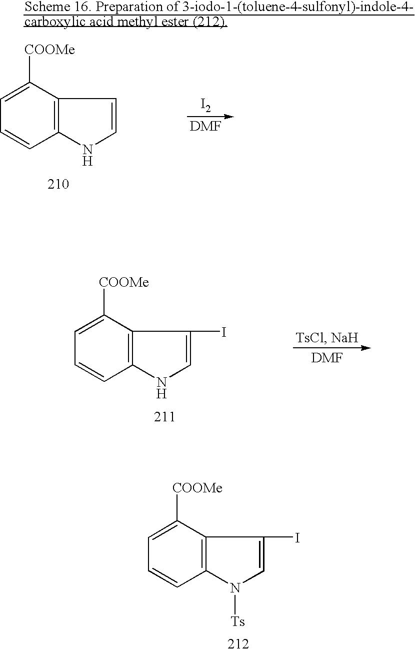

### 3-iodo-indole-4-carboxylic Acid Methyl Ester (211)

To a solution of commercially available 210 (0.525 g, 3 mmol) and KOH (0.428 g, 7.5 mmol) in DMF (15 mL) was added I₂ (0.838 g, 3.3 mmol) at room temperature. After stirring for 1.5 hours, the reaction mixture was poured on an ice-cold solution of Na₂SO₃ (aq., 0.1%). The aqueous layer was extracted with ethyl acetate (3×15 mL). The combined organic layers were dried with MgSO₄, filtered and concentrated under vacuum. The residue was purified by flash chromatography (3% ethyl acetate/hexane V:V) to afford 211 (0.758 g, 84%). ¹H NMR (CDCl₃): 4.03 (s, 3H), 7.24 (d, 1H, J=7.8), 7.43 (d, 1H, J=2.5), 7.52 (t, 2H, J=8.5), 8.63 (s, 1H).

### 3-iodo-1-(toluene-4-sulfonyl)-indole-4-carboxylic Acid Methyl Ester (212)

To a mixture of 211 (0.604 g, 2 mmol) and NaH (60%, 0.192 g, 2.4 mmol) in DMF (20 mL) was added p-toluenesulfonylchloride (0.381 g, 2 mmol) at room temperature. After stirring for 1 hour, ethyl acetate (50 mL) was added to the reaction mixture. The mixture was washed with brine (3×30 mL). The organic layer was dried with MgSO₄, filtered and concentrated under vacuum. The residue was purified by flash chromatography (10% ethyl acetate/hexane V:V) to afford 212 (0.65 g, 71%).

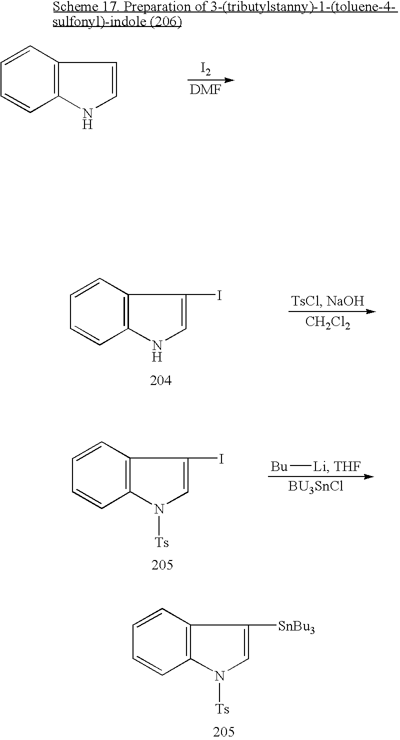

### 3-iodo-indole (204)

To a solution of indole (1.0 g, 8.5 mmol) and KOH (1.192 g, 21.25 mmol) in DMF (15 mL) was added 12 (2.38 g, 9.4 mmol) at room temperature. After stirring for 1 hour, the reaction mixture was poured on an ice-cold solution of Na₂SO₃ (aq., 0.1%), and the resulting precipitate was filtered and dried. The product (1.83 g, 88%) was used without further purification.

### 3-iodo-1-(toluene-4-sulfonyl)-indole (205)

To a mixture of 204 (0.5 g, 2.05 mmol) and NaOH (0.09 g, 2.3 mmol) in CH₂Cl₂ (10 mL) was added p-toluenesulfonylchloride (0.431 g, 2.3 mmol) at room temperature. After stirring for 4 h, the reaction was quenched with a saturated solution of NH₄Cl (aq.) (10 mL). Layers were separated, and the aqueous layer was extracted with CH₂Cl₂ (3×8 mL). The combined organic layers were dried with MgSO₄, filtered and concentrated under vacuum. The residue was purified by flash chromatography (3% ethyl acetate/hexane V:V) to afford 205 (0.68 g, 83%). ¹H NMR (CDCl₃): 2.35 (s, 3H), 2.21-7.40 (m, 5H), 7.70 (s, 1H), 7.78 (d, 2H, J=8.4), 7.96 (d, 1H, J=8.7).

### 3-(tributylstannyl)-1-(toluene-4-sulfonyl)-indole (206)

To a solution of 205 (0.796 g, 2 mmol) in THF (20 mL) at −78° C. was added butyllithium (1.6 mL, 2.5 M solution, 4 mmol). The solution was stirred for 20 min, and tributyltin chloride (0.56 mL, 2.06 mmol) was added. The mixture was allowed to warm to room temperature and stirred for 4 h. A saturated aqueous solution of KF was poured into the reaction and stirred for 30 min. The aqueous phase was extracted with ethyl acetate (3×30 mL). The combined organic phase was dried with MgSO₄, and the solvent was removed in vacuo to afford 206. The product was used directly for the next step.

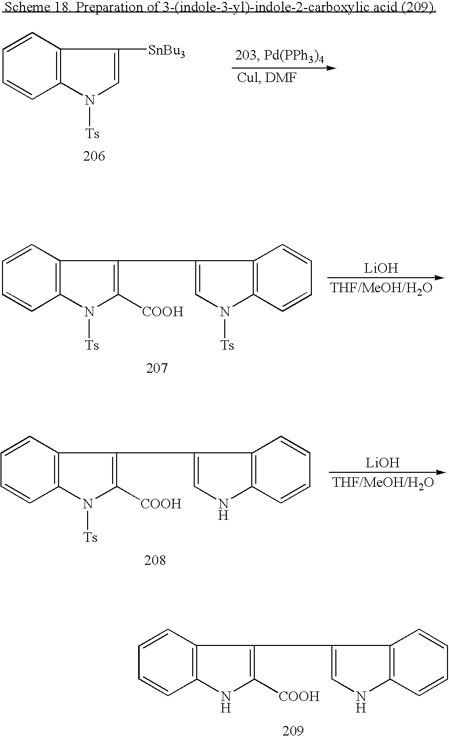

### 3-(1-(toluene-4-sulfonyl)-indol-3-yl)-1-(toluene-4-sulfonyl)-indole-2-carboxylic acid methyl ester (207)

A solution of 203 (0.279 g, 0.613 mmol), crude 206 (0.430 g, 0.766 mmol), a catalytic amount of CuI and tetrakis(triphenylphosphine)palladium in DMF (20 mL) was degassed with argon for 10 min. The mixture was brought to 90° C. and stirred for 8 h. The solvent was removed in vacuo and the residue was purified by flash chromatography (15% ethyl acetate/hexane V:V) to afford 207. ¹H NMR (CDCl₃): 2.36 (s, 3H), 2.37 (s, 3H), 3.38, (s, 3H), 7.20 (m, 1H), 7.23-7.27 (m, 4H), 7.32 (d, 2H, J=7.6), 7.35 (d, 2H, J=7.6), 7.43 (m, 1H), 7.78 (s, 1H), 7.83 (d, 2H, J=8.4), 7.89 (d, 2H, J=8.4), 8.04 (d, 1H, J=8.4), 8.11 (d, 1H, J=8.4).

### 3-(indol-3-yl)-1-(toluene-4-sulfonyl)-indole-2-carboxylic Acid (208)

To a solution of 207 (0.2 g, 0.33 mmol) in THF/MeOH/H₂O (5:5:1) was added LiOH (0.052 g, 2.2 mmol). The reaction mixture was stirred and refluxed. After 5 h, the solution was cooled and concentrated. Water (10 mL) was added and pH was adjusted to 2 with 1N HCl. The aqueous phase was extracted with ethyl acetate (3×10 mL). The combined organic phase was dried with MgSO₄, and the solvent was removed in vacuo. The residue was purified by flash chromatography (ethyl acetate/hexane/acetic acid, 30:70:1, V:V) to afford 208 (92 mg, 64%). ¹H NMR (DMSO): 2.34 (s, 3H), 7.07 (t, 1H, J=7.5), 7.22 (t, 1H, J=7.5), 7.26 (d, 1H, J=7.7), 7.30-7.32 (m, 2H), 7.36 (t, 1H, J=7.6), 7.42 (d, 2H, J=8.2), 7.53 (d, 1H, J=8.5), 7.89 (s, 1H), 7.93, (d, 2H, J=8.2), 7.99 (d, 1H, J=8.3), 11.99 (s, 1H), 12.97 (s, 1H); ¹³C NMR (DMSO): 21.5, 111.8, 113.2, 113.7, 116.1, 120.7, 121.2, 121.6, 123.7, 125.0, 125.3, 125.7, 126.2, 127.3, 127.7, 130.7, 131.0, 134.6, 134.7, 136.6, 145.9, 163.1.

### 3-(indole-3-yl)-indole-2-carboxylic Acid (209)

To a solution of 208 (60 mg, 0.14 mmol) in THF/MeOH/H₂O (2:5:1) was added LiOH (48 mg, 2 mmol). The reaction mixture was refluxed for 30 h. The solution was cooled and concentrated. Water (10 mL) was added and pH was adjusted to 2. The aqueous phase was extracted with ethyl acetate (3×5 mL). The combined organic phase was dried with MgSO₄, and the solvent was removed in vacuo. The residue was purified by flash chromatography (ethyl acetate/hexane/acetic acid, 30:70:1, V:V) to afford 209 (30 mg, 78%). ¹H NMR (DMSO): 6.97 (t, 1H, J=7.4), 7.02 (t, 1H, J=7.4), 7.11 (t, 1H, J=7.4), 7.25-7.29 (m, 2H), 7.44-7.50 (m, 4H), 11.27 (s, 1H), 11.68 (s, 1H), 12.68 (s, 1H); ¹³C NMR (DMSO): 107.5, 111.6, 112.4, 115.5, 118.5, 119.5, 119.9, 120.8, 121.5, 123.9, 124.5, 125.4, 127.0, 127.7, 136.0, 136.2, 163.1.

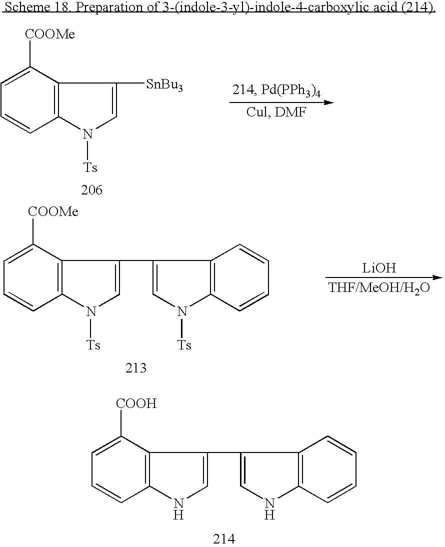

### 3-(1-(toluene-4-sulfonyl)-indol-3-yl)-1-(toluene-4-sulfonyl)-indole-4-carboxylic Acid Methyl Ester (213)

A solution of 212 (0.230 g, 0.505 mmol), crude 206 (0.360 g, 0.641 mmol), catalytic amount of CuI and tetrakis(triphenylphosphine)palladium in DMF (20 mL) was degassed with argon for 10 min. The mixture was brought to 90° C. and stirred for 8 h. The solvent was removed in vacuo and the residue was purified by flash chromatography (15% ethyl acetate/hexane V:V) to afford 213 (210 mg, 70%). ¹H NMR (CDCl₃): 2.36 (s, 3H), 2.38 (s, 3H), 2.43 (s, 3H), 7.14-7.18 (m, 2H), 7.26-7.29 (m, 4H), 7.30-7.34 (m, 1H), 7.41 (t, 1H, J=8.0), 7.58 (s, 1H), 7.65 (d, 1H, J=7.5), 7.73 (s, 1H), 7.82 (d, 2H, J=8.3), 7.87 (d, 2H, J=8.3), 8.05 (d, 1H, J=8.4), 8.25, (d, 1H, J=8.4), ¹³C NMR (CDCl₃): 21.6, 21.7, 50.5, 113.6, 114.1, 117.1, 117.3, 120.3, 122.9, 123.6, 124.5, 125.0, 125.6, 125.9, 127.0, 127.1, 127.2, 127.3, 130.0, 130.2, 131.1, 134.8, 134.9, 135.3, 136.0, 145.1, 145.6, 167.7.

### 3-(indol-3-yl)-indole-4-carboxylic Acid (214)

To a solution of 213 (200 mg, 0.334 mmol) in THF/MeOH/H₂O (2:5:1) was added LiOH (48 mg, 2 mmol). The reaction mixture was refluxed for 30 h. The solution was cooled and concentrated. Water (10 mL) was added and pH was adjusted to 2 with 1N HCl. The aqueous phase was extracted with ethyl acetate (3×5 mL). The combined organic phase was dried with MgSO₄, and the solvent was removed in vacuo. The residue was purified by flash chromatography (ethyl acetate/hexane/acetic acid, 30:70:1, V:V) to afford 214 (50 mg, 54%). ¹H NMR (CD₃OD): 6.93 (t, 1H, J=8.5), 7.06 (t, 1H, J=8.5), 7.17-7.21 (m, 2H), 7.35 (d, 1H, J=8.2), 7.39-7.42 (m, 3H), 7.61 (d, 1H, J=8.2) 10.30 (s, 1H); ¹³C NMR (CD₃OD): 110.3, 110.6, 111.4, 114.4, 118.2, 119.2, 120.0, 120.4, 120.6, 121.9, 124.0, 125.6, 125.7, 128.6, 136.4, 137.7, 172.0.

### EXAMPLE 8

### In vitro Analysis of Bi-Aromatic Compounds

Materials

Aβ¹⁻⁴⁰ (AnaSpec, San Jose, Calif., lots 14212, 34862 and 34889), Aβ¹⁻⁴² (AnaSpec, lot 45685) and α-synuclein (rPeptide, Bogart, GA, lot 121303AS) were stored at −80° C. until used. Positive and negative controls, Thioflavin T, Thioflavin S and materials for the tris buffers were obtained from Sigma (St. Louis, Mo.). Reagents and starting materials for chemical synthesis of bi-aromatics were obtained from Aldrich (St. Louis, Mo.), Alfa Aesar (Ward Hill, Mass.) and Combi-Blocks Inc. (San Diego, Calif.). All water used in the in vitro studies was micropore filtered and deionized.

The tau441 expression construct [L Buee et al. (2000). Brain Research Reviews 33:95-130] was purchased (Bioclon Inc., San Diego, Calif.) already transformed in BL21 D3 bacterial cells (Novagen/EMD Biosciences) for protein expression. Cells were grown and induced with IPTG according to Novagen's protocol. To lyse cells, Cellytic B reagent (Sigma) was used with lysozyme protease inhibitors (Sigma) and benzonase (Novagen/EMD Biosciences), according to manufacturer's methods. Lysate was cleared by centrifugation for 20 min at 10,000 RPM (SS34 rotor, Sorvall RC2b centrifuge) at 4° C. Protein was purified from the soluble fraction. Once purified, tau441 was stored at −78° C. as frozen aliquots (8.3 mg/mL, 60 μL) in Tris-HCl (50 mM, pH 7.4) until used.

Stock Solutions of Aβ¹⁻⁴⁰, Aβ¹⁻⁴², tau441 and α-synuclein

Aβ¹⁻⁴⁰ (1.0 mg) was pre-treated in a 1.5 mL microfuge tube with 1,1,1,3,3,3-hexafluoroisopropanol (HFIP, 1 mL) and sonicated for 20 min. to disassemble any pre-formed Aβ aggregates. The HFIP was removed with a stream of argon and the Aβ dissolved in Tris base (5.8 mL, 20 mM, pH 10.5). The pH was adjusted to 7.4 with concentrated HCl and the solution filtered using a syringe filter (0.2 μm). This solution was used for circular dichroism studies or diluted with an equal volume of 8 μM Thioflavin T (ThT) in Tris-HCl (20 mM, pH 7.4, 300 mM NaCl) for the ThT aggregation assay.

Aβ¹⁻⁴² (1.0 mg) was either pretreated with HFIP in the same manner as Aβ¹⁻⁴⁰ or this step was omitted. After removing HFIP, when appropriate, Aβ¹⁻⁴² was dissolved in 1% NH₃ (aq.) (200 μL) and sonicated for 1 min. The solution was diluted with Tris-HCl (5.7 mL, 20 mM, pH 7.4), the pH adjusted to 7.4 with concentrated HCl (aq.) and the solution filtered using a syringe filter (0.2 μm). Prior to use in the ThT aggregation assay, the solution was diluted with an equal volume of 8 μM Thioflavin T (ThT) in Tris-HCl (20 mM, pH 7.4, 300 mM NaCl).

Frozen aliquots of tau441 (8.3 mg/mL, 60 μL) in Tris-HCl (50 mM, pH 7.4) were allowed to thaw at room temperature (RT) before being diluted with Tris-HCl (2.64 mL, 50 mM, pH 7.4) containing dithiothreitol (DTT, 1 mM) to prevent disulfide bonds and NaN₃ (50 μM) to prevent bacterial growth. After allowing to stand at RT for 1 h, Thioflavin S (ThS) was added (2.5 μL, 10.8 mM), followed by the aggregation inducer heparin (20 μL, 1.08 g/mL).

α-Synuclein (1.0 mg) was dissolved directly in Tris-HCl (11.53 mL, 20 mM, pH 7.4, 100 mM NaCl) containing DTT (5 mM) and ThT (10 μM).

### (i) Kinetic Thioflavin T/Thioflavin S Assay (LeVine, H., Protein Science, 2: 404-10 (1993))

Aliquots (200 μL) of Aβ¹⁻⁴⁰, Aβ¹⁻⁴², tau441 or α-synuclein with ThT or ThS were added to wells of a black polystyrene 96-well plate, followed by 2 μL of a compound in DMSO (various concentrations), or DMSO alone (controls). For α-synuclein, sodium dodecyl sulfate solution (2.0 μL, 30 mM) was also added to each well to induce aggregation. Incubations were performed in triplicate and were taken to contain 20 μM Aβ¹⁻⁴⁰ or Aβ¹⁻⁴², 6 μM tau441 or 4 μM α-synuclein. Plates were covered with clear polystyrene lids and incubated at 37° C. in a GENios microplate reader obtained from Tecan Group Limited, Mannedorf, Zurich. For Aβ¹⁻⁴⁰, tau441 and α-synuclein, fluorescence readings (λₑₓ=450 nm, λₑₘ=480 nm) were taken every 15 min., after first shaking at high intensity for 15 sec. and allowing to settle for 10 sec. before each reading. For Aβ¹⁻⁴², readings were taken every 5 min.

The absorbance of each compound was measured under the conditions used during testing, though on ten times the scale (2 mL), to ensure no significant quenching interfered with the assay. This step was also used to identify whether compounds were completely soluble under testing conditions. Results for those compounds found to absorb significantly (molar absorptivity coefficient at 450 nm greater than 500 cm⁻¹ M⁻¹) or to not fully dissolve were not used.

“Seeded” Thioflavin T Aggregation Assay for Aβ¹⁻⁴⁰

The same procedure as described in Thioflavin T Aggregation Assay was used for “seeded” ThT aggregarion assay for Aβ¹⁻⁴⁰ but the HFIP pretreatment was omitted. The presence of small Aβ¹⁻⁴⁰ aggregates in the untreated batches caused aggregation to begin immediately rather than after a 12-24 hr. lag period.

Aβ¹⁻⁴⁰ Disaggregation Assay

Aβ¹⁻⁴⁰ was allowed to aggregate under conditions described in Thioflavin T Aggregation Assay except that no DMSO was added initially. After 46 hrs., compound in DMSO or vehicle was added (2 μL) and the fluorescence measured every 15 min. as detailed above.

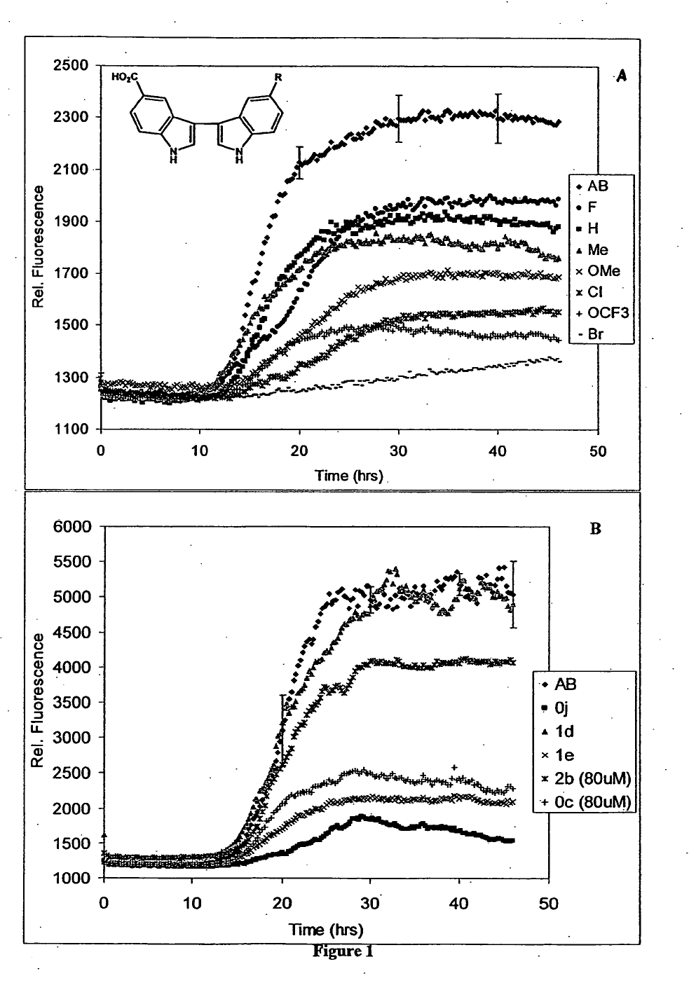

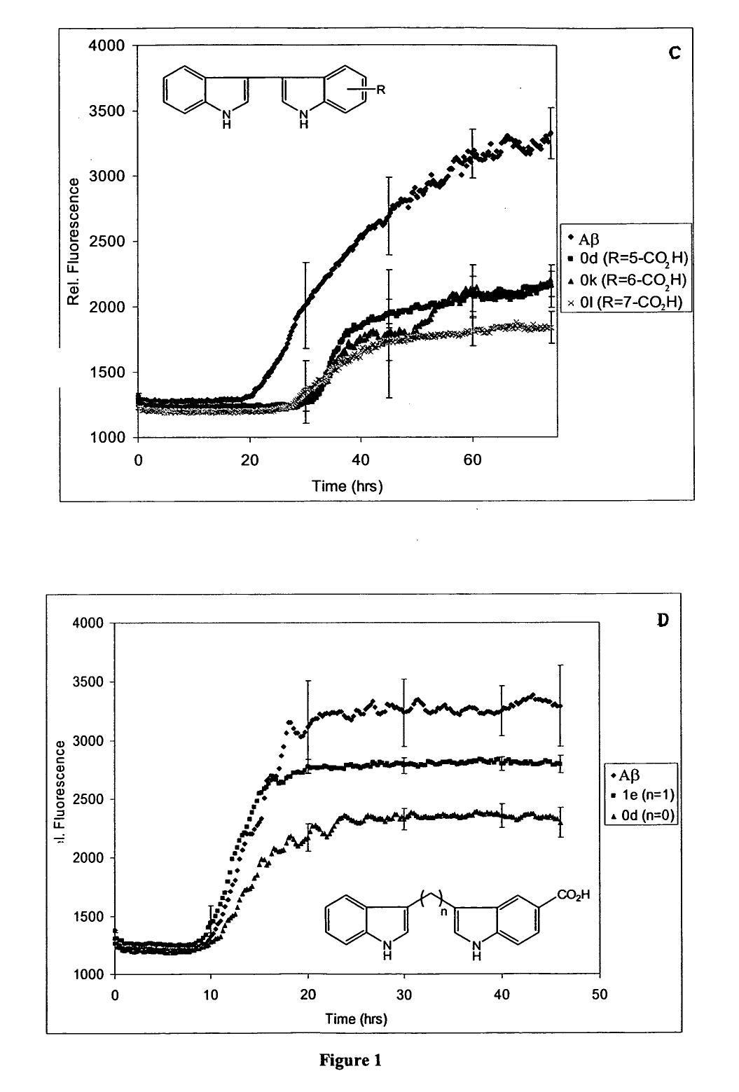

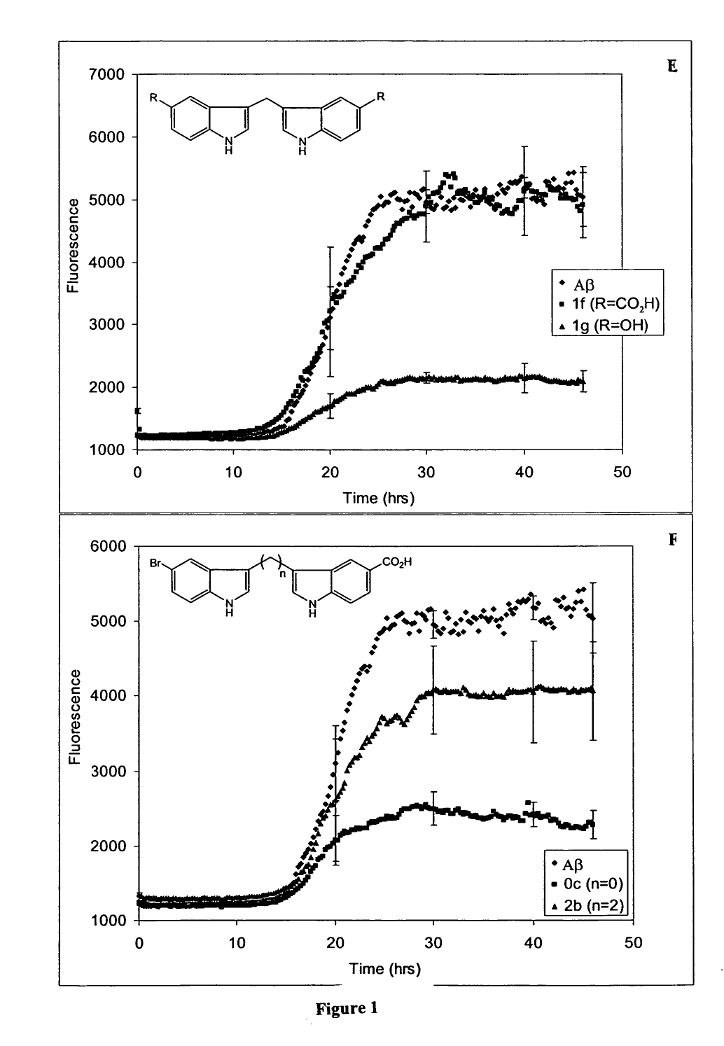

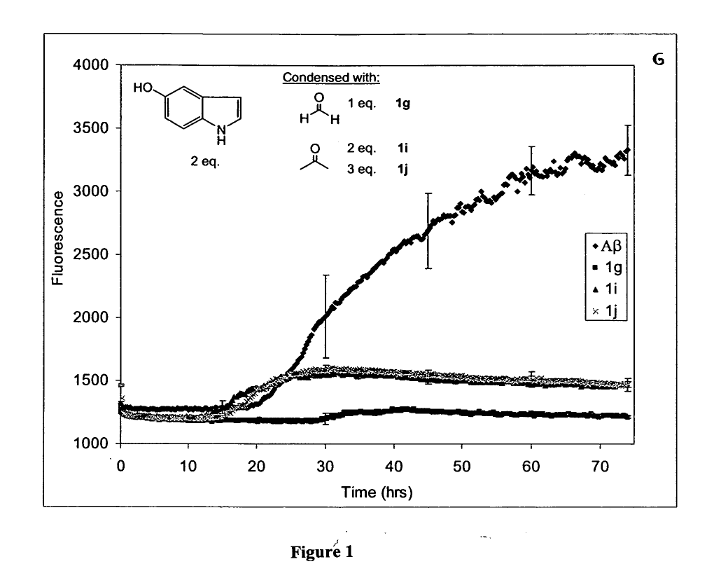

Several bis-indole compounds decreased Aβ¹⁻⁴⁰ and Aβ¹⁻⁴² aggregation, seen as a decrease in ThT fluorescence of at 480 nm (FIGS. 1-5, 7, 8). SDS was used as a positive control since it is known to stabilize the α-helical, monomeric form of Aβ¹⁻⁴⁰ at concentrations above its critical micelle concentration of 8 mM. It was present at 16 mM in the SDS incubation. The potent anti-amyloidogenic polyphenol compound morin (Ono, K et al. J. Neurochem, 2003, 87: 172-181) was also used as a positive control.

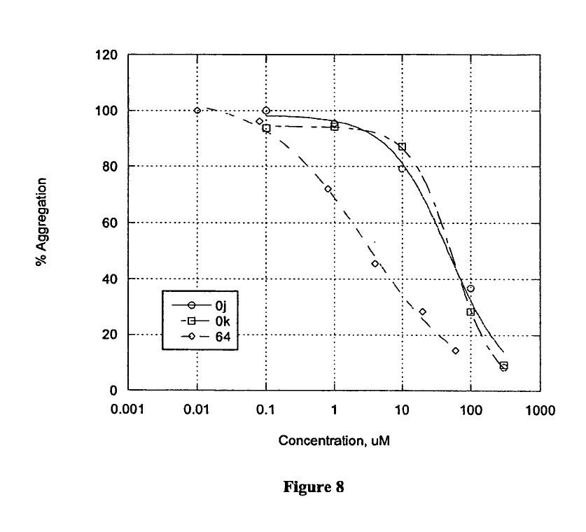

Bi-aromatic compounds other than bis-indoles were also found to inhibit the aggregation of Aβ¹⁻⁴⁰ and Aβ¹⁻⁴² (FIGS. 1-5, 7, 8). Aza-indole-containing compounds 10n, 0n and 0o, for example, as well as compounds containing one or no indoles were found to inhibit aggregation of both isoforms of the peptide (FIGS. 1C, D, E, F, 7, 8). For indole-phenol compounds, it was found that when indole was linked to two phenol groups (102), it was active at 200 μM, whereas when it was linked to a single phenol (103), it was not (FIG. 1D). Dose-response activity is shown for some compounds against Aβ¹⁻⁴⁰ (FIG. 3) and Aβ¹⁻⁴² (FIG. 8) aggregation. All compounds tested against Aβ¹⁻⁴⁰ and Aβ¹⁻⁴² were found to have similar activity against both.

FIG. 1 depicts the inhibition of Aβ¹⁻⁴⁰ aggregation, as measured by Thioflavin T (ThT) fluorescence, by directly-linked 3,3′-bis-indolyl compounds 0c-i (A), other bis-indoles (B), aza-indole-containing compounds (C), indole-phenols (D), indole-5-carboxylic acid-containing bi-aromatics (E), and naphthol-containing bi-aromatics (F). Aggregation conditions: 20 μM Aβ¹⁻⁴⁰ incubated in covered black 96-well polystyrene microplates with 4 μM ThT, pH 7.4, Tris-HCl (20 mM), 150 mM NaCl, 1% DMSO. Compound concentrations are 200 μM (A,B, except where otherwise noted), 100 μM (E), 20 μM (F), or as indicated (C,D). Plates heated at 37° C. in Tecan Genios microplate reader. All incubations performed in triplicate. Prior to experiment, Aβ¹⁻⁴⁰ pretreated with 1,1,1,3,3,3-hexafluoroisopropanol (HFIP). Fluorescence readings: fluorescence measured every 15 min. (λₑₓ=450 nm, λₑₘ=480 nm) after shaking for 15 sec. and pausing 10 sec. before taking measurement. Values are the mean of three replicates. Error bars, omitted for clarity in (B), represent standard deviation of the mean.

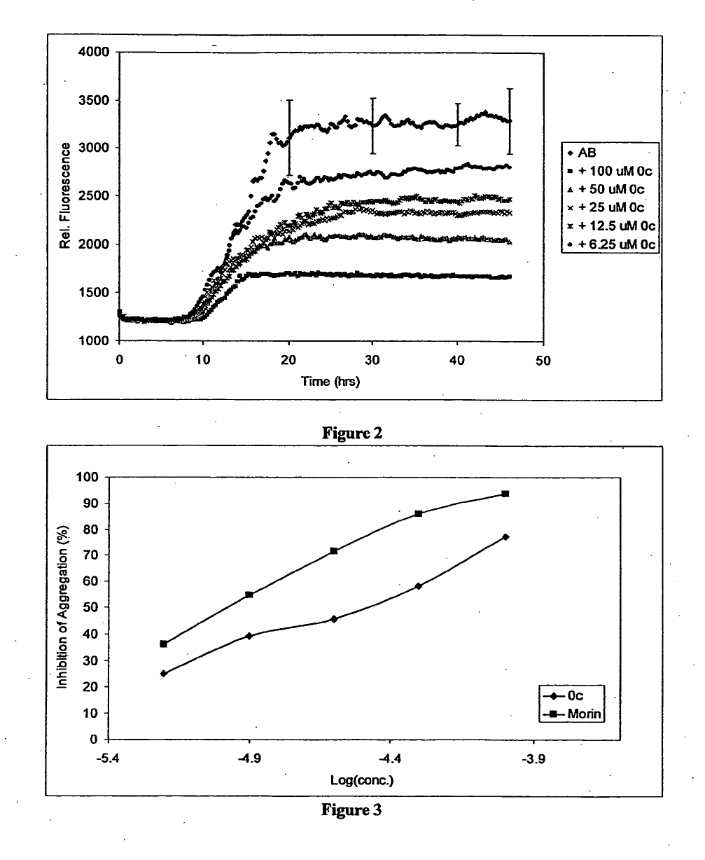

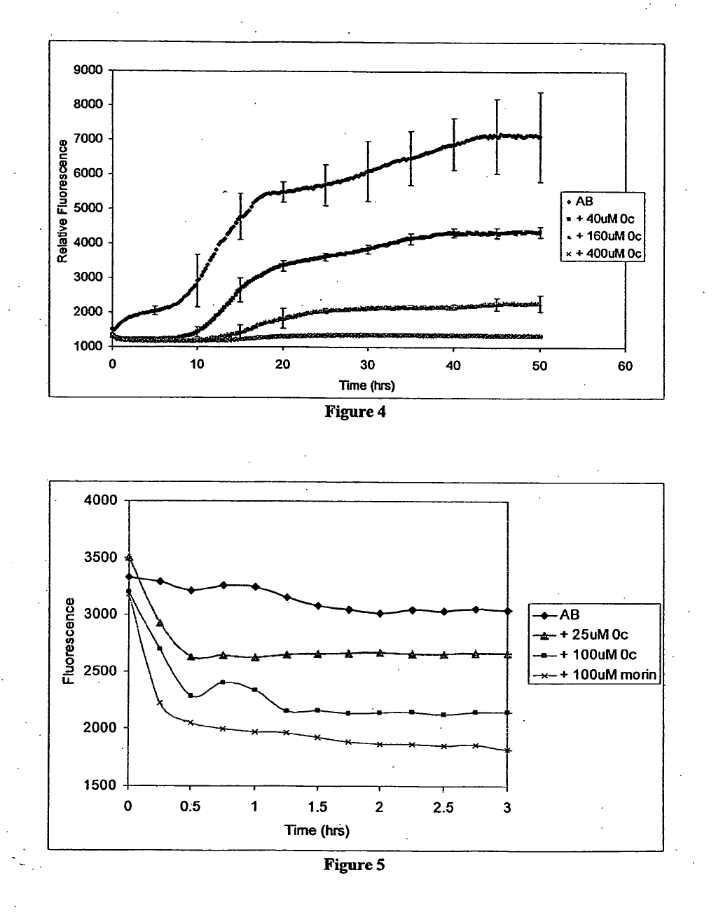

FIG. 2 depicts dose-response effect of 0c on Aβ¹⁻⁴⁰ aggegation in kinetic ThT assay. FIG. 3 depicts dose-response curves for inhibition of Aβ¹⁻⁴⁰ aggregation by 0j and positive control, morin. FIG. 4 depicts the inhibition of Aβ¹⁻⁴⁰ aggregation by 0c in “seeded” ThT assay. FIG. 5 depicts disaggregation of Aβ¹⁻⁴⁰ by 0c and positive control, morin. After incubating at 37° C. for 46 hrs. 0c, morin or vehicle were added as DMSO solutions (1:100) and the fluorescence measured every 15 min.

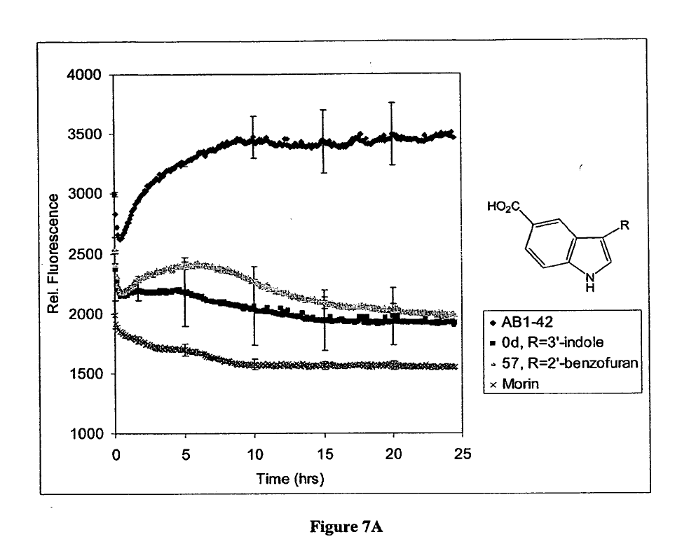

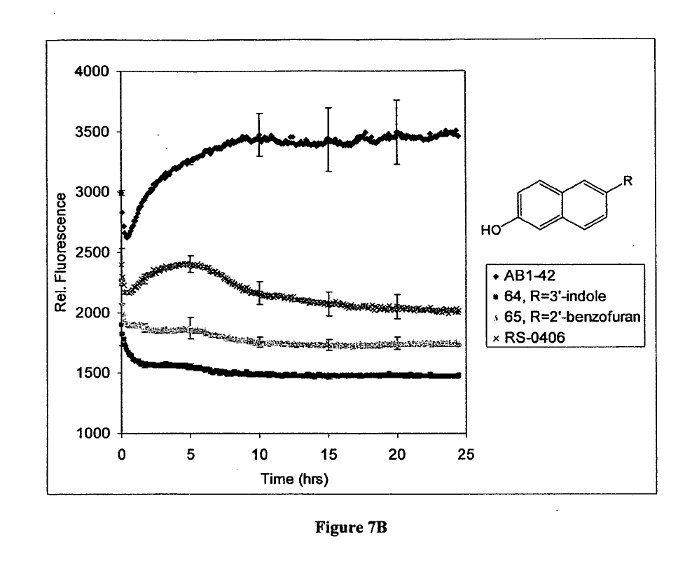

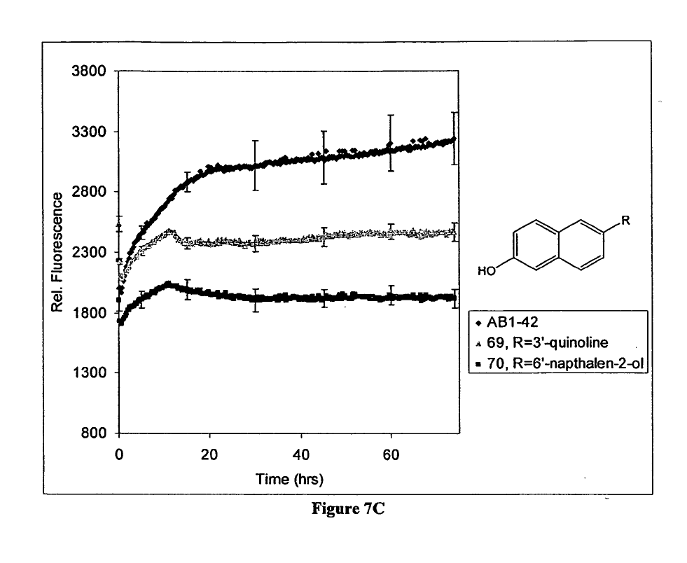

FIG. 7 depicts inhibition of Aβ¹⁻⁴² aggregation by various synthesized bi-aromatic compounds and positive controls morin (Ono K, Yoshiike Y, Takashima A, Hasegawa K, Naiki H, Yamada M. 2003. J Neurochem 87:172-81) and RS-0406 (Walsh D M, Townsend M, Podlisny M B, Shankar G M, Fadeeva J V, Agnaf O E, Hartley D M, Selkoe D J. 2005. J Neurosci 25:2455-62) in the kinetic ThT assay. Same conditions as for Aβ¹⁻⁴⁰, FIG. 1. Compound concentrations were 100 μM in (A), 20 μM in (B), except for RS-0406, which was tested at 100 μM, and 20 μM in (C).

FIG. 8 depicts dose-response curves for compounds 0j, 0k and 64 at inhibiting aggregation of Aβ¹⁻⁴² in the kinetic ThT assay.

### (ii) Circular Dichroism Studies

Aliquots (220 μL) of pretreated Aβ¹⁻⁴⁰ (40 μM in 20 mM Tris-HCl, pH 7.4) were added directly to 1 mm quartz circular dichroism (CD) cells, followed by 2.2 μL compound (of varying concentration) in methanol or methanol alone (controls). Solutions were incubated at 37° C. for up to 6 days. CD scans were performed on a J-810 spectropolarimeter (obtained from Jasco Corporation, Tokyo, Japan) between 190 and 250 nm, with a resolution of 0.1 nm and bandwidth of 1 nm. Ten scans were obtained for each reading. Spectra had background CD of the buffer subtracted and were left unsmoothed. Readings were performed at 37° C.

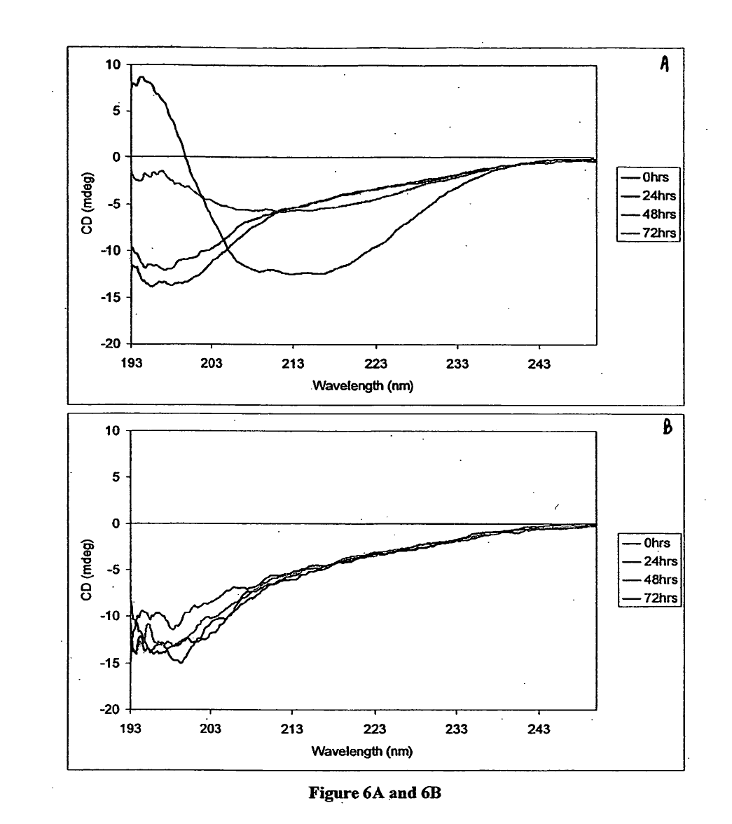

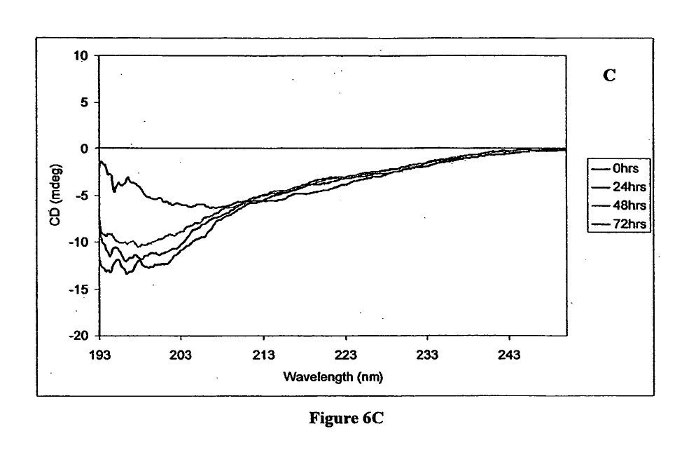

Aβ¹⁻⁴⁰, in the absence of any compound, shifted from primarily random coil (RC) at the beginning of an experiment to primarily β-sheet over the course of about 3 days (FIG. 6A). When a bis-indole compound was added (e.g. 0c, 0j), this transition was inhibited (FIG. 6B). In the case of 0j, the RC→β-sheet transition was inhibited to a greater extent than by the potent anti-amyloidogenic polyphenol morin (Ono K, Yoshiike Y, Takashima A, Hasegawa K, Naiki H, Yamada M. 2003. J Neurochem 87:172-81) (FIG. 6B, C).

FIG. 6 depicts circular dichroism (CD) of Aβ¹⁻⁴⁰ alone (A) and in the presence of 0j (B) and morin (C). Conditions: 40 μM Aβ¹⁻⁴⁰, 200 μM compound (B,C), pH 7.4, Tris-HCl (20 mM), 1% MeOH. Prior to experiment, Aβ¹⁻⁴⁰ pretreated with 1,1,1,3,3,3-hexafluoroisopropanol (HFIP).

### (iii) ¹H NMR Binding of Bi-Aromatics to Aβ¹⁻⁴⁰

Aβ¹⁻⁴⁰ was dissolved in HFIP (1 mL) and sonicated (20 min) to disassemble any pre-formed aggregates. The HFIP was removed with a stream of Ar(g) and the waxy residue dissolved in 2.2 mL buffer (Tris-d₁₁, 20 mM in D₂O). Incubations (0.500 mL) were made directly in thin-walled NMR tubes, to which were added compound (2.5 μL, 10 mM in DMSO-d₆) or vehicle. An identical incubation lacking Aβ¹⁻⁴⁰ was used as compound reference. Spectra were obtained for Aβ¹⁻⁴⁰ alone (100 μM), compound alone (50 μM) and for a mixture of both (100 μM Aβ¹⁻⁴⁰, 50 μM compound). Spectra were recorded at 500 MHz and 27° C. (300K), with 674 scans obtained for each.

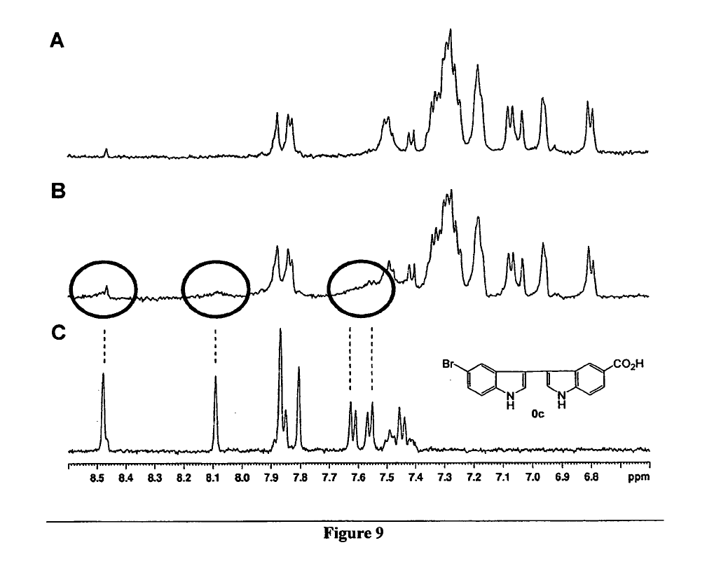

A subset of active compounds were evaluated in the ¹H NMR binding experiments (FIG. 9). The proton peaks of compounds experienced pronounced signal broadening in the presence of Aβ¹⁻⁴⁰ (2:1 peptide:compound), a phenomenon characteristic of ligand-protein binding (Zartler E R, Yan J, Mo H, Kline A D, Shapiro M J. 2003. Curr Topics Med Chem 3:25-37).

FIG. 9 depicts ¹H NMR spectra identifying binding of 5-bromo-5′-carboxy-3,3′-bis-indolyl (0c) to Aβ¹⁻⁴⁰. Spectra were obtained for Aβ¹⁻⁴⁰ alone (100 μM, A), compound alone (50 μM, C) and for a mixture of both (100 μM Aβ¹⁻⁴⁰, 50 μM compound, B). Spectra were recorded at 500 MHz and 27° C. (300K) in D₂O containing 1% DMSO-d6, and 674 scans were obtained for each.

### (iv) tau441 Aggregation

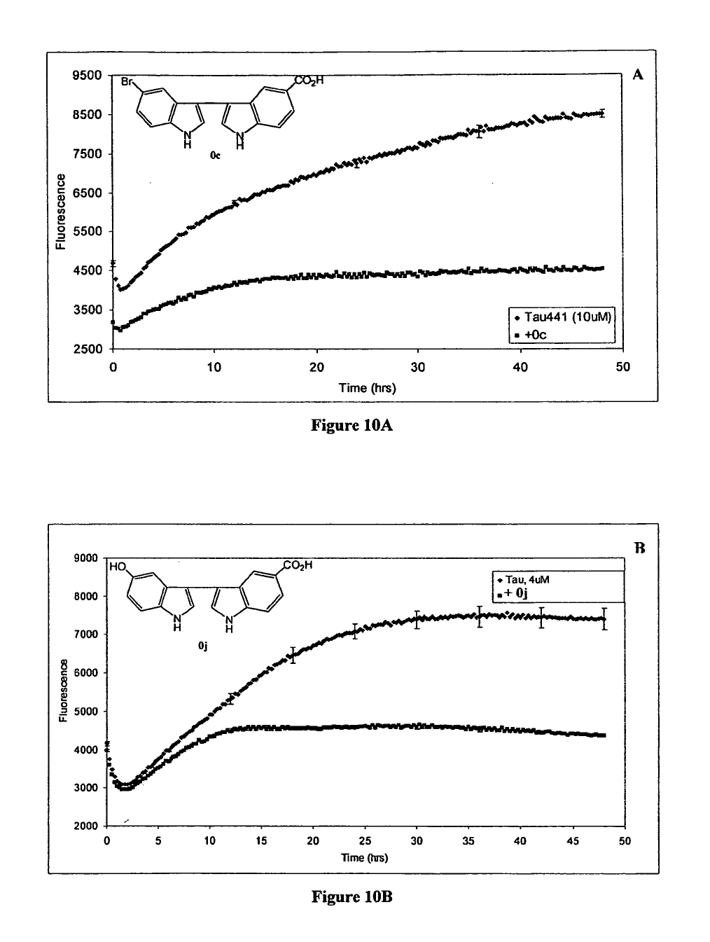

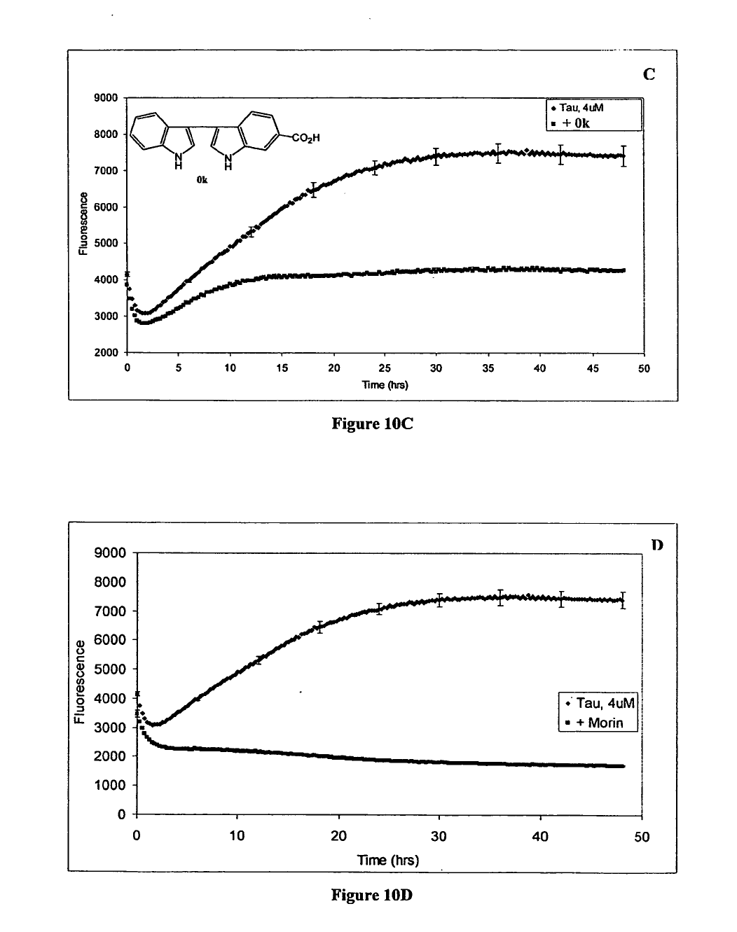

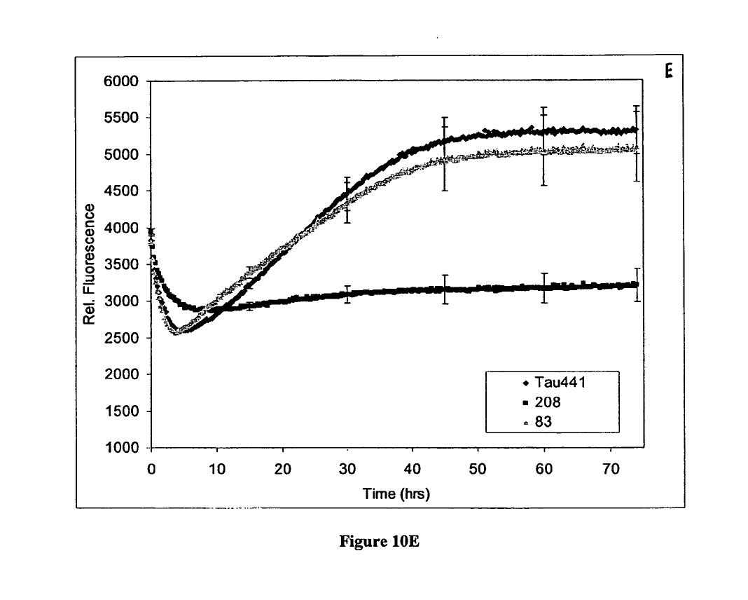

A kinetic ThS assay of tau441 aggregation was used to evaluate the bi-aromatic compounds, as detailed in Example 8, paragraph (i). Some synthesized compounds both reduced the rate of tau fibrillization and the equilibrium or plateau level of fibrillization (FIG. 10, A, B, C, E). This was also found to be the case for the positive control morin (FIG. 10D); polyphenols such as morin have been shown to inhibit tau fibrillization (Taniguchi S, Suzuki N, Masami M, Hisanga S, Iwatsubo T, Goedert M, Hasegawa M. 2005. J Biol Chem 280:7614-7623). All compounds in FIG. 10 inhibit Aβ aggregation in a Thioflavin T fluorescence assay and therefore have potential for a dual mechanism of action for ameliorating Alzheimer's disease.

FIG. 10 shows the inhibition of tau fibrillization by synthesized bi-aromatic compounds and morin. Aggregation conditions: tau441 at a concentration of either 10 μM (A) or 4 μM (B,C,D,E) was incubated in covered black 96-well polystyrene microplates alone or with 100 (A) or 50 μM compound (B,C,D,E), 5 μM ThS, pH 7.4, Tris-HCl (50 mM) containing NaN₃ (50 μM), and either 1% MeOH (A) or 1% DMSO (B,C,D,E). Plates were heated at 37° C. in a Tecan GENios microplate reader. All incubations performed in triplicate, except for compound 0c in (A), which had n=1. Fluorescence readings: fluorescence measured every 15 min. (λₑₓ=450 nm, λₑₘ=480 nm) after shaking for 15 sec. and pausing 10 sec. before taking measurement. Values are the mean of three replicates. Error bars, sometimes not visible due to small size, represent standard deviation of the mean.

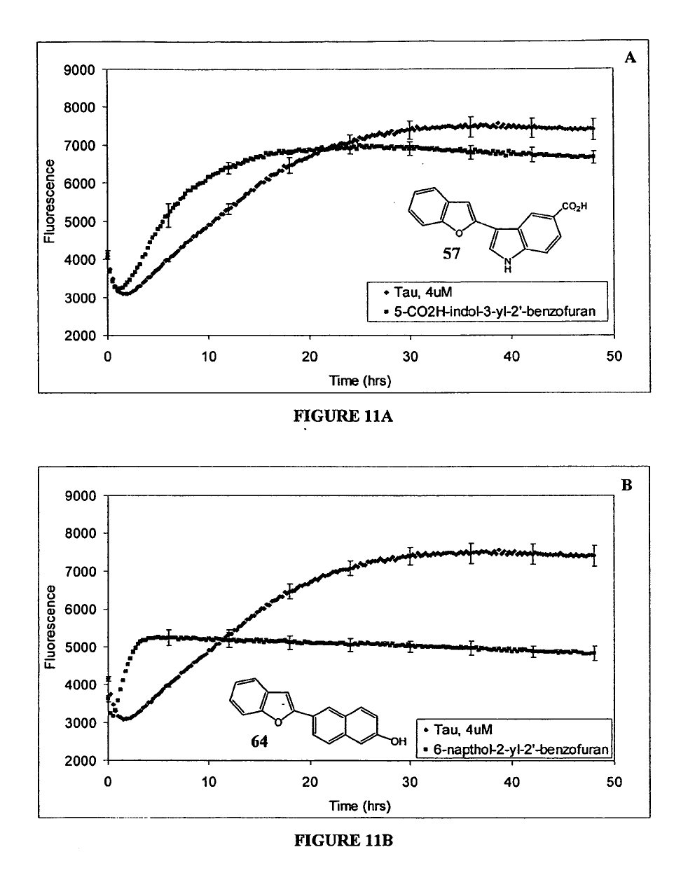

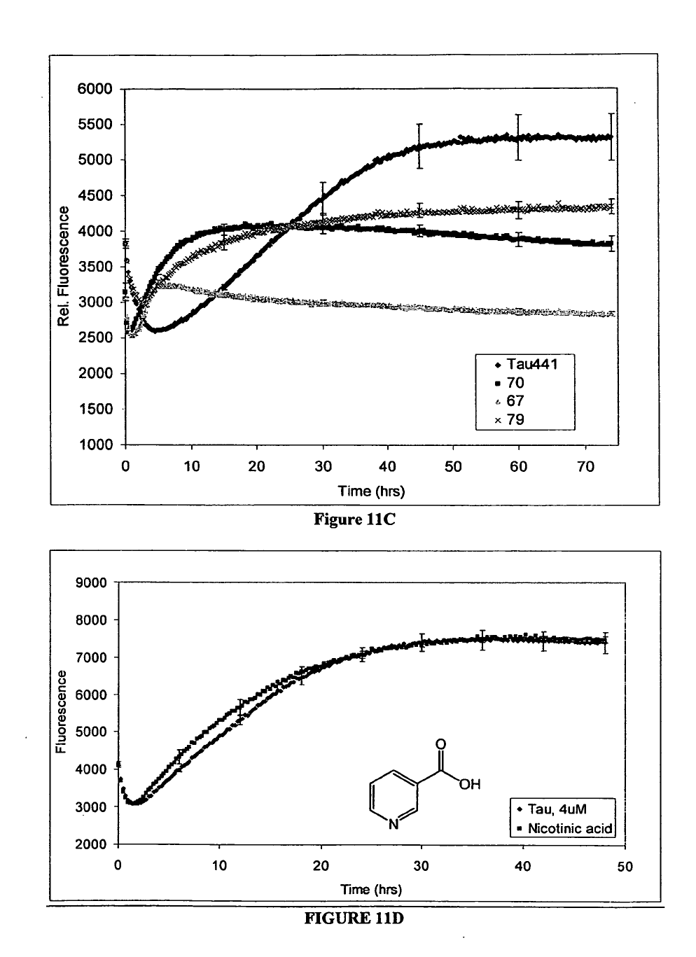

Unlike those compounds of FIG. 10, some compounds were found to increase the initial rate of tau aggregation, though had lower plateaus than the control (FIG. 11). The lower equilibrium level of aggregation may indicate a decreased amount of tau fibrillization and suggests these compounds may still have benefit in treating AD. As with FIG. 10, all compounds in FIG. 11 were found to inhibit Aβ aggregation. This demonstrates variable activity of the compounds against different misfolding proteins. In other words, a given bi-aromatic compound can inhibit two or more proteins/peptides from aggregating, or it may inhibit the aggregation of one while promoting that of another.

Other than 83 in FIG. 10E, all the bi-aromatics tested significantly modulated tau fibrillization at concentrations of 50 to 100 μM (FIGS. 10 and 11). Conversely, nicotinic acid, tested as a negative control, had no effect on tau fibrillization, even at a concentration of 1 mM (FIG. 11D). This demonstrates the specificity of binding of bi-aromatic compounds to tau.

FIG. 11 shows the effect on tau aggregation of synthesized bi-aromatic compounds and nicotinic acid. Aggregation conditions: tau441 at a concentration of 4 μM was incubated in covered black 96-well polystyrene microplates alone or with 50 μM (A,B,C) or 1 mM compound (D), 5 μM ThS, pH 7.4, Tris-HCl (50 mM) containing NaN₃ (50 μM), and 1% DMSO. Plates were heated at 37° C. in a Tecan GENios microplate reader. All incubations performed in triplicate. Fluorescence readings: fluorescence measured every 15 min. (λₑₓ=450 nm, λₑₘ=480 nm) after shaking for 15 sec. and pausing 10 sec. before taking measurement. Values are the mean of three replicates. Error bars, sometimes not visible due to small size, represent standard deviation of the mean.

Heparin is used as an inducer of aggregation in the ThS tau assay (FIGS. 10, 11). When it was omitted, tau aggregation was still found to occur, but at a much slower rate. Compound 0c and positive control morin both inhibited tau aggregation in the absence of heparin inducer.

### (v) α-Synuclein Aggregation

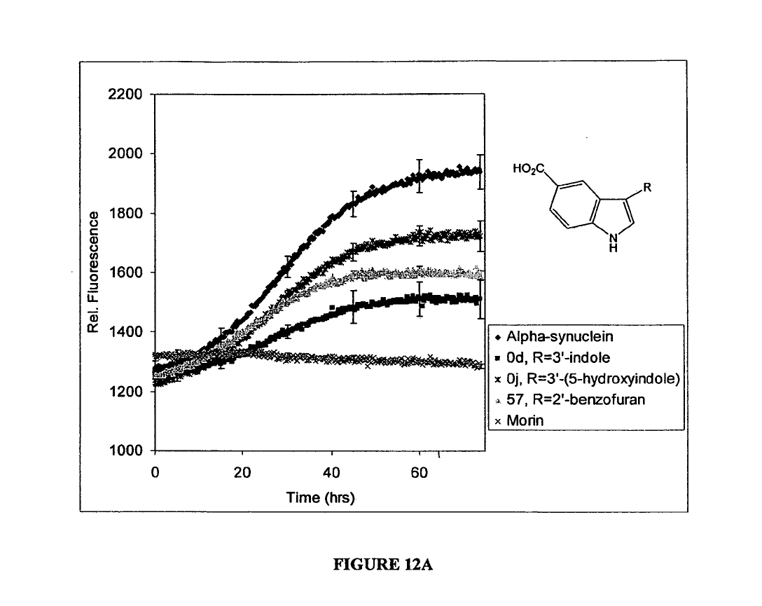

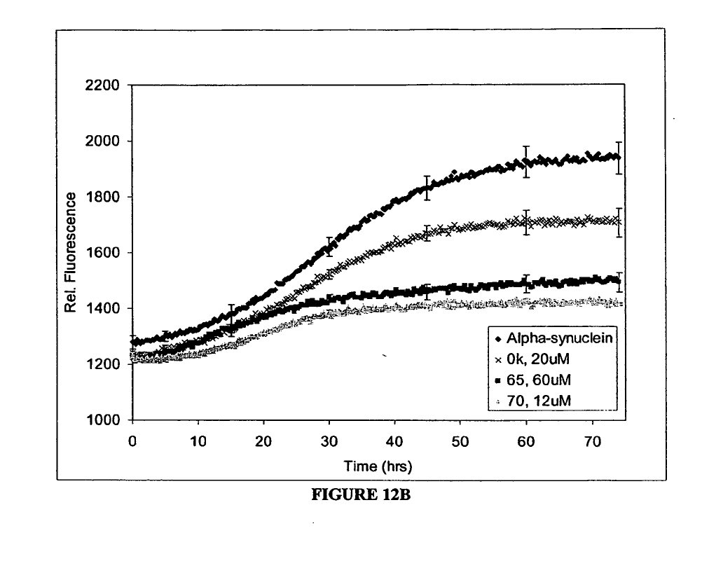

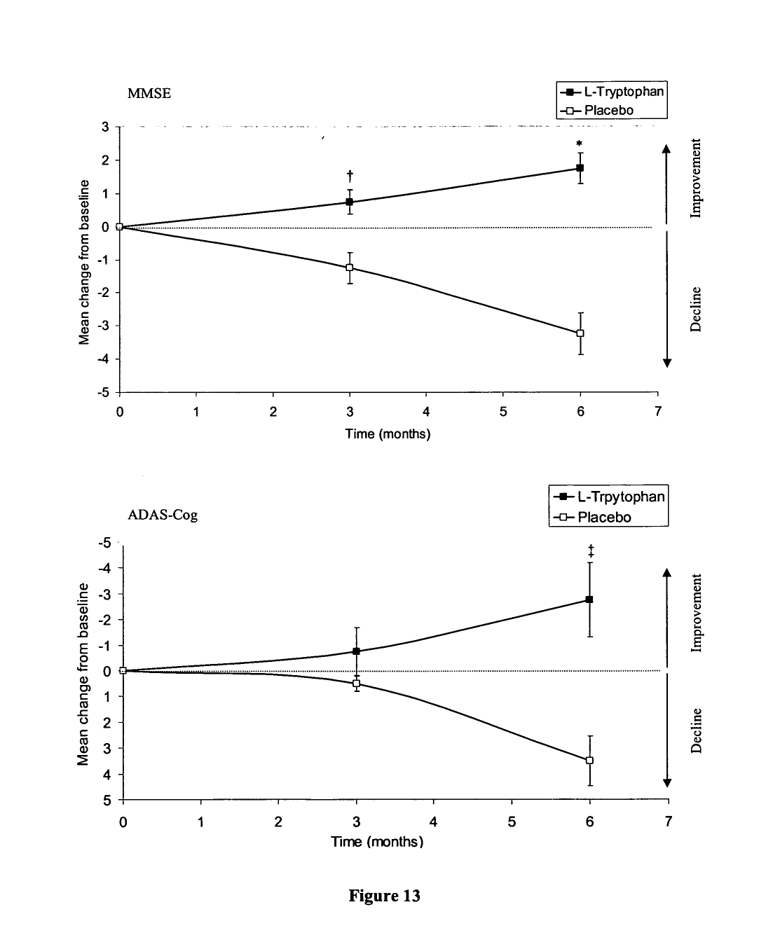

Aggregation of the α-synuclein protein has been implicated in the pathogenesis of a number of neurodegenerative conditions, including Alzheimer's disease, Lewy body diseases (e.g. Parkinson's disease) and multiple system atrophy (Ono K, Yamada M. 2006. J Neurochem 97:105-15). Inhibiting its aggregation is expected to be beneficial in treating these diseases. Compounds were evaluated for their ability to inhibit α-synuclein aggregation using a ThT fluorescence assay similar to that of Necula M, Chirita C N, Kuret J. 2003. J Biol Chem 21: 46674-80. Several of the synthesized bi-aromatic compounds, as well as positive controls, were found to be active in the assay (FIG. 12).

FIG. 12 depicts inhibition of α-synuclein aggregation by synthesized bi-aromatics and positive control morin (K, Yamada M. 2006. J Neurochem 97:105-15). Aggregation conditions: α-Synuclein (6 μM) was incubated in covered black 96-well polystyrene microplates in Tris-HCl (20 mM, pH 7.4), 10 μM ThT, 1% DMSO with compound concentrations of 100 μM (A) or as indicated (B). Plates were heated at 37° C. in a Tecan Genios microplate reader. All incubations were performed in triplicate. Fluorescence readings: fluorescence was measured every 15 min. (λₑₓ=450 nm, λₑₘ=480 nm) after shaking for 15 sec. and pausing 10 sec. before taking measurement. Values are the mean of three replicates. Error bars represent standard deviation of the mean.

### EXAMPLE 9

### Trp-Trp Dipeptides as Therapeutic Agents

The Trp-Trp dipeptides synthesized (Table 2) have two benefits as therapeutic agents: 1) They are unlikely to possess significant toxicity, given that they are composed of a naturally occurring amino acid and/or its enantiomer i.e. L- or D-tryptophan. 2) Due to their similarity to L-Trp they may be recognized by the large neutral amino acid transporter and thereby cross the blood-brain barrier. These two characteristics increase the likelihood of the dipeptides having favourable pharmacokinetics.

Certain Trp-Trp dipeptides in accordance with the present invention are listed in Table 2 below.

TABLE 2

| | |
|---|---|
| (a) | Linear Trp-Trp dipeptides |

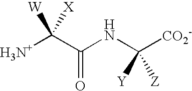

| | W | X | Y | Z |
|---|---|---|---|---|
| L-L | 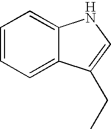 | H | H | 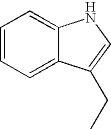 |
| L-D | 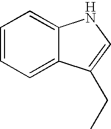 | H | 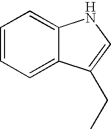 | H |
| D-L | H | 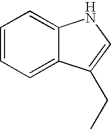 | H | 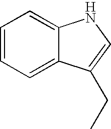 |
| D-D | H | 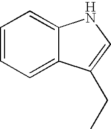 | 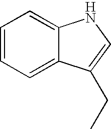 | H |

| | |
|---|---|
| (b) | Cyclic Trp-Trp dipeptides |

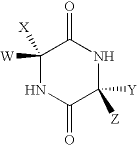

| | W | X | Y | Z |
|---|---|---|---|---|
| cyclo(L-L) | 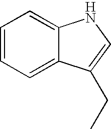 | H | H | 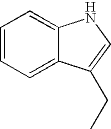 |
| cyclo(D-D) | H | 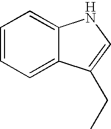 | 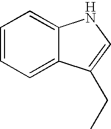 | H |
| meso-cyclo | 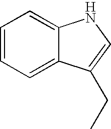 | H | 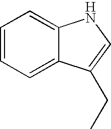 | H |

Since any anticipated drug-receptor binding is dependent on the three-dimensional structure of the drug's interacting functional groups, all combinations of L- and D-stereoisomers of Trp-Trp were synthesized (L-L, L-D, D-L, D-D). This served the dual purposes of increasing the likelihood of producing a successful interaction and providing possible clues as to the stereochemical requirements of binding. Incorporating the D-enantiomer of Trp into the molecule was also part of the overall peptidomimetic drug-design process. Since the D-enantiomer is non-naturally occurring, the chance of enzymatic amide bond cleavage of D-containing peptides is reduced, thereby resulting in improved pharmacokinetics over the L-L dipetide.

The synthesis of cyclic Trp-Trp dipeptides was also performed as a further step in the peptidomimetic process. Possessing neither an amino nor carboxyl terminus, these molecules have a reduced likelihood of being metabolized by endogenous proteases. Another attractive feature of cyclic dipeptides is their general lack of toxicity. Their cyclic structure limits conformational freedom and therefore reduces the number of non-target receptors with which they can interact. Finally, many cyclic peptides are capable of crossing the blood-brain barrier due to their lipophilicity and lack of zwitterionic termini, making them suitable for the treatment of neurological diseases.

### EXAMPLE 10

### Synthesis of Trp-Trp Dipeptides

The synthesis of the tryptophan dipeptides first involved coupling of Trp, protected at the N-terminal with the benzyloxycarbonyl (CBZ) group, to Trp protected at the C-terminal by a methyl ester. Coupling of one of the N-protected residues (either D or L) to one of the C-protected residues was carried out using the coupling reagent PyBOP and HOBt. (PyBOP=Benzotriazol-1-yl-oxytripyrrolidinophosphonium hexafluorophosphate; HOBt=N-Hydroxybenzotriazole). This step is shown in Scheme 20 of Example 10 for the formation of protected L-L. The protecting groups were then removed via the saponification of the methyl ester and catalytic hydrogenation of the CBZ group to give the linear dipeptides.

To produce cyclic dipeptides, catalytic hydrogenation was performed on diprotected Trp-Trp to remove the CBZ group, followed by a reflux in methanol to effect the intramolecular cyclization. The reflux was performed in the presence of the base 1,4-diazabicyclo[2.2.2]octane (DABCO) to enhance the nucleophilicity of the N-terminal nitrogen and thereby speed the reaction rate. Ammonia has been used in this capacity in earlier syntheses of cyclo(L-Trp-L-Trp). The saponification, hydrogenation, and cyclization reactions are presented in Scheme 21 of Example 10.

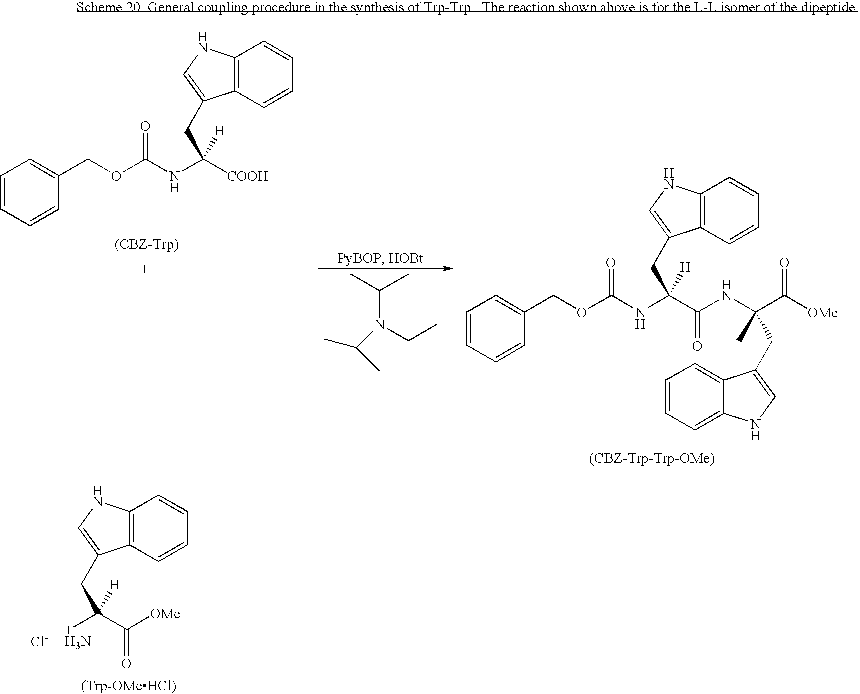

While a total of four stereoisomers exist for linear Trp-Trp (L-L, L-D, D-L and D-D), only three different configurations exist for cyclic Trp-Trp, namely cyclo(L-L), cyclo(D-D) and meso-cyclo(Trp-Trp); upon cyclization, the carboxyl and amino terminal residues become indistinguishable from one another and as a result, the L-D and D-L isomers are rendered identical. The three isomers of cyclic Trp-Trp are shown in Table 2 above and their general synthesis from the corresponding diprotected dipeptides is given in Scheme 21 of Example 10.

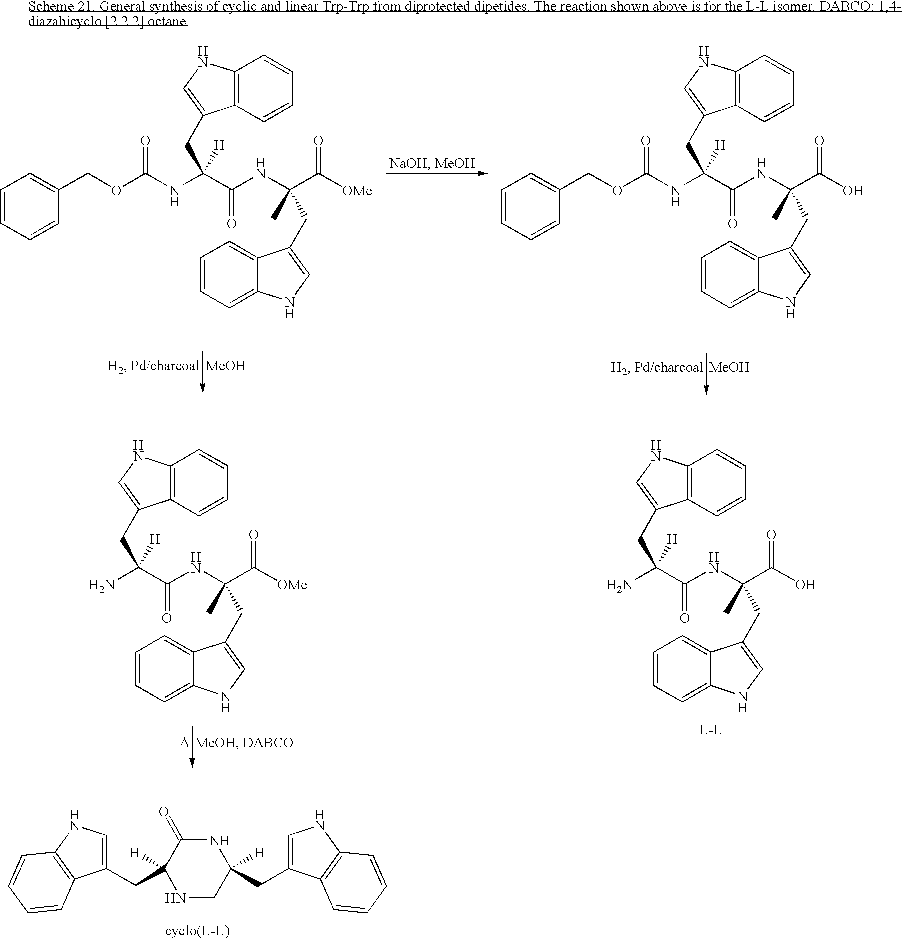

### EXAMPLE 11

### Experimental

Reagents and solvents were obtained from commercial sources (Aldrich, Bachem, and Fluka). Melting points (mp) were determined using a Mel-Temp II capillary apparatus and are uncorrected. Optical rotation was measured at the sodium D-line (589 nm), using an Autopol II automatic polarimeter with a path length of 1 decimeter; values are reported in units of degrees mL g⁻¹ dm⁻¹. Thin layer chromatography (TLC) was performed using coated Brinkmann silica gel 60 F₂₅₄ plates with aluminum backing. Solvent systems used for TLC are given in Table 3. Compounds were visualized using UV light. The presence of primary amine functional groups was determined by developing the plates with ninhydrin.

Infrared (IR) spectra were recorded on a Bomem 120 FT-IR spectrophotometer using KBr disks. ¹H and ¹³C NMR spectra were recorded with a Bruker Avance 400 MHz spectrometer in deuterated dimethylsulfoxide (DMSO-d₆). Chemical shifts (δ) are reported as parts per million downfield of tetramethylsilane (TMS) and are calibrated based on solvent peaks. Correlation spectroscopy techniques (COSY and HETCOR) were used to confirm structural connectivity.

Fast atom bombardment (FAB) mass spectrometry was performed on a VG Quattro mass spectrometer. Compounds were analyzed in 2% acetic acid in glycerol. High performance liquid chromatography (HPLC) was performed on a System Gold apparatus from Beckman fitted with a C₁₈ reverse phase column. Methanol and 0.2% trifuoroacetic acid (TFA), both HPLC grade, were used for the solvent system and compound detection was achieved by monitoring absorbance at 220 nm. All compounds synthesized were greater than 95% pure using the HPLC method outlined above.

TABLE 3
Solvent systems used for TLC.

| Solvent system | Solvents | Ratio |
|---|---|---|
| A | Acetonitrile/water/acetic acid | 20:1:1 |
| B | Ethanol/acetic acid | 50:1 |
| C | Acetonitrile/water/acetic acid | 4:1:1 |
| D | Ethyl acetate/methanol | 5:1 |

### EXAMPLE 12

### General Procedure for Coupling of CBZ-Trp and Trp-OMe

The N-protected Trp (0.679 g, 2.01 mmol, 1.0 eq.) was dissolved in THF (30 mL). PyBOP (1.0 eq.) was added followed by HOBt (1.0 eq.) and the carboxylic acid protected amino acid salt (0.563 g, 2.20 mmol, 1.1 eq.). Diisopropylethylamine (2.1 eq.) was added and the mixture stirred for 24 hrs (TLC completion). The solution was concentrated under reduced pressure and the resulting oil was dissolved in ethyl acetate (EtOAc, 25 mL). The organic phase was washed with brine (3×20 mL), 1 M HCl (2×20 mL), saturated NaHCO₃ (3×20 mL) and finally with brine again (3×20 mL). The organic phase was dried over MgSO₄, filtered and concentrated. The resulting yellow oil was recrystallized from EtOAc/hexanes and dried in vacuo, yielding a white solid. The yields, melting points and optical rotations for the compounds are shown in Table 4, along with literature values (all four diasteromers have been previously synthesized and characterized in Guarnaccia, et al., Biopolymers 1975, 14, 2329-2346; Chieu, et al., Journal of the American Chemical Society 1982, 104, 3002-3007). Other physical properties of the diprotected dipeptides (e.g. spectra from ¹H and ¹³C NMR, IR and mass spectrometry) were as expected and conformed to literature findings.

TABLE 4
Physical properties of CBZ-Trp-Trp-OMe isomers.

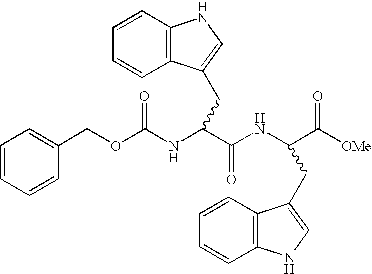

CBZ-Trp-Trp-OMe

| | |
|---|---|
| Melting | |
| point (° C.) | [α]_{D}²⁵ |

| Compound | Yield | Found | Lit. | Found | Lit. |
|---|---|---|---|---|---|
| CBZ-L-Trp-L-Trp-OMe | 86% | 192-194 | 194-197 | +23.5 | +27.1 |
| CBZ-L-Trp-D-Trp-OMe | 87% | 197-201 | 199-201 | −13.9 | — |
| CBZ-D-Trp-L-Trp-OMe | 92% | 211-214 | 205-206 | +14.2 | — |
| CBZ-D-Trp-D-Trp-OMe | 89% | 192-194 | 196-198 | −21.2 | — |

All values of [α]_{D}²⁵ measured in glacial acetic acid (AcOH), at a concentration of 0.8 g/dl
(See Chieu DT et al. 1982. J Am Chem Soc 104: 3002-7)
(See also Guaraccia R et al. 1975. Biopolymers 14: 2329-46)

### EXAMPLE 13

### General Procedure for Removal of Methyl Ester from the C-Terminal of Trp-Trp

The saponification method of McDermott et al., Journal of the American Chemical Society 1982, 104, 3002-3007 was used. The diprotected dipeptide (0.425 g, 0.79 mmol) was dissolved in MeOH (20 mL) and 1 N NaOH (5 mL) was added to the solution. On occasion, the reaction was performed on double or triple this scale without difficulty. The reaction was stirred and monitored by TLC. After 2 hrs., water (20 mL) was added and unreacted compound was removed by washing with ether (2×20 mL). The aqueous layer was then acidified to pH 2 with conc. HCl and EtOAc was quickly added (20 mL). The EtOAc layer was separated and the aqueous layer extracted with two more portions of EtOAc (20 mL). The extracts were dried over MgSO₄, concentrated and the resulting oil was recrystallized from EtOAc/hexanes and then from MeOH/H₂O. The white solid obtained was dried in vacuo.

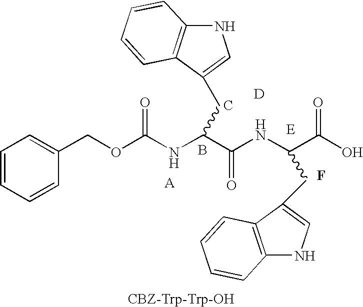

The four diastereomers of the N-protected dipeptides (CBZ-Trp-Trp-OH) in accordance with the present invention were as follows:

CBZ-L-Trp-L-Trp-OH

White solid (0.257 g, 87.3%); mp 207-210° C. (lit. 209-210° C.); [α]_{D}²⁵ +19.2, c 0.8, AcOH (lit. +23.7, c 0.8, AcOH); TLC (R_{f}) A: 0.74, B: 0.69; IR (vₘₐₓ): 3412 (NH), 3372 (NH), 3334 (NH), 3054 (CH, aromatic), 2921 (CH), 1729 (COOH), 1695 (C═O), 1659 (C═O), 1515 (C═C, aromatic) cm⁻¹; ¹H NMR: δ: 2.90 (dd, 1H, H_{C}, J=11.7 Hz, J=12.1 Hz), 3.21 (m, 3H, 2H_{F} and H_{C′}), 4.35 (m, 1H, H_{B}), 4.54 (m, 1H, H_{E}), 4.94 (s, 2H, Z-CH₂), 7.2 (m, 14H, 13Ar—H and H_{A}), 7.56 (d, 1H, Ar—H, J=7.4 Hz), 7.65 (d, 1H, Ar—H, J=7.5 Hz), 8.29 (d, 1H, H_{D}, J=7.1 Hz), 10.82 (s, 1H, indole N—H), 10.90 (s, 1H, indole N—H), 12.64 (bs, 1H, CO₂—H); ¹³C NMR: 28.02, 28.69, 53.82, 56.19, 66.09, 110.49, 111.05, 112.16 (2C), 116.70 (2C), 119.04, 119.25, 119.42 (2C), 121.68, 121.78, 124.69 (2C), 128.12, 128.31, 128.51, 129.15, 136.91, 137.81, 153.21 (2C), 156.64, 172.81, 174.13; FAB (m/z): 525.2 [M+H]⁺.

CBZ-L-Trp-D-Trp-OH

White solid (0.799 g, 81.6%); mp 184-186° C.; [α]_{D}²⁵-11.7, c 0.8, AcOH; TLC (R_{f}) A: 0.74, B: 0.69; IR (vₘₐₓ): 3409 (NH), 3334 (NH), 3058 (CH, aromatic), 2934 (CH), 1715 (COOH), 1667 (C═O), 1515 (C═C, aromatic) cm⁻¹: ¹H NMR δ: 2.72 (dd, 1H, H_{C}, J=14.4 Hz, J=10.3 Hz), 2.90 (dd, 1H, H_{C′}, J=10.6 Hz, J=3.1 Hz), 3.03 (dd, 1H, H_{F}, J=8.5 Hz, J=14.5 Hz), 3.17 (dd, 1H, H_{F′}, J=4.9 Hz, J=14.4 Hz), 4.33 (m, 1H, H_{B}), 4.50 (m, 1H, H_{E}), 4.92 (s, 2H, Z-CH₂), 7.2 (m, 14H, 13Ar—H and H_{A}), 7.55 (d, 1H, Ar—H, J=7.8 Hz), 7.60 (d, 1H, Ar—H, J=7.9 Hz), 8.32 (d, 1H, H_{D}, J=7.9 Hz), 10.74 (s, 1H, indole N—H), 10.85 (s, 1H, indole N—H), 12.71 (bs, 1H, CO₂—H); ¹³C NMR: 28.02, 28.69, 53.69, 56.08, 65.97, 110.49, 110.95, 111.97, 112.10, 118.92, 118.99, 119.16, 119.44, 121.54, 121.68, 124.50, 124.61, 127.95, 128.03, 128.19, 128.40, 129.05 (2C), 136.79, 136.85, 137.75 (2C), 156.51, 172.54, 174.07; FAB (m/z): 525.2 [M+H]⁺.

CBZ-D-Trp-L-Trp-OH

White solid (0.829 g, 83.5%); mp 189-191° C.; [α]_{D}²⁵+12.4, c 0.8, AcOH; TLC (R_{f}) A: 0.74, B: 0.69; IR (vₘₐₓ): 3407 (NH), 3334 (NH), 3057 (CH, aromatic), 2926 (CH), 1714 (COOH), 1666 (C═O), 1517 (C═C, aromatic) cm⁻¹: ¹H NMR δ: 2.72 (dd, 1H, H_{C}, J=14.4 Hz, J=10.4 Hz), 2.90 (dd, 1H, H_{C′}, J=3.7 Hz, J=14.4 Hz), 3.04 (dd, 1H, J=8.5 Hz, J=14.5 Hz), 3.17 (dd, 1H, J=4.9 Hz, J=14.4 Hz), 4.33 (m, 1H, H_{B}), 4.51 (m, 1H, H_{E}), 4.92 (s, 2H, Z-CH₂), 7.2 (m, 14H, 13Ar—H and H_{A}), 7.55 (d, 1H, Ar—H, J=7.8 Hz), 7.60 (d, 1H, Ar—H, J=7.9 Hz), 8.32 (d, 1H, H_{D}, J=7.9 Hz), 10.74 (s, 1H, indole N—H), 10.85 (s, 1H, indole N—H), 12.71 (bs, 1H, CO₂—H); ¹³C NMR: 28.03, 28.70, 53.69, 56.09, 65.97, 110.49, 110.95, 111.97, 112.11, 118.92, 118.99, 119.16, 119.44, 121.54, 121.68, 124.50, 124.61, 127.95, 128.03, 128.19, 128.40, 128.58, 129.05, 136.79, 136.86, 137.75 (2C), 156.51, 172.55, 174.08; FAB (m/z): 525.2 [M+H]⁺.

CBZ-D-Trp-D-Trp-OH

White solid (0.838 g, 86.2%); mp 206-207° C.; [α]_{D}²⁵ −17.5, c 0.8, AcOH; TLC (R_{f}) A: 0.74, B: 0.69; IR (vₘₐₓ): 3409 (NH), 3334 (NH), 3057 (CH, aromatic), 2934 (CH), 1716 (COOH), 1663 (C═O), 1519 (C═C, aromatic) cm⁻¹; ¹H NMR: δ: 2.90 (dd, 1H, H_{C}, J=4.0 Hz, J=14.6 Hz), 3.21 (m, 3H, 2H_{F} and H_{C′}), 4.35 (m, 1H, H_{B}), 4.54 (m, 1H, H_{E}), 4.94 (s, 2H, Z-CH₂), 7.2 (m, 14H, 13Ar—H and H_{A}), 7.56 (d, 1H, Ar—H, J=7.4 Hz), 7.65 (d, 1H, Ar—H, J=7.8 Hz), 8.29 (d, 1H, H_{D}, J=7.4 Hz), 10.82 (s, 1H, indole N—H), 10.90 (s, 1H, indole N—H), 12.73 (bs, 1H, CO₂—H); ¹³C NMR: 27.87, 28.63, 53.82, 56.21, 66.09, 110.49, 111.04, 112.21 (2C), 116.67 (2C), 119.04, 119.25, 119.43 (2C), 121.69, 121.78, 124.52, 124.70, 128.12, 128.31, 128.51, 129.16, 136.91, 137.81, 152.69, 155.01, 156.64, 172.82, 174.14; FAB (m/z): 525.2 [M+H]⁺.

### EXAMPLE 14

### General Procedure for Removal of CBZ Group from the N-Terminal of Trp-Trp

The CBZ group was removed using catalytic hydrogenation. The N-protected dipeptide (0.470 g, 0.89 mmol) was dissolved in MeOH (20 mL) and the system flushed with N₂. Next, 10% palladium on charcoal (0.470 g) was added and the system was flushed with N₂ followed by H₂. The mixture was then stirred vigorously under H₂ pressure from a balloon until TLC indicated completion (approximately 1 hour). The solution was filtered and concentrated. The resulting clear oil was recrystallized from MeOH/Et₂O/hexanes, and dried in vacuo, giving a white or light pink powder.

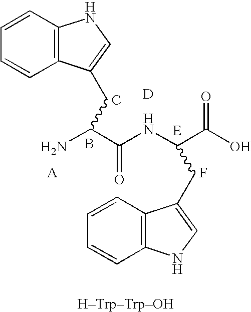

The four diastereomers of H-Trp-Trp-OH in accordance with the present invention were as follows:

H-L-Trp-L-Trp-OH

White solid (0.285 g, 81.1%); mp 174-176° C. (lit. 186° C.); [α]_{D}²⁵ −9.0, c 0.8, EtOH (lit.-11.0, c 0.426, EtOH); TLC (R_{f}) B: 0.30, C: 0.71; IR (vₘₐₓ): 3405 (NH), 3059 (CH, aromatic), 2927 (CH), 1668 (C═O), 1600 (C═O), 1522 (C═C, aromatic) cm⁻¹: ¹H NMR δ: 2.84 (dd, 1H, H_{C}, J=8.5 Hz, J=14.9 Hz), 3.12 (m, 3H, 2H_{F} and H_{C′}), 3.62 (dd, 1H, H_{B}, J=4.4 Hz, J=8.2 Hz), 4.51 (m, 1H, H_{E}), 5.9 (bs, 2H, H_{A}), 7.2 (m, 10H, Ar—H), 8.35 (d, 1H, H_{D}, J=7.4 Hz), 10.84 (s, 1H, indole N—H), 10.97 (s, 1H, indole N—H); ¹³C NMR: 28.25, 30.37, 54.15, 55.12, 110.24, 110.74, 111.98, 112.16, 118.92, 119.06, 119.27 (2C), 121.49, 121.71, 124.37, 125.01, 128.19, 128.38, 136.77, 137.09, 173.02, 174.16; FAB (m/z): 391.3 [M+H]⁺.

H-L-Trp-D-Trp-OH

Light pink solid (0.261 g, 75.0%); mp 173-176° C.; [α]_{D}²⁵ +32.0, c 0.8, EtOH; TLC (R_{f}) B: 0.17, C: 0.62; IR (vₘₐₓ): 3407 (NH), 3059 (CH, aromatic), 2927 (CH), 2855 (CH), 1668 (C═O), 1600 (C═O), 1526 (C═C, aromatic) cm⁻¹; ¹H NMR δ: 2.69 (dd, 1H, H_{C}, J=9.1 Hz, J=14.4 Hz), 3.04 (m, 2H, H_{C′} and H_{F}), 3.19 (dd, 1H, H_{F′}, J=5.5 Hz, J=15.1 Hz), 3.73 (m, 1H, H_{B}), 4.45 (m, 1H, H_{E}), 6.11 (bs, 2H, H_{A}), 7.3 (m, 10H, Ar—H), 8.37 (s, 1H, H_{D}), 10.82 (s, 1H, indole N—H), 10.92 (s, 1H, indole N—H); ¹³C NMR: 28.47, 29.98, 54.74 (2C), 109.90, 111.21, 111.98, 112.11, 118.89, 119.01, 119.23, 119.29, 121.44, 121.67, 124.35, 125.07, 127.99, 128.41, 136.80, 137.04, 171.98, 174.60; FAB (m/z): 391.3 [M+H]⁺.

H-D-Trp-L-Trp-OH

Light pink solid (0.310 g, 85.0%): mp 179-182° C.: [α]_{D}²⁵ −40.3, c 0.8, EtOH; TLC (R_{f}) B: 0.17, C: 0.62; IR (vₘₐₓ): 3407 (NH), 3262 (NH), 3059 (CH, aromatic), 2921 (CH), 1671 (C═O), 1600 (C═O), 1526 (C═C, aromatic) cm⁻¹: ¹H NMR δ: 2.68 (dd, 1H, H_{C}, J=9.0 Hz, J=14.2 Hz), 3.02 (m, 2H, H_{C′} and H_{F}), 3.18 (dd, 1H, H_{F′}, J=4.4 Hz, J=14.1 Hz), 3.71 (m, 1H, H_{B}), 4.44 (m, 1H, H_{E}), 5.50 (bs, 2H, H_{A}), 7.3 (m, 10H, Ar—H), 8.37 (s, 1H, H_{D}), 10.81 (s, 1H, indole N—H), 10.90 (s, 1H, indole N—H); ¹³C NMR: 28.41, 30.09, 54.64, 54.76, 109.94, 111.10, 111.99, 112.10, 118.91, 119.01, 119.20, 119.28, 121.46, 121.67, 124.36, 125.04, 127.99, 128.36, 136.80, 137.03, 172.14, 174.47; FAB (m/z): 391.3 [M+H]⁺.

H-D-Trp-D-Trp-OH

White solid (0.351 g, 73.1%); mp 172-175° C.: [α]_{D}²⁵ +11.0, c 0.8, EtOH; TLC (R_{f}) B: 0.30, C: 0.71; IR (vₘₐₓ: 3406 (NH), 3059 (CH, aromatic), 2927 (CH), 2868 (CH), 1668 (C═O), 1600 (C═O), 1523 (C═C, aromatic) cm⁻¹: ¹H NMR δ: 2.82 (dd, 1H, H_{C}, J=8.5 Hz, J=14.2 Hz), 3.13 (m, 3H, 2H_{F} and H_{C′}), 3.62 (m, 1H, H_{B}), 4.52 (m, 1H, H_{E}), 4.70 (bs, 2H, H_{A}), 7.2 (m, 10H, Ar—H), 8.39 (d, 1H, H_{D}, J=6.8 Hz), 10.83 (s, 1H, indole N—H), 10.94 (s, 1H, indole N—H); ¹³C NMR: 28.27, 30.38, 54.15, 55.12, 110.26, 110.75, 111.98, 112.16, 118.93, 119.08, 119.27 (2), 121.50, 121.71, 124.37, 125.04, 128.20, 128.38, 136.78, 137.09, 173.03, 174.16; FAB (m/z): 391.3 [M+H]⁺.

### EXAMPLE 15

### General Procedure for Dipeptide Cyclization

Diprotected dipeptides (0.545 g, 1.00 mmol) were first catalytically hydrogenated in order to remove the CBZ protecting group from the N-terminal. This was performed according to the preceding procedure, with the exception that the final recrystallization was not performed. Instead, the oil was dissolved in MeOH (200 mL) and refluxed with DABCO (0.380 g, 3 eq.) until TLC indicated completion (24 to 48 hrs). The solution was concentrated, leaving a clear oil. Water (50 mL) was added followed by dropwise addition of 1N HCl until the pH reached 2. The resulting white or light yellow precipitate was filtered, washed with water (50 mL×3), washed with ether (20 mL×3) and was dried in vacuo.

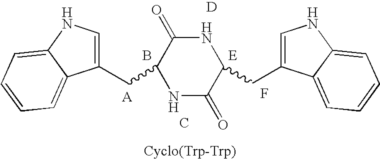

The three diastereomers of cyclo(Trp-Trp) of Example 15 in accordance with the present invention were as follows:

Cyclo(L-Trp-L-Trp)

White solid (0.225 g, 59.5%); mp 271-276° C. (lit. 265-268° C.); [α]_{D}²⁵ −141, c 0.8, MeOH (lit.-115, c 0.32, MeOH); TLC (R_{f}) A: 0.66, D: 0.47; IR (vₘₐₓ): 3413 (NH), 3328 (NH), 3056 (CH, aromatic), 2927 (CH), 1737 (N—C=Q), 1671 (N—C═O), 1516 (C═C, aromatic) cm⁻¹; ¹H NMR δ: 2.18 (m, 2H, H_{A} and H_{F}), 2.70 (m, 2H, H_{A′} and H_{F′}), 3.88 (m, 2H, H_{B} and H_{E}), 6.60 (s, 2H, Ar—H), 7.00 (m, 4H, Ar—H), 7.31 (m, 4H, Ar—H), 7.72 (s, 2H, H_{C} and H_{D}), 10.85 (s, 2H, indole N—H); ¹³C NMR: 30.87 (2C), 56.06, 56.14, 109.61 (2C), 112.12 (2C), 119.21 (2C), 119.40 (2C), 121.66 (2C), 125.26 (2C), 128.21 (2), 136.84, 136.90, 167.59 (2C); FAB (m/z): 373.3 [M+H]⁺.

Meso-cyclo(Trp-Trp)

Light yellow solid (0.268 g, 71.8%); mp 237-239; [α]_{D}²⁵ +1.3, c 0.8, MeOH; TLC (R_{f}): A: 0.64, D: 0.47; IR (vₘₐₓ): 3413 (NH), 3347 (NH), 3056 (CH, aromatic), 2927 (CH), 1735 (N—C═O), 1672 (N—C═O), 1516 (C═C, aromatic) cm⁻¹; ¹H NMR δ: 2.85 (dd, 2H, H_{A} and H_{F}, J=14.4 Hz, J=4.2 Hz), 3.10 (dd, 2H, H_{A′} and H_{F′}, J=14.4 Hz, J=3.7 Hz), 3.37 (m, 2H, H_{B} and H_{E}), 7.0 (m, 6H, Ar—H), 7.30 (d, 2H, Ar—H, J=8.0 Hz), 7.51 (d, 2H, Ar—H, J=7.8 Hz), 7.85 (s, 2H, H_{C} and H_{D}), 10.86 (s, 2H, indole N—H); ¹³C NMR: 29.04 (2C), 55.40 (2C), 109.14 (2C), 111.85 (2C), 119.06 (2C), 119.64 (2C), 121.53 (2C), 125.21 (2C), 128.39 (2C), 136.58 (2C), 168.37 (2C); FAB (m/z): 373.2 [M+H]⁺.

Cyclo(D-Trp-D-Trp)

White solid (0.241 g, 62.0%); mp 276-278° C.; [α]_{D}²⁵ +1200, c 0.8, EtOH; TLC (R_{f}) A: 0.69, D: 0.49; IR (vₘₐₓ): 3419 (NH), 3328 (NH), 3059 (CH, aromatic), 2927 (CH), 1735 (N—C═O), 1671 (N—C═O), 1516 (C═C, aromatic) cm⁻¹: ¹H NMR δ: 2.17 (m, 2H, H_{A} and H_{F}), 2.70 (m, 2H, H_{A′} and H_{F′}), 3.88 (m, 2H, H_{B} and H_{E}), 6.60 (s, 2H, Ar—H), 7.02 (m, 4H, Ar—H), 7.31 (m, 4H, Ar—H), 7.71 (s, 2H, H_{C} and H_{D}), 10.84 (s, 2H, indole N—H); ¹³C NMR: 30.85 (2C), 56.13 (2C), 109.61 (2C), 112.11, 112.37, 119.21 (2), 119.40 (2), 121.66 (2C), 125.26 (2C), 128.20 (2C), 136.90 (2C), 167.59 (2C).

### EXAMPLE 16

A phase IV clinical trial of L-tryptophan (Trp) was carried out to evaluate the amino acid's effect on cognitive abilities in patients with mild to moderate Alzheimer's disease (AD). An earlier trial has been performed which studied Trp administration in patients suffering from AD [Bentham, P W. International Clinical Psychopharmacology, 1990, 5: 261-72]. The present trial expects the possible binding of Trp to the HHQK region of Aβ [D Giulian et al. (1998). Journal of Biological Chemistry 273:29719-26] and subsequent disruption of Aβ plaque formation, whereas the earlier trial was based on neurotransmitter replacement therapy. By administering Trp, levels of its metabolite 5-hydroxytryptophan, better known as serotonin, were expected to increase, thereby countering deficits of the neurotransmitter experienced by those suffering from AD [Siegel, G J; Agranoff, B W; Albers, R W; Molinoff, P B, Basic Neurochemistry. Fifth ed. 1994, New York: Raven Press, 1054 pp]. Also providing motivation for the trial may have been earlier accounts that Trp was seen to benefit elderly patients with mental disorders [Shaw, D M; Tidmarsh, S F; Karajgi, B M; Sweeney, E A; Williams, S; Elameer, M; Twining, C. British Journal of Psychiatry, 1981, 139: 580-2, Lehmann, J; Persson, S; Walinder, J; Wallin, L. Acta Psychiatrica Scandinavica, 1981, 64: 123-31].

The earlier placebo-controlled Trp trial [Bentham, P W. International Clinical Psychopharmacology, 1990, 5: 261-72] suffered from several factors that hindered the generation and interpretation of data. These included a small sample size of only ten patients, two of which dropped out during the trial; problems with the sensitivity of their cognitive and behavioural tests; a relatively short observation period of three months; and the finding that their placebo group was substantially more demented at baseline. Despite these confounding features, however, the trial yielded a statistically significant correlation between the increase in Trp plasma levels and the increase in cognitive test scores. This finding, that cognition improved as a function of circulating Trp levels, was interpreted positively by the trial's lead investigator and author. In his concluding remarks, however, the author speculated that further examination of Trp's benefits in AD were unlikely to be pursued on a larger scale due to the then-recent implication of herbal supplements of Trp in the potentially fatal eosinophilia myalgia syndrome (EMS). As discussed in Section 2.3 of the protocol of Appendix A, the cases of EMS were eventually linked to a single company that produced Trp, along with an inadvertent toxic by-product, using genetically modified bacteria.

The prediction that studies using Trp would be hampered by concerns over EMS proved true, and no further clinical trials of Trp supplementation in AD have been published in the 15 plus years since its potentially beneficial effects were identified. Recent trials, however, have shown that acute Trp depletion in patients with AD caused impairment in cognitive function [Porter, R J; Lunn, B S; Walker, L L; Gray, J M; Ballard, C G; O Brien, J T. American Journal of Psychiatry, 2000, 157: 638-40, Porter, R J; Lunn, B S; O'Brien, J T. Psychol Med, 2003, 33: 41-9]. This finding, coupled with the discovery that AD patients have significantly reduced plasma levels of Trp [Fekkes, D; van der Cammen, T J; van Loon, C P; Verschoor, C; van Harskamp, F; de Koning, I; Schudel, W J; Pepplinkhuizen, L. J Neural Transm, 1998, 105: 287-94, Widner, B; Leblhuber, F; Walli, J; Tilz, G P; Demel, U; Fuchs, D. Adv Exp Med Biol, 1999, 467: 133-8, Widner, B; Leblhuber, F; Walli, J; Tilz, G P; Demel, U; Fuchs, D. J Neural Transm, 2000, 107: 343-53] support a protective role for the amino acid in the aetiology of AD. The authors of the studies often suggest that a depletion of serotonin levels, resulting from reduced levels of circulating Trp, may contribute significantly to the cognitive impairment of AD [Bentham, P W. International Clinical Psychopharmacology, 1990, 5: 261-72., Porter, R J; Lunn, B S; Walker, L L; Gray, J M; Ballard, C G; O Brien, J T. American Journal of Psychiatry, 2000, 157: 638-40, Porter, R J; Lunn, B S; O'Brien, J T. Psychol Med, 2003, 33: 41-9, Fekkes, D; van der Cammen, T J; van Loon, C P; Verschoor, C; van Harskamp, F; de Koning, I; Schudel, W J; Pepplinkhuizen, L. J Neural Transm, 1998, 105: 287-94].

Design and Progress of Trial

The current trial was designed with several improvements relative to its predecessor [Bentham, P W. International Clinical Psychopharmacology, 1990, 5: 261-72]: (1) A greater number of patients were enrolled, thus affording a larger data set for more accurate statistical analysis; (2) The cognitive tests employed, unlike those used in the earlier trial (which have since been shown to be unreliable), are widely accepted and routinely used in the fields of neurology and psychiatry (personal communication with J. Irwin, neuropsychologist); (3) The duration of the trial was six months rather than three months, increasing the likelihood of detecting a difference in the treatment group relative to the placebo group.

While Trp may benefit AD patients through a serotonergic pathway, as suggested in earlier clinical investigations [Bentham, P W. International Clinical Psychopharmacology, 1990, 5: 261-72., Porter, R J; Lunn, B S; Walker, L L; Gray, J M; Ballard, C G; O Brien, J T. American Journal of Psychiatry, 2000, 157: 638-40, Porter, R J; Lunn, B S; O'Brien, J T. Psychol Med, 2003, 33: 41-9, Fekkes, D; van der Cammen, T J; van Loon, C P; Verschoor, C; van Harskamp, F; de Koning, I; Schudel, W J; Pepplinkhuizen, L. J Neural Transm, 1998, 105: 287-94], it is also possible that Trp could bind to Aβ and inhibit fibrillogenesis. Being an “indole-anionic” compound, Trp could form one cation-π bond and one anionic-cationic interaction to the HHQK region of Aβ. This type of binding would involve a two-point pharmacophore of Trp, comprised of its indole and carboxylate groups, binding to two of the three basic residues of the HHQK region.

Twelve patients were enrolled in the current clinical trial. Patients were administered 1 g Trp (n=8) or placebo (n=4) twice a day for six months, and their cognitive abilities were assessed, using standardized tests, at baseline, three months and six months. All enrollment and testing was carried out at the Kingston General Hospital, Kingston, Ontario, Canada, under the supervision of the principal investigator, D. F. Weaver, M.D., Ph.D (the protocol is attached as Appendix A). The primary endpoint of the double-blind trial was to determine whether patients receiving Trp outperformed the controls, either by experiencing an improvement in cognition or a reduction in deterioration in cognitive abilities relative to the placebo group.

Methods

Participants

Eligible participants were those who met the clinical criteria of probable Alzheimer's disease as described in the Diagnostic and Statistical Manual of Mental Disorders, fourth edition (DSM-IV) [Anonymous, Diagnostic and Statistical Manual of Mental Disorders, 4th Edition (DSM-IV). 1994, Washington, D.C.: American Psychiatric Association] and in the National Institute of Neurologic and Communicative Disorders and Stroke and the Alzheimer's Disease Related Disorders Association (NINCDS-ADRDA) [McKhann, G; Drachman, D; Folstein, M; Katzman, R; Price, D; Stadlan, E M. Neurology, 1984, 34: 939-44]. Inclusion criteria also included: mild to moderate severity of dementia, as reflected by a Mini-Mental State Examination (MMSE) [Folstein, M F; Folstein, S E; McHugh, P R. J Psychiatr Res, 1975, 12: 189-98] score of 14 to 26; minimum one-year duration of symptoms; minimum age of 50 years; living at home or in an institution provided they had caregivers capable of attending each clinic visit and ensuring the administration of medication; able to perform the psychometric tests required; reasonably good nutritional status; vital signs (blood pressure and heart rate in sitting and standing positions), urinalysis, physical examination, and neurological evaluation must yield results within normal limits or determined as not clinically significant by the study physician for the patient's age and sex.

Patients were excluded from the trial if they had: any other cause of dementia, such as vascular dementia, as evidenced by Modified Hachinski Ischemia Scale [Rosen, W G; Terry, R D; Fuld, P A; Katzman, R; Peck, A. Ann Neurol, 1980, 7: 486-8] score greater than 4; depressive pseudementia and/or a history of more than one major depressive episode, according to DSM-IV; Creutzfeldt-Jakob disease; clinically important head injury within one year of onset of dementia; onset of dementia following cardiac arrest or heart surgery; Huntington's chorea or Parkinson's disease, evidenced by neurological examination; DSM-IV criteria for any major psychiatric disorder including schizophrenia, alcohol or substance abuse; history or current evidence of stroke; neurosyphilis or seropositivity for HIV; vitamin B₁₂ or folate deficiency; uncorrected hypothyroidism. Patients with clinically significant coexisting medical conditions including impaired renal, hepatic or gastrointestinal function were also excluded, as were patients with history or current evidence of sleep disorder.

The following medications were prohibited for safety reasons or to prevent a false positive result: investigational drugs for AD or for any other disorder, MAO inhibitors (e.g. phenelzine, tranylcypromine, noclobemide), SSRI antidepressants (e.g. fluoxetine, paroxetine), lithium, acetyl-cholinesterase inhibitors (e.g. donepezil, galantamine), ginkgo biloba, ginseng, vitamin E supplementation, estrogen for more than hormonal replacement, aspirin at doses greater than 650 mg/day, non-steroidal anti-inflammatory drugs (NSAIDS, e.g. ibuprofen, naproxen, diclofenac) for more than 30 consecutive days (allowed as required). Information on allowed concomitant treatments taken by patients was recorded at each visit.

Written informed consent was obtained from patients and their substitute decision-makers before the initiation of any testing procedures. The study was reviewed and approved by the Queen's University Health Sciences and Affiliated Teaching Hospitals Research Ethics Board, Kingston, Canada.

Study Design

This study was a 6-month, double-blind randomized trial in which eligible participants were randomly assigned to receive either Trp (1000 mg, twice daily) or an identical-appearing placebo. Recruiters were unaware of the assignment. The randomization ratio was two to one for Trp versus placebo, as has been used previously in AD clinical trials [Erkinjuntti, T; Kurz, A; Gauthier, S; Bullock, R; Lilienfeld, S; Damaraju, C V. Lancet, 2002, 359: 1283-90, Erkinjuntti, T; Kurz, A; Small, G W; Bullock, R; Lilienfeld, S; Damaraju, C V. Clin Ther, 2003, 25: 1765-82]. Doses consisted of a single capsule and were administered one hour before breakfast and at bed time each day. The medication was taken without food to prevent absorption competition from dietary amino acids such as phenylalanine [Kilberg, M; Haussinger, D, Mammalian Amino Acid Transport Mechanisms and Control. 1992, Plenum: New York. p. 166-7]. A dosage diary was filled out by the participant's caregiver to ensure compliance. After the initial screening, clinic visits took place at 0, 3 and 6 months. Blinding was maintained until all patients completed the trial.

The primary efficacy measures were the change in MMSE [Folstein, M F; Folstein, S E; McHugh, P R. J Psychiatr Res, 1975, 12: 189-98] and Alzheimer's Disease Assessment Scale, cognitive subpart (ADAS-Cog) [Rosen, W G; Mohs, R C; Davis, K L. Am J Psychiatry, 1984, 141: 1356-64]. The MMSE is a short standard assessment for diagnosing the presence and severity of cognitive impairment, and evaluates six domains of cognitive functioning: orientation, registration, attention, recall, language and constructional abilities. The maximum score is 30 points, with lower scores indicating a greater degree of cognitive impairment. The ADAS-Cog is an 11-item test battery that relies solely on the patient's ability to perform specific tasks during the administration of the test. It ranges from 0 to 70 points, with higher scores indicating a greater degree of cognitive impairment.

Secondary efficacy measures were the Alzheimer's Disease Cooperative Study—Clinical Global Impression of Change (ADCS-CGIC) [Schneider, L S; Olin, J T; Doody, R S; Clark, C M; Morris, J C; Reisberg, B; Schmitt, F A; Grundman, M; Thomas, R G; Ferris, S H. Alzheimer Dis Assoc Disord, 1997, 11 Suppl 2: S22-32], a rating designed to describe the change, as determined by the clinician, occurring in a patient from baseline; Neuropsychiatric Inventory (NPI) [Cummings, J L; Mega, M; Gray, K; Rosenberg-Thompson, S; Carusi, D A; Gombein, J. Neurology, 1994, 44: 2308-14], a test performed by a neuropsychologist to assess ten behavioural disturbances that can occur in patients with dementia: delusions, hallucinations, agitation/aggression, dysphoria, anxiety, elation/euphoria, apathy, disinhibition, irritability/lability and aberrant motor behaviour; Disability Assessment for Dementia (DAD) [Gelinas, I; Gauthier, L; McIntyre, M; Gauthier, S. Am J Occup Ther, 1999, 53: 471-81], a test used to quantitatively measure functional abilities in the activities of daily living, such as meal preparation and housework, in individuals with cognitive impairments; Functional Activities Questionnaire (FAQ) [Pfeffer, R I; Kurosaki, T T; Harrah, C H, Jr.; Chance, J M; Filos, S. J Gerontol, 1982, 37: 323-9], a caregiver-based measure of functional activities; and a clock-drawing test [Tuokko, H; Hadjistavropoulos, T; Miller, J A; Beattie, B L. J Am Geriatr Soc, 1992, 40: 579-84], an instrument that assesses a range of cognitive abilities, including executive functioning and visuospatial perception. At each visit after baseline, participants were also questioned about any adverse events experienced since beginning the trial.

Since Trp or its metabolites may have sedative [Wyatt, R J; Engelman, K; Kupfer, D J; Fram, D H; Sjoerdsma, A; Snyder, F. Lancet, 1970, 2: 842-6, Mannaioni, G; Carpenedo, R; Corradetti, R; Carla, V; Venturini, I; Baraldi, M; Zeneroli, M L; Moroni, F. Adv Exp Med Biol, 1999, 467: 155-67] or anti-depressant properties [Boman, B. Aust N Z J Psychiatry, 1988, 22: 83-97], the Pittsburgh Sleep Quality Index [Buysse, D J; Reynolds, C F, 3rd; Monk, T H; Berman, S R; Kupfer, D J. Psychiatry Res, 1989, 28: 193-213] and Cornell Depression Index [Alexopoulos, G S; Abrams, R C; Young, R C; Shamoian, C A. Biol Psychiatry, 1988, 23: 271-84] were performed to ensure that any change in a patient's dementia is unrelated to these potential effects.

Primary efficacy variables, the Pittsburgh Sleep Quality Index and Cornell Depression Index were measured at 0, 3 and 6 months, while secondary efficacy variables were measured at 0 and 6 months.

Statistical Analyses

All statistical comparisons were two-tailed, with p values <0.05 considered significant. Analysis was based on intention to treat, comparing mean change from baseline in primary and secondary efficacy measures between treatment and control groups by means of t-tests. All efficacy measures found significantly associated with treatment status were entered into an ANOVA model with baseline score and treatment status as independent variables and change in score as the dependent variable. Baseline characteristics were tested for significant differences between treatment and control groups by t-test (for continuous variables) and χ² for categorical variables. Within the treatment group, change in scores from baseline of those primary and secondary outcome efficacy measures found to be significantly associated with treatment status were tested for significance by means of two-tailed paired t-tests.

Results

The treatment and placebo groups had similar characteristics at baseline (Table 5). Although the treatment group consistently trended towards higher baseline scores on the cognitive tests, none of the differences reached significance.

TABLE 5
Baseline characteristics

| | Placebo | Trp | P |
|---|---|---|---|
| Characteristic | (n = 4) | (n = 8) | value |
| Demographics | | | |
| Women (%) | 2 (50) | 4 (50) | 1.0 |
| Age, mean (SD) | 72.8 (9.1) | 71.4 (8.3) | 0.80 |
| Weight, mean | 64.5 (5.8) | 70.0 (18.2) | 0.58 |
| (SD), kg | | | |

Cognitive function, mean score (SD)

| | | | |
|---|---|---|---|
| MMSE | 21.0 (3.4) | 22.4 (4.7) | 0.61 |
| ADAS-Cog | 22.8 (7.2) | 20.4 (8.7) | 0.65 |
| DAD | 67.8 (26.5) | 84.5 (22.2) | 0.27 |
| FAQ | 18.8 (6.7) | 10.9 (9.8) | 0.18 |
| NPI | 15.5 (8.7) | 17.8 (13.9) | 0.78 |
| Clock-drawing | 3.8 (4.2) | 6.2 (3.0) | 0.26 |

No adverse events were reported by patients taking either Trp or placebo, and no patients withdrew from the trial. All dosage diaries were returned indicating complete compliance. All patients entered the intention-to-treat analysis.

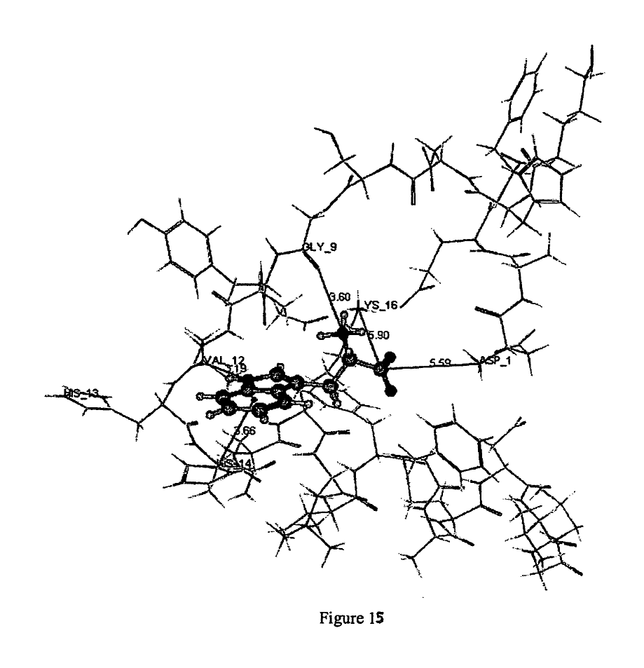

Patients assigned Trp improved in cognitive function relative to patients receiving placebo (Table 6, FIG. 15). In terms of the primary efficacy variables, the treatment group had a significantly improved change in MMSE score at 3 months (p=0.009) and 6 months (p<0.001), while the improvement in ADAS-Cog relative to controls was significant at 6 months (p=0.017), but not at 3 months (p=0.38). Improvements were also seen after 6 months on two of the five secondary efficacy measures: ADCS-CGIC scores were higher and change in clock test was improved in patients receiving Trp relative to the placebo group (Table 6). All significant improvements on primary and secondary efficacy measures remained significant when baseline test score was controlled for (data not shown).

TABLE 6
Efficacy outcomes after 3 and 6 months

| | | | | |
|---|---|---|---|---|
| | | | Treatment | |
| | | | difference | |
| Outcome | | | (Trp | |
| measure | Placebo (n = 4) | Trp (n = 8) | vs. placebo) | P value |

Mean (SE) change from baseline after 3 months

| | | | | |
|---|---|---|---|---|
| MMSE^{a} | −1.2 (0.5) | 0.8 (0.4) | 2.0 | 0.009 |
| ADAS-Cog^{b} | 0.5 (0.3) | −0.8 (0.9) | 1.3 | 0.39 |

Mean (SE) change from baseline after 6 months

| | | | | |
|---|---|---|---|---|
| MMSE^{a} | −3.2 (0.6) | 1.8 (0.5) | 5.0 | <0.001 |
| ADAS-Cog^{b} | 3.5 (1.0) | −2.8 (1.4) | −6.3 | 0.017 |
| DAD^{a} | −11.2 (9.0) | 1.5 (1.8) | 12.8 | 0.25 |
| FAQ^{b} | 5.2 (1.4) | 3.2 (2.0) | −2.0 | 0.53 |
| NPI^{b} | 22 (18) | 3 (3) | −19 | 0.38 |
| Clock-drawing^{a} | −1.8 (0.3) | 1.1 (0.4) | 2.9 | <0.001 |
| ADCS-CGIC^{c} | 6.5 (0.3) | 3.6 (0.3) | 2.9 | <0.001 |

^{a}Positive change indicates improvement.
^{b}Negative change indicates improvement.
^{c}Score at 6 months, not change from baseline (score is a measure of change). Lower score indicates improvement.

At 6 months, improvement in the treatment group from baseline was significant for MMSE (p=0.006) and clock test (p=0.015), though not for ADAS-Cog (p=0.097). The improvement in MMSE score at 3 months in people taking Trp likewise failed to reach significance (p=0.08).

No difference was found to exist in the moods of patients in the treatment versus the placebo group; no patients were depressed at baseline, as indicated by a Cornell Depression Index score of >5, while two patients taking Trp and one taking placebo were depressed after 6 months of treatment (i.e. 25% of each group). No sleep problems, identified by a score of >4 on the Pittsburgh Sleep Quality, were reported at baseline or at 6 months in either the treatment or placebo groups.

Discussion

This trial found that administration of Trp was of benefit to individuals with Alzheimer's disease. Cognitive abilities, measured by the MMSE and ADAS-Cog tests, improved in patients taking Trp, both relative to baseline and to patients receiving placebo.

The trial suffered from many limitations, particularly small size. While it was originally planned to enroll 30 patients in the trial (20 treatment, 10 placebo), limitations in the enrollment period only allowed for the recruitment of 12 patients. A larger cohort may have lead to smaller differences on baseline cognitive tests between the two groups; although none of the differences were found to be significant, there was a consistent trend towards greater cognitive abilities in the treatment group. Since people at more advanced stages of AD are known to decline faster [Mitnitski, A B; Graham, J E; Mogilner, A J; Rockwood, K. J Gerontol A Biol Sci Med Sci, 1999, 54: M65-9], this trend may account for some of the difference in cognitive outcomes between the two groups.

No difference was seen to exist between the placebo and treatment groups in terms of the number of people reporting sleep disturbances or depression, either at baseline or at completion of the trial. Since both are binary measures, however, moderate improvements or declines in sleep quality or mood could not be detected. If such discrepancies existed between the treatment and control groups, they could have had an effect on the measured efficacy of Trp.

In the earlier trial of Trp in patients with AD [Bentham, P W. International Clinical Psychopharmacology, 1990, 5: 261-72], no difference in depression, measured by the Montgomery and Asberg Depression Rating Scale [Montgomery, S A; Asberg, M. Br J Psychiatry, 1979, 134: 382-9], was found between treatment and placebo groups. Data on sleep quality were incomplete and therefore not analyzed.

Not only did all the primary and secondary efficacy tests trend towards, or were found significantly associated with, the treatment group outperforming the placebo group, but five of the seven tests (all but FAQ, NPI) suggested an increase in cognitive abilities over the course of the trial for those receiving Trp. Two of these efficacy measures (MMSE and clock test) were found to be significantly improved at 6 months relative to baseline.

The efficacy measures in which Trp showed a significant benefit (MMSE, ADAS-Cog, clock test and CGIC) all evaluate higher-level cognitive function, while the tests that showed no benefit from Trp (FAQ, DAD and NPI) evaluate behavioural disturbances and functional abilities. While it may be that Trp acts to improve cognitive but not non-cognitive faculties, other explanations may account for this finding. Measuring behavioural disturbances, for instance, is inherently associated with greater uncertainty than evaluating specific cognitive skills such as arithmetic or word recall. The greater uncertainty in measuring non-cognitive functioning may have reduced the ability to detect differences between the treatment and placebo groups in the FAQ, DAD and NPI tests, especially in a study with such low power.

This is not the first account of Trp benefiting patients with dementia. In the only prior randomized clinical trial in patients with AD [Bentham, P W. International Clinical Psychopharmacology, 1990, 5: 261-72], the author found a significant correlation between increase in Trp plasma levels and increase in scores on the Information, Memory, Concentration test [Blessed, G; Tomlinson, B E; Roth, M. Br J Psychiatry, 1968, 114: 797-811]. Several other efficacy measures used in the trial trended towards an improvement in the treatment group relative to the placebo group. In an earlier uncontrolled study, Shaw et al. found an “unequivocal improvement in blind global assessment” in six of 29 patients with advanced senile dementia, though not specifically AD, after daily Trp administration (24 mg/kg) [Shaw, D M; Tidmarsh, S F; Karajgi, B M; Sweeney, E A; Williams, S; Elameer, M; Twining, C. British Journal of Psychiatry, 1981, 139: 580-2]. None of the patients taking placebo showed improvement.

Conversely, a double-blind crossover trial of Trp in patients with dementia showed the drug to be of no benefit [Smith, D F; Stromgren, E; Petersen, H N; Williams, D G; Sheldon, W. Acta Psychiatr Scand, 1984, 70: 470-7]. It is possible that differences in trial design account for the lack of effect: patients were all residing in a nursing home; the duration of the trial was only one month; patients with any form of dementia, rather than specifically AD, were enrolled in the study, including some whose dementia was very advanced; dosing was only performed once daily; Trp was co-administered with a high-protein food (yogurt or chocolate milk); and many patients suffered from depression. Of particular interest, however, the efficacy measures used were nurse [Plutchik, R; Conte, H; Lieberman, M; Bakur, M; Grossman, J; Lehrman, N. J Am Geriatr Soc, 1970, 18: 491-500] and psychologist [Gotestam, K G. Acta Psychiatr Scand Suppl, 1981, 294: 54-63] ratings of patients' behaviour and functional abilities. As mentioned above, Trp was found in this trial to be ineffective at improving these domains, as reflected by scores on the FAQ, DAD and NPI tests.

The mechanism of action of Trp in AD has been discussed. Authors of earlier trials [Bentham, P W. International Clinical Psychopharmacology, 1990, 5: 261-72, Porter, R J; Lunn, B S; Walker, L L; Gray, J M; Ballard, C G; O Brien, J T. American Journal of Psychiatry, 2000, 157: 638-40, Porter, R J; Lunn, B S; O'Brien, J T. Psychol Med, 2003, 33: 41-9, Fekkes, D; van der Cammen, T J; van Loon, C P; Verschoor, C; van Harskamp, F; de Koning, I; Schudel, W J; Pepplinkhuizen, L. J Neural Transm, 1998, 105: 287-94] proposed the drug could act through a serotonergic pathway, i.e. increased Trp intake would lead to greater serotonin synthesis. However, while clinical trials of selective serotonin reuptake inhibitors (SSRIs) in AD [Lyketsos, C G; DelCampo, L; Steinberg, M; Miles, Q; Steele, C D; Munro, C; Baker, A S; Sheppard, J M; Frangakis, C; Brandt, J; Rabins, P V. Arch Gen Psychiatry, 2003, 60: 737-46, Teri, L; Logsdon, R G; Peskind, E; Raskind, M; Weiner, M F; Tractenberg, R E; Foster, N L; Schneider, L S; Sano, M; Whitehouse, P; Tariot, P; Mellow, A M; Auchus, A P; Grundman, M; Thomas, R G; Schafer, K; Thal, L J. Neurology, 2000, 55: 1271-8, Nyth, A L; Gottfries, C G. Br J Psychiatry, 1990, 157: 894-901, Olafsson, K; Jorgensen, S; Jensen, H V; Bille, A; Arup, P; Andersen, J. Acta Psychiatr Scand, 1992, 85: 453-6] have shown improvement in a number of noncognitive symptoms, they have shown no effect on cognition. Furthermore, neither this trial, nor its predecessor [Bentham, P W. International Clinical Psychopharmacology, 1990, 5: 261-72] found a difference in depression between the treatment and placebo groups. These findings suggest a non-serotonergic mechanism of action for Trp's activity in AD.

As a non-toxic, readily-absorbed and naturally abundant compound, it is possible that Trp has the potential to be a future treatment for AD.

### EXAMPLE 17

Binding energies of L-Trp to the HHQK region of Aβ were calculated using the CHARM27 force field and explicit solvation (Table 7). Using CHARMM22, an earlier version of CHARMM, the binding energies of L-Trp to PDB structures of Aβ were similar to those for sodium 1,3-propanedisulfonate, a known Aβ anti-aggregant (Kisilevsky et al., Nature Medicine, 1:143-148, 1995).

TABLE 7
Binding energies of L-Trp to HHQK region of Aβ using the
CHARMM27 force field and explicit solvation.

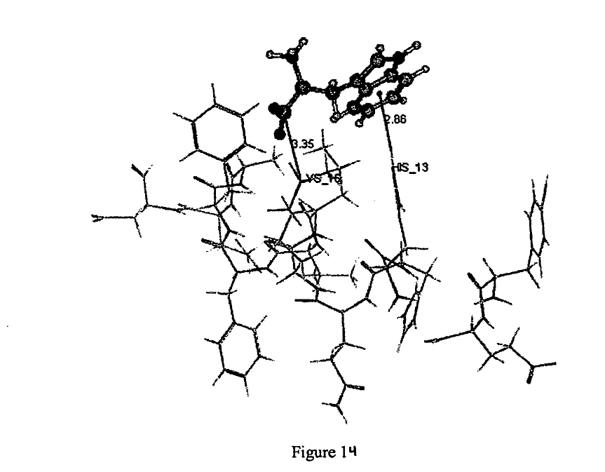

L-Trp Binding at:

| PDB Structure | His13-His14 | His13-Lys16 |
|---|---|---|
| 1AMB | −54.4 | −43.5 |
| 1AMC | −44.6 | −34.3 |
| 1AML | −35.6 | −22.3 |
| 1BA4 | −27.1 | −46 |
| 1IYT | −36.8 | −17.3 |
| 2BP4 | −32.7 | −48.6 |
| Notes: | | |
| Energies are in kcal/mol. | | |
| CHARMM27 force field is a molecular mechanics computer program. | | |
| Ref.: A. D. MacKerell et al., Journal of Physical Chemistry B, 102: 3586-3616, 1998. Explicit solvation means water molecules were present during the binding of L-Trp to Aβ. | | |
| PDB = Brookhaven Protein Databank [HM Berman et al. (2000). Nucleic Acid Research 28: 235-42]. | | |
| These 6 different PDB files are of various lengths of Aβ, and were generated from NMR experiments under different conditions (pH, solvent, temp). Two different binding motifs of L-Trp to the HHQK region of Aβ are shown in FIGS. 14 and 15. FIG. 14 gives the “typical” interaction found for binding at His₁₃ and Lys₁₆ of Aβ, here to PDB structure 1AML. Significant binding interactions at His₁₃ and His₁₄ were also found. | | |
| # FIG. 15 is an alternative interaction found at residues His₁₄ and Lys₁₆ of the PDB structure 1BA4. Asp₁, Gly₉ and Val₁₂ also participate in binding. | | |

### EXAMPLE 18

### Molecular Minimization Results

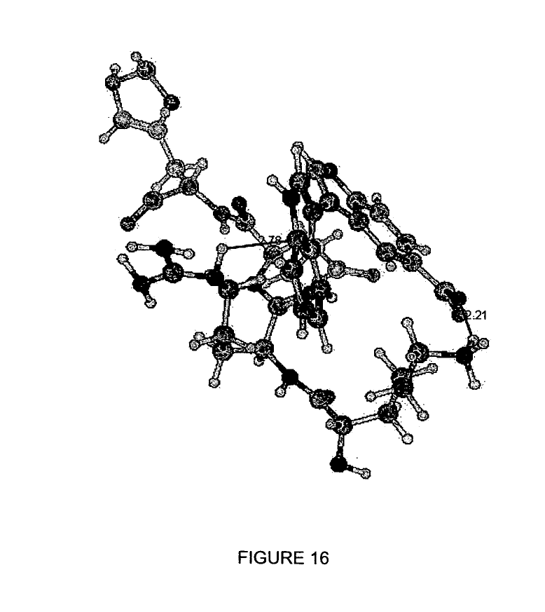

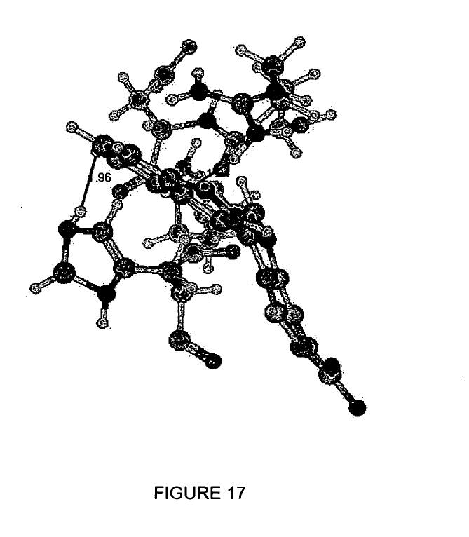

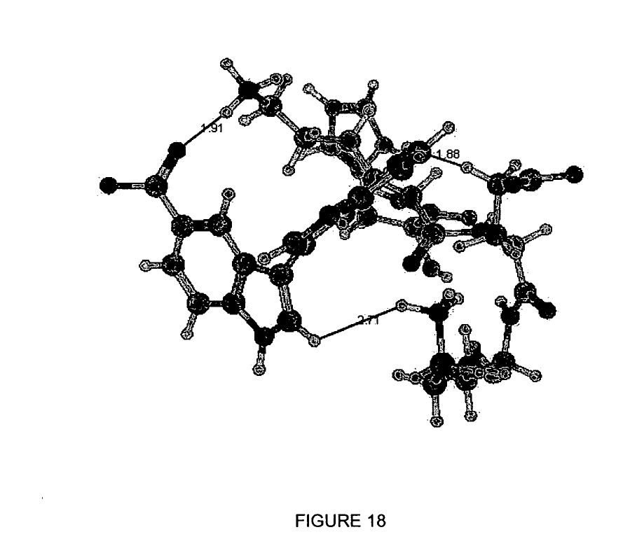

It has been suggested that Alzheimer's Disease (AD) is a multifactorial condition, with a diverse group of proteins implicated in its pathogenesis. Stephenson et. al. (2005. FEBS Lett 579:1338-42) have identified a common BBXB motif present in the peptide sequences of 27 such AD associated proteins. It is from this group of BBXB-containing proteins (BCPs) that several were selected for a series of in silico calculations with the objective of determining the ability of a novel molecule, 0c, to bind to their BBXB receptors and potentially neutralize their action and toxicity. FIG. 16 depicts the interaction of 0c with KREH receptor of B7-1. FIG. 17 depicts the interaction of 0c with RDHH receptor of ICAM-1. FIG. 18 depicts interaction of 0c with HKEK receptor of IL-1R1.

Methods

A collection of molecular systems were constructed, each consisting of a three dimensional structure of a BCP, obtained from the ExPASy Protein Knowledgebase (Apweiler R et al. 2004. Nucleic Acids Research 32:115-9), with a manually modelled molecule of 0c placed in close proximity to a BBXB receptor of said protein. Subsequently, these systems were each minimized using the Chemical Computing Group Molecular Operating Environment (MOE) software (The Chemical Computing Group, Montreal, Canada, 2000), and the resulting bonding energy and separation distance of 0c to BBXB receptor interaction calculated. These values were then compared to separation distance and bonding energy values calculated for a control system consisting of an interaction between the HQHK receptor of IL-1R1 and pentane, a molecule not expected to have strong interactions at BBXB receptors. Representative interactions between 0c and some BCPs are found in FIGS. 16, 17 and 18.

TABLE 8
Results of in silico simulations between 0c and a series of BBXB receptors
found in various proteins believed to be associated in the pathogenesis of
Alzheimer's Disease.

| | | | | |
|---|---|---|---|---|
| | | | Binding | Approximate |
| | | | Energy | Separation |
| Protein | PDB File | Target Receptor | (kcal/mol) | (Å) |

Substrate: 0C

| | | | | |
|---|---|---|---|---|
| B7-1 | 1DR9 | KREH^{b} | −30.6 | 2 |
| BHMT | 1LT7 | RARK^{b} | −5.7 | 8 |
| | | RARK^{ab} | −17.4 | 3 |
| C1qA | 1PK6 | KKGH | −12.3 | 2 |
| HFE | 1A6Z | HKIR | −1.0 | 6 |
| ICAM-1 | 1IAM | RDHH^{b} | −30.5 | 3 |
| | | RRDH | −12.9 | 3 |
| IFN-g | 1EKU | KKKR^{a} | −8.8 | 2 |
| | | KKKR^{ab} | −24.3 | 3 |
| IL-12B | 1F42 | KSKR | −0.4 | 6 |
| | | HKLK^{b} | −15.9 | 2 |
| IL-13 | 1GA3 | HLKK^{b} | −18.6 | 3 |
| IL-4 | 1BBN | HHEK^{b} | −15.5 | 3 |
| IL-1R1 | 1ITB | HKEK^{b} | −43.8 | 3 |
| | | HQHK^{b} | −17.0 | 2 |
| MIP-1α | 1B53 | KRSR^{b} | −30.7 | 3 |
| MIP-1β | 1HUM | KRSK^{a} | −6.7 | 2 |
| | | KRSK^{ab} | −16.1 | 2 |
| Neprilysin | 1D19 | HCRK^{b} | −28.8 | 3 |
| | | KKCR^{b} | −44.5 | 3 |
| | | KKLR^{b} | −35.9 | 3 |
| S100β | 1UWO | HKLK^{b} | −34.8 | 3 |
| | | KLKK | −13.3 | 8 |
| SDF-1 | 2SDF | KHLK | −10.5 | 3 |
| Transferrin | 1N84 | RGKK^{b} | −18.7 | 3 |
| A₁-ACT | 1AS4 | KRWR^{b} | −25.8 | 3 |

Control Substrate: pentane

| | | | | |
|---|---|---|---|---|
| IL-1R1 | 1ITB | HKEK | −9.8 | 3 |

^{a}Multiple occurrences of sequence in PDB file studied
^{b}Denotes favourable interaction with 0c, defined as <−15 kcal/mol

TABLE 9
Parameters passed to MOE for purposes of molecular
minimization of sample systems.
Initial Refinement Parameters

| | | | |
|---|---|---|---|
| Force Field | CHARMM27 | | |
| Gradient | 0.05 | | |
| Partial Charge Calculation | Enabled | | |
| Atom Tethering | Enabled | Backbone | |
| Bonded | Enabled | | |
| van der Waals | Enabled | | |
| Electrostatics | Enabled | | |
| Restraints | Enabled | | |
| Cutoff | Enabled | On: 8 | Off: 10 |
| Solvation | Distance | Dielectric: 1 | Exterior: 80 |
| Scale | Like: 1 | Unlike: 0 | Wild: 1 |

Final Refinement Parameters

| | | | |
|---|---|---|---|
| Force Field | CHARMM27 | | |
| Gradient | 1 | | |
| Partial Charge Calculation | Enabled | | |
| Atom Tethering | N/A | | |
| Bonded | Enabled | | |
| van der Waals | Enabled | | |
| Electrostatics | Enabled | | |
| Restraints | Enabled | | |
| Cutoff | Enabled | On: 8 | Off: 10 |
| Solvation | Gas Phase | Dielectric: 1 | Exterior: 80 |
| Scale | Like: 1 | Unlike: 0 | Wild: 1 |
| Water Soak | Solute: All | Mode: Box | Width: 5 |

Minimizations were carried out using the CHARMM27 force field (Brooks B R et al. 1983. J Comput Chem 4:187-217; MacKerell A D Jr. 1998. CHARMM: The Energy Function and Its Parameterization with an Overview of the Program. In Encyclopedia of Computational Chemistry, Schleyer, P R et al., eds., John Wiley & Sons, Chichester, Vol. 1, pp 271-277), and consisted of a two-step process (Table 9). Initially, an optimization was conducted on the system without solvent molecules, so that the binding geometries could be refined, producing a substrate that was oriented in a chemically realistic fashion. The refined system was then explicitly solvated with water molecules in order to simulate physiological circumstances, and again optimized to further improve system geometries. The resulting geometries, which better represent molecular behaviour in vivo, were then analysed, and binding energies and separation distances were calculated (Table 9). A favourable interaction was defined as one in which the binding energy of 0c to the BBXB receptor was greater than 15 kcal/mol (i.e. the system energy was <−15 kcal/mol), with a substrate to receptor separation distance of less than or equal to 3 Å. Using these criteria, favorable interactions were found to exist between 0c and 17 of the 26 BCP systems analyzed. The pentane-to-HQHK control interaction was only found to have a binding energy of −9.8 kcal/mol.

The in silico simulations performed suggest that 0c is able to bind to the BBXB motifs of a number of proteins involved in AD pathogenesis, and that the compound and ones with similar structures may therefore be beneficial in treating AD.

### EXAMPLE 20

### Molecular Modeling Study: Aromatic Groups for Cation-π Binding to HHQK

A study was carried out that examined, via ab intio methods, the interaction of methylammonium (MA), the end of lysine's sidechain, and 4-methylimidazolium (4-MI), the protonated sidechain of histidine, with a series of 11 monocylic and bicyclic aromatic species (benzene, pyridine, pyrrole, thiophene, furan, naphthalene, indole, quinoline, isoquinoline, benzothiophene and benzofuran). The goal of the study was to evaluate, using Gaussian98 (Revision A.9. 1998, Gaussian Inc., Pittsburgh, Pa., U.S.A.), the ability of different aromatic systems to bind to one of the histidine or lysine residues in the HHQK region of Aβ.

Methods

MA and 4-MI and each aromatic group studied were individually geometry-optimized at the restricted Hartree-Fock (RHF) level using the 3-21G basis set in the Gaussian98 computer program. Once optimized, the single-point RHF and MP2 energies of the binding complexes and those of the cations and the aromatic species on their own were calculated using the 6-31G(d) basis set. Binding energies were determined by subtracting the sum of the monomer energies from the energy of each complex.

Results

The trend in binding strength at the MP2/6-31G(d) level, using the most energetically favourable interaction for each aromatic, be it charge-dipole or cation-π in nature, is shown in Table 10. Pyridine and pyridine-containing aromatic groups (quinoline and isoquinoline) formed the strongest interactions, though these were charge-dipole in nature, and would therefore be expected to be of much smaller magnitude in an aqueous environment (i.e. in vivo). Indole formed the strongest cation-π interactions, followed by naphthalene and pyrrole. The magnitude of these may be equal to or greater than the charge-dipole interactions formed by pyridine, quinoline and isoquinoline when solvation is taken into account.

Accordingly, indole was a preferred aromatic group incorporated into certain compounds employed in methods of the invention.

TABLE 10
Binding of MA and 4-MI to aromatic systems. MP2/6-31G(d)//RHF/3-21G
binding energies of MA and 4-MI to the aromatic systems studied. Only
the most energetically favourable binding complex is included.

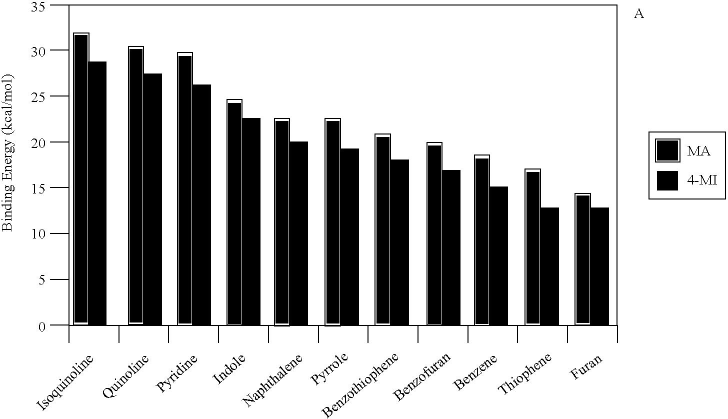

Many other variations of the present invention will be apparent to those skilled in the art and are meant to be within the scope of the claims appended hereto.

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

1. A method for treating a protein folding disorder comprising administering a compound of formula (I) to a subject: 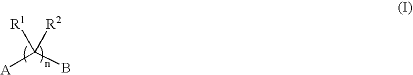
wherein A and B are independently a mono- or bicyclic aromatic or heteroaromatic substituent; wherein n=0 or 1; wherein, when n=1, R¹ and R² are independently hydrogen, alkyl, cycloalkyl, alkoxy, hydroxy, halogen, or aryl; wherein A is substituted by A¹₍ₓ₎ and B is substituted by B¹_{(y)}; wherein x and y are independently an integer from 0 to 4; and
A¹₍ₓ₎ and B¹_{(y)} are each independently, for each value of x and y, selected from the group consisting of alkyl, alkenyl, alkynyl, cycloalkyl, aryl, alkylcarbonyl, alkoxy, trihalomethoxy, aryloxy, arylcarbonyl; alkoxycarbonyl, aryloxycarbonyl, amino, hydroxy, thio, thioether, cyano, nitro, halogen, carboxylic acid and sulfonic acid;
or a pharmaceutically acceptable salt thereof;
wherein the subject is treated for the protein folding disorder.

2. The method of claim 1, wherein A and B are independently selected from the group consisting of phenyl, pyridyl, pyrrolyl, thiophenyl, furanyl, triazolyl, indolyl, naphthyl, benzofuranyl, quinolinyl and isoquinolinyl.

3. The method of claim 2, wherein at least one of A and B are indolyl.

4. The method of claim 3, wherein both A and B are indolyl.

5. The method of claim 4, wherein the compound of formula (I) is:

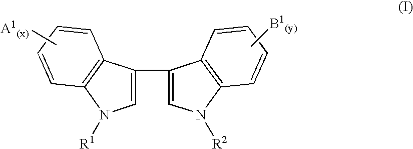

6. The method of claim 5, wherein x is 1 and A¹ is at the 5, 6 or 7 position.

7. The method of claim 6, wherein x is 1 and A¹ is CO₂H.

8. The method of claim 5, wherein B¹ is at the 5 or 6 position.

9. The method of claim 8, wherein B is selected from the group consisting of halogen, alkyl, alkoxy, aryl, thio, thioether, and trihalomethoxy.

10. The method of claim 4, wherein the compound of formula (I) is:

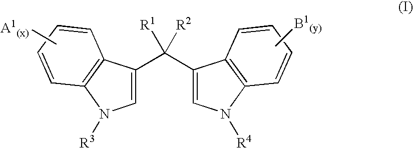

11. The method of claim 10, wherein x is 1 and A¹ is at the 5 position;
wherein R¹ and R² are independently hydrogen, alkyl, cycloalkyl, alkoxy, hydroxy, halogen, or aryl.

12. The method of claim 11, wherein x is 1 and A¹ is CO₂H.

13. The method of claim 10, wherein B¹ is at the 5 or 6 position.

14. The method of claim 13, wherein B¹ is selected from the group consisting of halogen, alkyl, alkoxy, aryl, thio, thioether, and trihalomethoxy.

15. The method of claim 4, wherein the compound of formula (I) is:

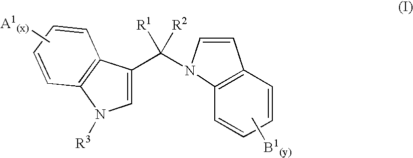

16. The method of claim 15, wherein x is 1 and A¹ is at the 5 position;
wherein R¹ and R² are independently hydrogen, alkyl, cycloalkyl, alkoxy, hydroxy, halogen, or aryl.

17. The method of claim 16, wherein x is 1 and A¹ is CO₂H.

18. The method of claim 15, wherein B¹ is at the 5 or 6 position.

19. The method of claim 18, wherein B¹ is selected from the group consisting of halogen, alkyl, alkoxy, aryl, thio, thioether, and trihalomethoxy.

20. The method of claim 15, wherein y is 1 and B¹ is at the 5 position;
wherein R¹ and R² are independently hydrogen, alkyl, cycloalkyl, alkoxy, hydroxy, halogen, or aryl.

21. The method of claim 20, wherein y is 1 and B¹ is CO₂H.

22. The method of claim 21, wherein A¹ is at the 5 or 6 position.

23. The method of claim 22, wherein A¹ is selected from the group consisting of halogen, alkyl, alkoxy, aryl, thio, thioether, and trihalomethoxy.

24. The method of claim 1, wherein the compound of formula I is selected from the group consisting of:
3,3′-bis-indolyl;
5-methoxy-3-(5-methoxy-indol-3-yl)-indole;
3-(5-bromo-indol-3-yl)-indole-5-carboxylic acid;
3-(indol-3-yl)-indole-5-carboxylic acid;
3-(5-methoxy-indol-3-yl)-indole-5-carboxylic acid;
3-(5-fluoro-indol-3-yl)-indole-5-carboxylic acid;
3-(5-chloro-indol-3-yl)-indole-5-carboxylic acid;
3-(5-methyl-indol-3-yl)-indole-5-carboxylic acid;
3-(5-(trifluoromethoxy)-indol-3-yl)-indole-5-carboxylic acid;
3-(indol-3-yl)-indole-6-carboxylic acid; and
a pharmaceutically acceptable salt thereof.

25. The method of claim 1, wherein the compound of formula I is selected from the group consisting of:
di-(indol-3-yl)methane;
3-((indol-1-yl)methyl)-indole;
bis(5-methoxy-indol-3-yl)methane;
5-methoxy-3-((5-methoxy-indol-1-yl)methyl)-indole;
3-((indol-3-yl)methyl)indole-5-carboxylic acid;
bis-(indole-5-carboxylic acid-3-yl)methane;
bis-(5-hydroxy-indol-3-yl)methane;
1,2-di-(indol-3-yl)ethane;
3-(2-(5-bromo-indol-3-yl)ethyl)-indole-5-carboxylic acid;
1,2-bis-(indole-5-carboxylic acid-3-yl)ethane; and
a pharmaceutically acceptable salt thereof.

26. The method of claim 1, wherein the protein folding disorder being treated is a neurodegenerative disease.

27. The method of claim 26, wherein the neurodegenerative disease is selected from the group consisting of Alzheimer's disease, dementia, Parkinson's disease, Huntington's disease, prion-based spongiform encephalopathy and a combination thereof.

28. The method of claim 26, wherein the neurodegenerative disease is Alzheimer's disease.

29-87. (canceled)
